(19) United States
(12) Patent Application Publication
(10) Pub. No.: US 2023/0414598 A1
(43) Pub. Date: Dec. 28, 2023

(54) COMBINATION THERAPIES WITH CBL-B INHIBITOR COMPOUNDS AND ANTIEMETIC AGENTS

(71) Applicant: NURIX THERAPEUTICS, INC., San Francisco, CA (US)

(72) Inventor: Robert BROWN, San Francisco, CA (US)

(21) Appl. No.: 18/213,177

(22) Filed: Jun. 22, 2023

Related U.S. Application Data
(60) Provisional application No. 63/354,517, filed on Jun. 22, 2022.

(51) Int. Cl. A61K 31/454; A61K 31/4178; A61K 31/439; A61P 1/08; A61K 31/473

(52) U.S. Cl. CPC A61K 31/454; A61K 31/4178; A61K 31/439; A61P 1/08; A61K 31/473

(57) ABSTRACT

The present disclosure relates to combination therapies with Cbl inhibitor compounds, and compositions and kits comprising combinations with the Cbl compounds. Also provided are methods of using the combinations with Cbl compounds and compositions thereof, such as in therapeutic methods. Therapies include Cbl inhibitor compounds in combination with a serotonin receptor antagonist.

### CROSS-REFERENCE TO RELATED APPLICATIONS

This application claims the benefit of U.S. provisional application no. 63/354,517, filed Jun. 22, 2022, the contents of which is hereby incorporated by reference in its entirety.

### FIELD

Provided herein are combination therapies with Cbl inhibitor compounds, compositions for administering the same, including pharmaceutical compositions, and kits for administering the same. The methods and compositions are useful for the treatment and prevention of cell proliferation and cancer.

### BACKGROUND

Casitas B-lineage lymphoma proto-oncogene-b (Cbl-b) is an E3 ubiquitin ligase that negatively regulates T-cell activation (Wallner et al., Clin. Dev. Immunol., 2012: 692639). Certain Cbl-b inhibitor compounds have shown promise for several potential immunotherapy applications through enhancing T-cell mediated anti-tumor activity by lowering the activation threshold of T-cells in a suppressive tumor microenvironment where Cbl-b plays a role in the downregulation of T-cells. Because of the mechanism of Cbl-b inhibitors, they have the potential to enhance, and in certain cases, to synergize the efficacy of another cancer therapeutic. Cancer therapeutics are known to cause nausea, gastrointestinal distress, among other adverse side effects. Accordingly, there is a need for a treatment therapy that is an effective cancer therapeutic while simultaneously treating the negative side effects of such therapeutic.

### SUMMARY

Provided herein are Cbl inhibitor compounds for use in combination with a serotonin receptor antagonist to treat cancer. As demonstrated in the Examples herein, in certain embodiments, a combination of a Cbl inhibitor compound with a serotonin receptor antagonist substantially improves the patient experience of therapeutically effective doses of a Cbl inhibitor. Also provided herein are methods for treating a patient with a combination of a Cbl inhibitor and a serotonin receptor antagonist. The methods can optionally include tapering off the amount of the serotonin receptor antagonist, such that subsequent doses of the Cbl inhibitor do not need the accompaniment of the serotonin receptor antagonist. In other words, the initial dose or doses of the serotonin receptor antagonist may continue to mitigate the adverse effects even when subsequent doses of Cbl inhibitor are administered with reduced doses of the serotonin receptor antagonist or even without the serotonin receptor antagonist. The methods can also optionally include dose escalation of the Cbl inhibitor with or without administration of the serotonin receptor antagonist and/or tapering of the serotonin receptor antagonist which may also mitigate the adverse effects.

While not intending to be bound by any particular theory of operation, unlike many other chemotherapeutics, Cbl inhibitor compounds described herein do not produce nausea directly by irritating the digestive track. Instead, the Cbl inhibitor compounds activate the serotonin receptor in the gut. Rather than the Cbl inhibitor compounds directly causing the nausea, the over activation of the serotonin receptor may be the actual cause of the increase in nausea and other adverse digestive symptoms (e.g., diarrhea, vomiting, etc.). In certain embodiments described herein, the administration of a serotonin receptor antagonist during at least a portion of the duration of the administration of the Cbl inhibitor compound has been discovered to train the gut to better tolerate the Cbl inhibitor compound.

Further, the co-administration of the serotonin receptor antagonist with the Cbl inhibitor compound has a synergistic effect. Specifically, the co-administration of the serotonin receptor antagonist has the effect of increasing the exposure of the Cbl inhibitor to the patient. In certain embodiments, a patient effectively receives a higher loading dose without administration of higher doses of the Cbl inhibitor compound. In certain embodiments, when the serotonin receptor antagonist is gradually decreased while the Cbl inhibitor dose is maintained. In certain embodiments, the effect of the Cbl inhibitor can be controlled by the dose of the serotonin receptor antagonist, for instance without changing the actual dose of the Cbl inhibitor compound. In certain embodiments, the Cbl inhibitor dose is increased in steps to modulate the effect of the Cbl inhibitor. In certain embodiments, the dose escalation is in combination with the serotonin receptor antagonist. In certain embodiments, the dose escalation is not in combination with the serotonin receptor antagonist.

In certain embodiments, the duration of the administration of the Cbl inhibitor compound is greater than a duration of the administration of the serotonin receptor antagonist. In an example, the duration of the administration of the serotonin receptor antagonist is periodically reduced during at least a portion of the duration of the administration of the Cbl inhibitor compound.

In one aspect, provided herein are methods of using the Cbl inhibitor compounds in combination with a serotonin receptor antagonist to treat cancer. In certain embodiments, the serotonin receptor antagonist is a serotonin inhibitor.

In another aspect, provided are kits or compositions comprising a Cbl inhibitor compound and a serotonin receptor antagonist. In certain embodiments, the Cbl compound and the serotonin receptor antagonist are in separate pharmaceutical compositions. In certain embodiments, the Cbl compound and the serotonin receptor antagonist are administered separately. In certain embodiments, the Cbl compound and the serotonin receptor antagonist are administered cyclically. In certain embodiments, the compositions are pharmaceutical compositions. Any suitable pharmaceutical composition may be used. In certain embodiments, the pharmaceutical composition for the Cbl compound is a composition for oral administration. In a particular embodiment, the serotonin receptor antagonist is an agent that prevents nausea. In a particular embodiment, the serotonin receptor antagonist is a serotonin inhibitor.

The methods, kits, and compositions are useful for inhibiting cell proliferation while mediating nausea. In certain embodiments, the methods, kits, and compositions are useful for treating cancer.

### BRIEF DESCRIPTION OF THE DRAWINGS

FIG. 1 depicts pharmacokinetic data for Compound 23. LLOQ=0.05 ng/mL.

### DETAILED DESCRIPTION OF THE EMBODIMENTS

### 1. Definitions

Unless otherwise defined, all terms of art, notations, and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

It is understood that aspects and embodiments described herein as “comprising” include “consisting of” and “consisting essentially of” embodiments.

As used herein and in the appended claims, the singular forms “a,” “an,” and “the” include plural referents unless otherwise indicated or clear from context. For example, “an” excipient includes one or more excipients.

Reference to “about” a value, encompasses from 90% to 110% of that value. For instance, about 50 billion cells refers to 45 to 55 billion cells, and includes 50 billion cells. For instance, a temperature of “about 100 degrees” refers to a temperature of about 90 degrees to about 110 degrees.

When numerical ranges of compounds are given, all compounds within those numerical limits designated “a” and “b” are included, unless expressly excluded. For example, reference to compounds 9-13 refers to compounds 9, 10, 11, 12, and 13.

The term “antiemetic” refers to a compound that reduces nausea, vomiting, and other gastrointestinal distress. Illustrative examples of antiemetics include antagonists of serotonin, dopamine, histamine, muscarinic and neurokinin systems, corticosteroids, and benzodiazepines.

The term “binding antagonist” in reference to a target refers to a molecule that decreases, blocks, inhibits, abrogates or interferes with signal transduction resulting from the interaction of such molecule with the target or with one or more the target's binding partners.

The term “CBL” refers to any Casitas B-lineage lymphoma gene encoding one of the CBL family of proteins. CBL family proteins include c-Cbl and Cbl-b.

The term “Cbl-b” as used herein refers to a Cbl-b protein. The term also includes naturally occurring variants of Cbl-b, including splice variants or allelic variants. The term also includes non-naturally occurring variants of Cbl-b, such as a recombinant Cbl-b protein or truncated variants thereof, which generally preserve the binding ability of naturally occurring Cbl-b or naturally occurring variants of Cbl-b (e.g., the ability to bind to an E2 enzyme). Sequences include NM_170662, NM_001321786, NM_001321788, NM_001321789, NM_001321790, and NM_001033238 (mRNA); and NP_001308715, NP_001308717, NP_001308718, NP_001308719, and NP_001308720 (protein).

The term “c-Cbl” as used herein refers to a c-Cbl protein. The term also includes naturally occurring variants of c-Cbl, including splice variants or allelic variants. The term also includes non-naturally occurring variants of c-Cbl, such as a recombinant c-Cbl protein or truncated variants thereof, which generally preserve the binding ability of naturally occurring c-Cbl or naturally occurring variants of c-Cbl (e.g., the ability to bind to an E2 enzyme). Sequences include NM_005188 human and NM_007619 mouse(mRNA); and NP_005179 human and NP_013645 mouse (protein).

“Cbl inhibitor” as used herein refers to a molecule that inhibits the activity of Cbl-b, c-Cbl, or both Cbl-b and c-Cbl proteins.

“Enhancing T-cell function” means to induce, cause, or stimulate a T-cell to have a sustained or amplified biological function, or renew or reactivate exhausted or inactive T-cells. Examples of enhanced T-cell function include increased T-cell activation (e.g., increased cytokine production, increased expression of T-cell activation markers, etc.), increased T-cell proliferation, decreased T-cell exhaustion, and/or decreased T-cell tolerance relative to the state of the T-cells before treatment with a Cbl-b inhibitor compound. Methods of measuring enhancement of T-cell function are known in the art.

As used herein, the term “inhibits growth” (e.g. referring to cells, such as tumor cells) is intended to include any measurable decrease in cell growth (e.g., tumor cell growth) when contacted with a compound or combination described herein, as compared to the growth of the same cells not in contact with the same compound or combination. In certain embodiments, growth may be inhibited by at least about 10%, 20%, 30%, 40%, 50%, 60%, 70%, 80%, 90%, 99%, or 100%. The decrease in cell growth can occur by a variety of mechanisms, including but not limited to apoptosis, necrosis, and/or effector function-mediated activity.

“Proliferation” is used herein to refer to the proliferation of a cell. Increased proliferation encompasses the production of a greater number of cells relative to a baseline value. Decreased proliferation encompasses the production of a reduced number of cells relative to a baseline value. In certain embodiments, the cell is an immune cell such as a T-cell and increased proliferation is desired. In certain embodiments, the cell is a cancer cell and reduced proliferation is desired.

“Serotonin receptor antagonist” is used herein to refer to a serotonin inhibitor. In certain embodiments, the serotonin receptor antagonist (also referred to as 5-HT3 receptor antagonist) block serotonin receptors in the central nervous system and gastrointestinal tract. The serotonin antagonists can inhibit serotonin at the 5-HT3 receptor in the small bowel, vagus nerve, and chemoreceptor trigger zone. Specifically, 5-HT3 receptor activation induces fast excitatory postsynaptic potentials and rapid depolarization of serotonergic neurons, leading to augmentation of intracellular Ca²⁺ concentration that causes the release of different emetic neurotransmitters and/or peptides and second generation 5-HT3 receptor antagonists attenuate the first phase of cancer chemotherapy-evoked vomiting where serotonin is a major emetic player.

“Alkyl” as used herein refers to a saturated linear (i.e., unbranched) or branched univalent hydrocarbon chain or combination thereof. Particular alkyl groups are those having a designated number of carbon atoms, for example, an alkyl group having 1 to 20 carbon atoms (a “C₁-C₂₀ alkyl”), having 1 to 10 carbon atoms (a “C₁-C₁₀” alkyl), having 1 to 8 carbon atoms (a “C₁-C₈ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkenyl” as used herein refers to an unsaturated linear (i.e., unbranched) or branched univalent hydrocarbon chain or combination thereof, having at least one site of olefinic unsaturation (i.e., having at least one moiety of the formula C═C). Particular alkenyl groups are those having a designated number of carbon atoms, for example, an alkenyl group having 2 to 20 carbon atoms (a “C₂-C₂₀ alkenyl”), having 2 to 10 carbon atoms (a “C₂-C₁₀” alkenyl), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). The alkenyl group may be in cis- or trans-configurations or, alternatively, in E- or Z-configurations. Examples of alkenyl groups include, but are not limited to, groups such as ethenyl (or vinyl), prop-l-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkynyl” as used herein refers to an unsaturated linear (i.e., unbranched) or branched univalent hydrocarbon chain or combination thereof, having at least one site of acetylenic unsaturation (i.e., having at least one moiety of the formula C≡C). Particular alkynyl groups are those having a designated number of carbon atoms, for example, an alkynyl group having 2 to 20 carbon atoms (a “C₂-C₂₀ alkynyl”), having 2 to 10 carbon atoms (a “C₂-C₁₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl groups include, but are not limited to, groups such as ethynyl (or acetylenyl), prop-1-ynyl, prop-2-ynyl (or propargyl), but-1-ynyl, but-2-ynyl, but-3-ynyl, homologs, and isomers thereof, and the like.

“Alkylene” as used herein refers to the same residues as alkyl, but having bivalency, or are divalent. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”), or 1 to 3 carbon atoms (a “C₁-C₃ alkylene”). Examples of alkylene groups include, but are not limited to, groups such as methylene (—CH₂—), —CH₂CH₂—, —CH₂CH₂CH₂—, —CH₂CH₂CH₂CH₂—, and the like.

“Alkenylene” as used herein refers to the same residues as alkenyl, but having bivalency, or are divalent. Particular alkenylene groups are those having 2 to 6 carbon atoms (a “C₂-C₆ alkenylene”), 2 to 5 carbon atoms (a “C₂-C₅ alkenylene”), 2 to 4 carbon atoms (a “C₂-C₄ alkenylene”), or 2 to 3 carbon atoms (a “C₂-C₃ alkenylene”). Examples of alkylene groups include, but are not limited to, groups such as —CH═CH—, —CH═CHCH₂—, —CH═CHCH₂CH₂—, and the like.

“Alkynylene” as used herein refers to the same residues as alkynyl, but having bivalency, or are divalent. Particular alkynylene groups are those having 2 to 6 carbon atoms (a “C₂-C₆ alkynylene”), 2 to 5 carbon atoms (a “C₂-C₅ alkynylene”), 2 to 4 carbon atoms (a “C₂-C₄ alkynylene”), or 2 to 3 carbon atoms (a “C₂-C₃ alkylene”). Examples of alkynylene groups include, but are not limited to, groups such as —≡C—, —C≡CCH₂—, —C≡CCH₂CH₂—, and the like.

“Amino” refers to the group —NH₂.

“Aryl” as used herein refers to an aromatic carbocyclic group having a single ring (e.g., phenyl), or multiple condensed rings (e.g., naphthyl or anthryl) where one or more of the condensed rings may not be aromatic. Particular aryl groups are those having from 6 to 14 annular (i.e., ring) carbon atoms (a “C6-C14 aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position. Examples of aryls include, but are not limited to, groups such as phenyl, naphthyl, 1-naphthyl, 2-naphthyl, 1,2,3,4-tetrahydronaphthalen-6-yl

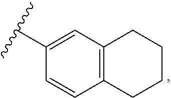 [SMILES: *c1ccc2c(c1)CCCC2]

and the like.

“Arylene” as used herein refers to the same residues as aryl, but having bivalency, or are divalent. Particular arylene groups are those having from 6 to 14 annular carbon atoms (a “C₆-C₁₄ arylene”). Examples of arylene include, but are not limited to, groups such as phenylene, o-phenylene (i.e., 1,2-phenylene), m-phenylene (i.e., 1,3-phenylene),p-phenylene (i.e., 1,4-phenylene), naphthylene, 1,2-naphthylene, 1,3-naphthylene, 1,4-naphthylene, 2,7-naphthylene, 2,6-naphthylene, and the like.

“Carbocyclyl” or “carbocyclic” refers to an aromatic or non-aromatic univalent cyclic group in which all of the ring members are carbon atoms, such as cyclohexyl, phenyl, 1,2-dihydronaphthyl, etc.

“Cycloalkyl” as used herein refers to non-aromatic, saturated or unsaturated, cyclic univalent hydrocarbon structures. Particular cycloalkyl groups are those having a designated number of annular (i.e., ring) carbon atoms, for example, a cycloalkyl group having from 3 to 12 annular carbon atoms (a “C₃-C₁₂ cycloalkyl”). A particular cycloalkyl is a cyclic hydrocarbon having from 3 to 8 annular carbon atoms (a “C₃-C₈ cycloalkyl”), or having 3 to 6 annular carbon atoms (a “C₃-C₆ cycloalkyl”). Cycloalkyl can consist of one ring, such as cyclohexyl, or multiple rings, such as adamantyl, but excludes aryl (i.e., aromatic) groups. A cycloalkyl comprising more than one ring may be fused, spiro, or bridged, or combinations thereof. Examples of cycloalkyl

groups include, but are not limited to, cyclopropyl

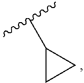 [SMILES: *C1CC1]

cyclobutyl

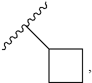 [SMILES: *C1CCC1]

cyclopentyl

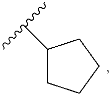 [SMILES: *C1CCCC1]

cyclohexyl

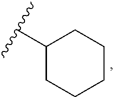 [SMILES: *C1CCCCC1]

1-cyclohexenyl, 3-cyclohexenyl, cycloheptyl

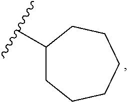 [SMILES: *C1CCCCCC1]

norbornyl, and the like.

“Cycloalkylene” as used herein refers to the same residues as cycloalkyl, but having bivalency, or are divalent. Particular cycloalkylene groups are those having 3 to 12 annular carbon atoms (a “C₃-C₁₂ cycloalkylene”), having from 3 to 8 annular carbon atoms (a “C₃-C₈ cycloalkylene”), or having 3 to 6 annular carbon atoms (a “C₃-C₆ cycloalkylene”). Examples of cycloalkylene groups include, but are not limited to, cyclopropylene

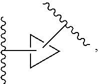

cyclobutylene

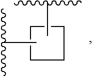

cyclopentylene

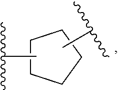

cyclohexylene

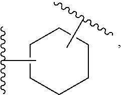

1,2-cyclohexenylene, 1,3-cyclohexenylene, 1,4-cyclohexenylene, cycloheptylene

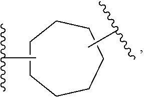

norbornylene, and the like.

“Halo” or “halogen” refers to elements of the Group 17 series having atomic number 9 to 85. Halo groups include fluoro (F), chloro (Cl), bromo (Br), and iodo (I).

“Haloalkyl,” “haloalkylene,” “haloaryl,” “haloarylene,” “haloheteroaryl,” and similar terms refer to a moiety substituted with at least one halo group. Where a haloalkyl moiety or other halo-substituted moiety is substituted with more than one halogen, it may be referred to by using a prefix corresponding to the number of halogen moieties attached. For example, dihaloaryl, dihaloalkyl, trihaloaryl, trihaloalkyl, etc., refer to aryl and alkyl substituted with two (“di”) or three (“tri”) halo groups, which may be, but are not necessarily, the same halo; thus, for example, the haloaryl group 4-chloro-3-fluorophenyl is within the scope of dihaloaryl. The subset of haloalkyl groups in which each hydrogen (H) of an alkyl group is replaced with a halo group is referred to as a “perhaloalkyl.” A particular perhaloalkyl group is trifluoroalkyl (—CF₃). Similarly, “perhaloalkoxy” refers to an alkoxy group in which a halogen takes the place of each hydrogen (H) in the hydrocarbon making up the alkyl moiety of the alkoxy group. An example of a perhaloalkoxy group is trifluoromethoxy (—OCF₃). “Haloalkyl” includes monohaloalkyl, dihaloalkyl, trihaloalkyl, perhaloalkyl, and any other number of halo substituents possible on an alkyl group; and similarly for other groups such as haloalkylene, haloaryl, haloarylene, haloheteroaryl, etc.

“Heteroaryl” as used herein refers to an unsaturated aromatic cyclic group having from 1 to 14 annular carbon atoms and at least one annular heteroatom, including, but not limited to, heteroatoms such as nitrogen (N), oxygen (O), and sulfur (S). A heteroaryl group may have a single ring (e.g., pyridyl or imidazolyl) or multiple condensed rings (e.g., indolizinyl, indolyl, or quinolinyl) where at least one of the condensed rings is aromatic. Particular heteroaryl groups are to 14-membered rings having 1 to 12 annular carbon atoms and 1 to 6 annular heteroatoms independently selected from the group consisting of nitrogen (N), oxygen (O), and sulfur (S) (a “5- to 14- membered heteroaryl”); 5- to 10-membered rings having 1 to 8 annular carbon atoms and 1 to 4 annular heteroatoms independently selected from the group consisting of nitrogen, oxygen, and sulfur (a “5- to 10- membered heteroaryl”); or 5-, 6-, or 7-membered rings having 1 to annular carbon atoms and 1 to 4 annular heteroatoms independently selected from the group consisting of nitrogen, oxygen, and sulfur (a “5- to 7- membered heteroaryl”). In one variation, heteroaryl includes monocyclic aromatic 5-, 6-, or 7-membered rings having from 1 to 6 annular carbon atoms and 1 to 4 annular heteroatoms independently selected from the group consisting of nitrogen, oxygen, and sulfur. In another variation, heteroaryl includes polycyclic aromatic rings having from 1 to 12 annular carbon atoms and 1 to 6 annular heteroatoms independently selected from the group consisting of nitrogen, oxygen, and sulfur. A heteroaryl group having more than one ring where at least one ring is non-aromatic may be connected to the parent structure at either an aromatic ring position or at a non-aromatic ring position. Examples of heteroaryl include, but are not limited to, groups such as pyridyl, benzimidazolyl, benzotriazolyl, benzo[b]thienyl, quinolinyl, indolyl, benzothiazolyl, and the like. “Heteroaryl” also includes moieties such as

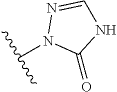 [SMILES: *n1nc[nH]c1=O]

(2,4-dihydro-3H-1,2,4-triazol-3-one-2-yl), which has the aromatic tautomeric structure

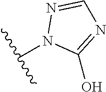 [SMILES: *n1ncnc1O]

(1H-1,2,4-triazol-5-ol-1-yl).

“Heteroarylene” as used herein refers to the same residues as heteroaryl, but having bivalency, or are divalent. Particular heteroarylene groups are 5- to 14-membered rings having 1 to 12 annular carbon atoms and 1 to 6 annular heteroatoms independently selected from the group consisting of nitrogen, oxygen, and sulfur (a “5- to 14- membered heteroarylene”); 5- to 10-membered rings having 1 to 8 annular carbon atoms and 1 to 4 annular heteroatoms independently selected from the group consisting of nitrogen, oxygen, and sulfur (a “5- to 10- membered heteroarylene”); or 5-, 6-, or 7-membered rings having 1 to 5 annular carbon atoms and 1 to 4 annular heteroatoms independently selected from the group consisting of nitrogen, oxygen, and sulfur (a “5- to 7-membered heteroarylene”). Examples of heteroarylene include, but are not limited to, groups such as pyridylene, benzimidazolylene, benzotriazolylene, benzo[b]thienylene, quinolinylene, indolylene, benzothiazolylene, and the like.

“Heterocyclyl” and “heterocyclic groups” as used herein refer to non-aromatic saturated or partially unsaturated cyclic groups having the number of atoms and heteroatoms as specified, or if no number of atoms or heteroatoms is specified, having at least three annular atoms, from 1 to 14 annular carbon atoms, and at least one annular heteroatom, including, but not limited to, heteroatoms such as nitrogen, oxygen, and sulfur. A heterocyclic group may have a single ring (e.g., tetrahydrothiopheneyl, oxazolidinyl) or multiple condensed rings (e.g., decahydroquinolinyl, octahydrobenzo[d]oxazolyl). Multiple condensed rings include, but are not limited to, bicyclic, tricyclic, and quadracylic rings, as well as bridged or spirocyclic ring systems. Examples of heterocyclic groups include, but are not limited to, aziridinyl, azetidinyl, pyrrolidinyl, piperidinyl, oxiranyl, oxetanyl, tetrahydrofuranyl, tetrahydropyranyl, oxazolidinyl, piperazinyl, morpholinyl, dioxanyl, 3,6-dihydro-2H-pyranyl, 2,3-dihydro-1H-imidazolyl, and the like.

“Oxo” refers to the group ═O(e.g., carbonyl), that is, an oxygen atom doubly bonded to carbon or another chemical element.

“Optionally substituted,” unless otherwise specified, means that a group is unsubstituted or substituted by one or more (e.g., 1, 2, 3, 4, or 5) of the substituents listed for that group, in which the substituents may be the same or different. In one embodiment, an optionally substituted group is unsubstituted. In one embodiment, an optionally substituted group has one substituent. In another embodiment, an optionally substituted group has two substituents. In another embodiment, an optionally substituted group has three substituents. In another embodiment, an optionally substituted group has four substituents. In certain embodiments, an optionally substituted group has 1 to 2, 1 to 3, 1 to 4, or 1 to 5 substituents. When multiple substituents are present, each substituent is independently chosen unless indicated otherwise. For example, each (C₁-C₄ alkyl) substituent on the group —N(C₁-C₄ alkyl)(C₁-C₄ alkyl) can be selected independently from the other, so as to generate groups such as —N(CH₃)(CH₂CH₃), etc.

The term “substituted,” when used to modify a specified group or radical, can also mean that one or more hydrogen atoms (H) of the specified group or radical are each, independently of one another, replaced with the same or different substituent groups as defined herein. In certain embodiments, a group that is substituted has 1, 2, 3, or 4 substituents, 1, 2, or 3 substituents, 1 or 2 substituents, or one substituent.

Substituents can be attached to any chemically possible location on the specified group or radical, unless indicated otherwise. Thus, in one embodiment, -C₁-C₈ alkyl-OH includes, for example, —CH₂CH₂OH, —CH(OH)—CH₃, 13 CH₂C(OH)(CH₃)₂, and the like. By way of further example, in one embodiment, -C₁-C₆ alkyl-OH includes, for example, —CH₂CH₂OH, —CH(OH)—CH₃, 13 CH₂C(OH)(CH₃)₂, and the like. By way of further example, in one embodiment, -C₁-C₆ alkyl-CN includes, for example, —CH₂CH₂CN, —CH(CN)—CH₃, —CH₂C(CN)(CH₃)₂, and the like.

Unless a specific isotope of an element is indicated in a formula, the disclosure includes all isotopologues of the compounds disclosed herein, such as, for example, deuterated derivatives of the compounds (where H can be 2 H, i.e., deuterium (D)). Deuterated compounds may provide favorable changes in pharmacokinetic (ADME) properties. Isotopologues can have isotopic replacements at any or at all locations in a structure, or can have atoms present in natural abundance at any or all locations in a structure.

A “small molecule” as used herein refers to a compound of 1,000 daltons or less in molecular weight.

Hydrogen atoms can also be replaced with bioisosteres, or close bioisosteres, such as fluorine, provided that such replacements result in stable compounds.

This disclosure also includes any or all of the stereochemical forms, including any enantiomeric or diastereomeric forms of the compounds described herein, and cis/trans or E/Z isomers. Unless stereochemistry is explicitly indicated in a chemical structure or name, the structure or name is intended to embrace all possible stereoisomers of a compound depicted. In addition, where a specific stereochemical form is depicted, it is understood that all other stereochemical forms are also described and embraced by this disclosure, as well as the general non-stereospecific form and mixtures of the disclosed compounds in any ratio, including mixtures of two or more stereochemical forms of a disclosed compound in any ratio, such that racemic, non-racemic, enantioenriched, and scalemic mixtures of a compound are embraced. Compositions comprising a disclosed compound also are intended, such as a composition of a substantially pure compound, including a specific stereochemical form thereof. Compositions comprising a mixture of disclosed compounds in any ratio also are embraced by the disclosure, including compositions comprising mixtures of two or more stereochemical forms of a disclosed compound in any ratio, such that racemic, non-racemic, enantioenriched, and scalemic mixtures of a compound are embraced by the disclosure. If stereochemistry is explicitly indicated for one portion or portions of a molecule, but not for another portion or portions of a molecule, the structure is intended to embrace all possible stereoisomers for the portion or portions where stereochemistry is not explicitly indicated.

This disclosure also embraces any and all tautomeric forms of the compounds described herein.

This disclosure is intended to embrace all salts of the compounds described herein, as well as methods of using such salts of the compounds. In one embodiment, the salts of the compounds comprise pharmaceutically acceptable salts. Pharmaceutically acceptable salts are those salts that can be administered as drugs or pharmaceuticals to humans and/or animals and that, upon administration, retain at least some of the biological activity of the free compound (i.e., neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, also can be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, also can be prepared. For lists of pharmaceutically acceptable salts, see, for example, P. H. Stahl and C. G. Wermuth (eds.) “Handbook of Pharmaceutical Salts, Properties, Selection and Use” Wiley-VCH, 2011 (ISBN: 978-3-90639-051-2). Several pharmaceutically acceptable salts are also disclosed in Berge, J. Pharm. Sci. 66:1 (1977).

Reference to a compound as described in a pharmaceutical composition, or to a compound as described in a claim to a pharmaceutical composition, refers to the compound described by the formula recited in the pharmaceutical composition, without the other elements of the pharmaceutical composition, that is, without carriers, excipients, etc.

An “effective amount” of an agent disclosed herein is an amount sufficient to carry out a specifically stated purpose. An “effective amount” may be determined empirically and in a routine manner, in relation to the stated purpose. An “effective amount” or an “amount sufficient” of an agent is that amount adequate to produce a desired biological effect, such as a beneficial result, including a beneficial clinical result. In certain embodiments, the term “effective amount” refers to an amount of an agent effective to “treat” a disease or disorder in an individual (e.g., a mammal such as a human). In certain embodiments, the term “effective amount” refers to an amount of an agent effective to treat or prevent a side effect, for instance nausea or vomiting or both, in an individual (e.g., a mammal such as a human).

“Excipients” as used herein include pharmaceutically acceptable excipients, carriers, vehicles, or stabilizers that are nontoxic to the cell or mammal being exposed thereto at the dosages and concentrations employed. Often the physiologically acceptable excipient is an aqueous pH buffered solution.

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions may be sterile. Such formulations or compositions may be sterile, with the exception of the inclusion of an oncolytic virus.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In certain embodiments, the subject has a disease that can be treated or diagnosed with an antibody, an effective amount of Cbl inhibitor compound described herein, one or more checkpoint inhibitors described herein, and combinations thereof as provided herein. In certain embodiments, the disease is gastric carcinoma, colorectal carcinoma, renal cell carcinoma, cervical carcinoma, non-small cell lung carcinoma, ovarian cancer, breast cancer, triple-negative breast cancer, endometrial cancer, prostate cancer, and/or a cancer of epithelial origin.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

### 2. Cbl Compounds

Cbl is a negative regulator of immune cell activation and is expressed in leukocytes such as lymphocytes (e.g., T-cells and NK cells) and macrophages. As such, the Cbl signaling pathway is often exploited by cancer cells to evade and limit the antitumor effector function of these immune cells. Because Cbl inhibitors work across multiple immune cell types, inhibiting the Cbl signaling pathway has the potential to reverse these effects and enhance the effector function of various immune cells.

Cbl inhibitors include small molecules, peptides, nucleic acids, or antibodies that inhibit the Cbl ligase. In the methods and compositions, the Cbl inhibitor can be any Cbl inhibitor deemed suitable to the person of skill. In certain embodiments, the Cbl inhibitor is any compound described in WO 2006/136502, WO 2007/010217, WO 2008/070305, WO 2019/148005, WO 2020/210508, WO 2020/236654, WO 2020/264398, WO 2021/021761, WO 2021/243471, WO 2022/169997, WO 2022/169998, WO 2022/217276, WO 2022/221704, WO 2022/272248, or WO 2023/036330, the contents of which are hereby incorporated by reference in their entireties. In certain embodiments, the Cbl inhibitor compound is a Cbl inhibitor compound of Formula (I):

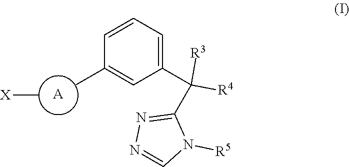

or a tautomer thereof, or a pharmaceutically acceptable salt thereof,
wherein:

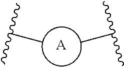

is,

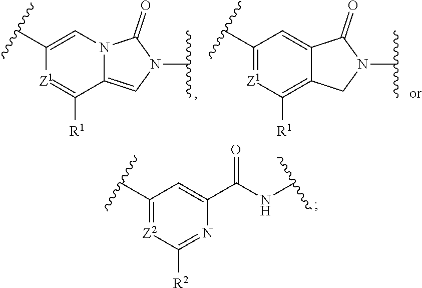

Z¹ is CH or nitrogen;
Z² is CH or nitrogen;
R¹ is —CF₃ or cyclopropyl;
R² is —CF₃ or cyclopropyl;
R³ is hydrogen, C₁-C₂ alkyl, or C₁-C₂ haloalkyl;
R⁴ is hydrogen, C₁-C₆ alkyl, C₁-C₆ haloalkyl, 4- to 8-membered heterocyclyl, or C₃-C₆ cycloalkyl, wherein the heterocyclyl or cycloalkyl groups are optionally substituted by 1-5 R⁶ groups; or R³ and R⁴ are taken together with the carbon atom to which they are attached to form C₃-C₅ cycloalkyl or 4- to 6-membered heterocyclyl, each of which is optionally substituted by 1-5
R⁶ groups;
R⁵ is hydrogen, C₁-C₆ alkyl, C₁-C₆ haloalkyl, or C₃-C₆ cycloalkyl; each R⁶ is independently C₁-C₆ alkyl, halo, hydroxy, —O—(C₁-C₆ alkyl), —CN, C₁-C₆ alkyl—CN, C₁-C₆ alkyl-OH, or C₁-C₆ haloalkyl;
or two R⁶ groups attached to the same carbon atom are taken together with the carbon atom to which they are attached to form a spiro C₃-C₆ cycloalkyl or spiro 4- to 6-membered heterocyclyl;
X is hydrogen, C₁-C₆ alkyl, C₁-C₆ haloalkyl, C₁-C₆ alkyl-OH, C₁-C₆ alkyl—CN, C₃-C₆ cycloalkyl optionally substituted by 1-5 R⁸ groups, or

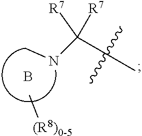

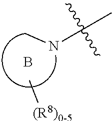

is 4- to 7-membered heterocyclyl or 5- to 8- membered heteroaryl, each of which heterocyclyl or heteroaryl optionally contains 1-2 additional heteroatoms selected from the group consisting of nitrogen, oxygen, and sulfur, and each of which heterocyclyl or heteroaryl is optionally substituted by 1-5 R⁸ groups;
each R⁷ is independently hydrogen, C₁-C₆ alkyl, C₁-C₆ alkyl-OH, or C₁-C₆ haloalkyl;
or two R⁷ groups are taken together with the carbon atom to which they are attached to form a C₃-C₅ cycloalkyl or 3- to 5- membered heterocyclyl; and
each R⁸ is independently halo, C₁-C₆ alkyl, C₁-C₆ alkyl—CN, C₁-C₆ alkyl-OH, C₁-C₆ haloalkyl, —CN, oxo, or —O(C₁-C₆ alkyl);
or two R⁸ groups are taken together with the carbon atom or atoms to which they are attached to form a spiro or fused C₃-C₅ cycloalkyl or 3- to 5-membered heterocyclyl.

In certain embodiments,

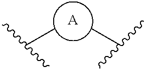

(i.e., the Ring A moiety), is

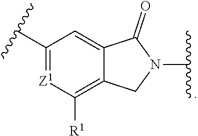

In certain embodiments,

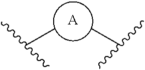

(i.e., the Ring A moiety), is

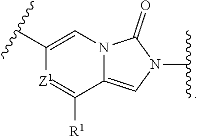

In certain embodiments, Z¹ is CH. In other embodiments, Z¹ is nitrogen. In certain embodiments, R¹ is —CF₃. In other embodiments, R¹ is cyclopropyl. In certain

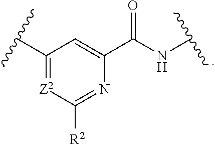

embodiments, the Ring A moiety is In certain embodiments, Z² is CH. In other embodiments, Z² is nitrogen. In certain embodiments, R² is —CF₃. In other embodiments, R² is cyclopropyl. In certain embodiments, the Ring A moiety is selected from the group consisting of:

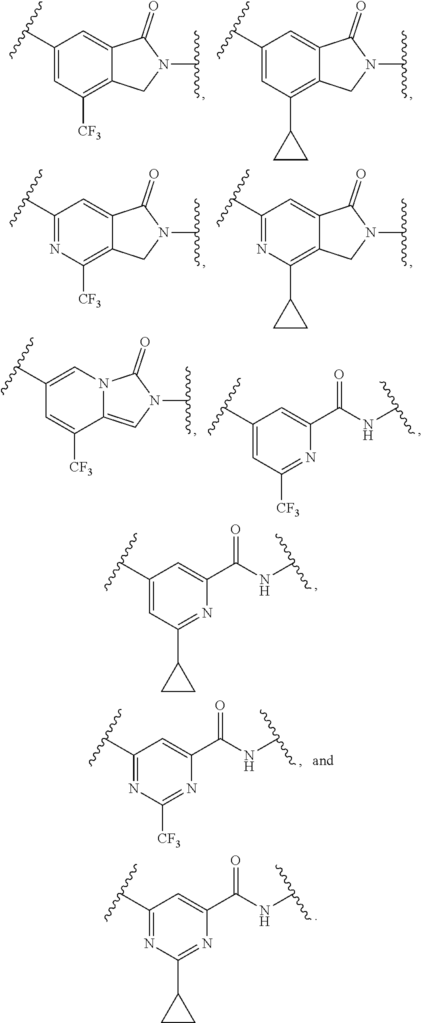

In certain embodiments, the Ring A moiety is

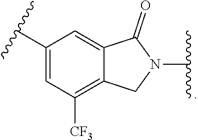 [SMILES: *c1cc2c(c(C(F)(F)F)c1)CN(*)C2=O]

In certain embodiments, the Ring A moiety is

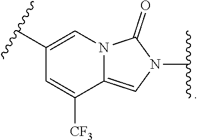 [SMILES: *c1cc(C(F)(F)F)c2cn(*)c(=O)n2c1]

In certain embodiments, the Ring A moiety is

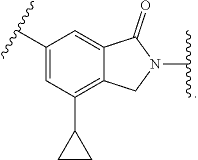 [SMILES: *c1cc2c(c(C3CC3)c1)CN(*)C2=O]

In certain embodiments, the Ring A moiety is

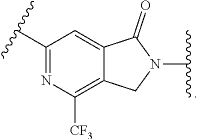 [SMILES: *c1cc2c(c(C(F)(F)F)n1)CN(*)C2=O]

In certain embodiments, the Ring A moiety is

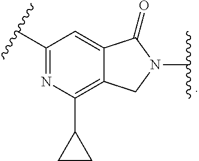 [SMILES: *c1cc2c(c(C3CC3)n1)CN(*)C2=O]

In certain embodiments, the Ring A moiety is

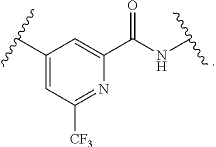 [SMILES: *NC(=O)c1cc(*)cc(C(F)(F)F)n1]

In certain embodiments, the Ring A moiety is

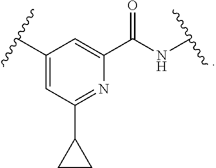 [SMILES: *NC(=O)c1cc(*)cc(C2CC2)n1]

In certain embodiments, the Ring A moiety is

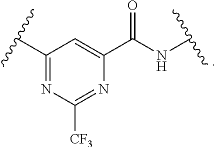 [SMILES: *NC(=O)c1cc(*)nc(C(F)(F)F)n1]

A moiety is

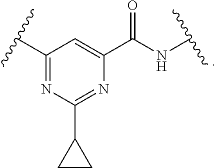 [SMILES: *NC(=O)c1cc(*)nc(C2CC2)n1]

In certain embodiments, the Ring

In certain embodiments, R³ is hydrogen, C₁-C₂ alkyl, or C₁-C₂ haloalkyl. In certain embodiments, R³ is hydrogen, —CH₃, or —CF₃.

In certain embodiments, R³ is hydrogen.

In certain embodiments, R³ is C₁-C₂ alkyl. In certain embodiments, R³ is methyl. In certain embodiments, R³ is ethyl.

In certain embodiments, R³ is C₁-C₂ haloalkyl. In certain embodiments, R³ is C₁-C₂ haloalkyl containing 1-5 halogen atoms. In certain embodiments, R³ is C₁-C₂ haloalkyl containing 1-3 halogen atoms. In certain embodiments, R³ is C₁ haloalkyl. In certain embodiments, R³ is C₂ haloalkyl. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R³ is —CF₃, —CCl₃, —CF₂C₁, -CFC12, —CHF₂, —CH₂F, —CHCl₂, —CH₂Cl, or —CHFCl. In certain embodiments, R³ is —CF₃.

In certain embodiments, R⁴ is hydrogen, C₁-C₆ alkyl, C₁-C₆ haloalkyl, 4- to 8-membered heterocyclyl, or C₃-C₆ cycloalkyl, wherein the heterocyclyl or cycloalkyl groups are optionally substituted by 1-5 R⁶ groups. In certain embodiments, R⁴ is hydrogen, C 1 -C 3 alkyl, C₁-C₃ haloalkyl, 4- to 6-membered heterocyclyl, or C₄-C₅ cycloalkyl, wherein the heterocyclyl or cycloalkyl groups are optionally substituted by 1-3 R⁶ groups. In certain embodiments, R⁴ is hydrogen, —CH₃, —CF₃, cyclobutyl, or

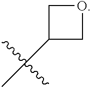 [SMILES: *C1COC1]

In certain embodiments, R⁴ is hydrogen.

In certain embodiments, R⁴ is C₁-C₆ alkyl. In certain embodiments, R⁴ is C₁-C₃ alkyl. In certain embodiments, R⁴ is methyl, ethyl, n-propyl, or isopropyl. In certain embodiments, R⁴ is —CH₃.

In certain embodiments, R⁴ is C₁-C₆ haloalkyl. In certain embodiments, R⁴ is C₁-C₆ haloalkyl containing 1-7 halogen atoms. In certain embodiments, R⁴ is C₁-C₃ haloalkyl. In certain embodiments, R⁴ is C₁-C₃ haloalkyl containing 1-7 halogen atoms. In certain embodiments, R⁴ is C₁-C₂ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁴ is —CF₃, —CCl₃, —CF₂C₁, —CFCl₂, —CHF₂, —CH₂F, —CHCl₂, —CH₂F, or —CHFC₁. In certain embodiments, R⁴ is —CF₃.

In certain embodiments, R⁴ is 4- to 8-membered heterocyclyl optionally substituted by 1-5 R⁶ groups. In certain embodiments, R⁴ is 4- to 6-membered heterocyclyl optionally substituted by 1-3 R⁶ groups. In certain embodiments, R⁴ is a 4-membered heterocyclyl optionally substituted by 1-2 R⁶ groups. In certain embodiments, the heterocyclyl is substituted by five R⁶ groups. In certain embodiments, the heterocyclyl is substituted by four R⁶ groups. In certain embodiments, the heterocyclyl is substituted by three R⁶ groups. In certain embodiments, the heterocyclyl is substituted by two R⁶ groups. In certain embodiments, the heterocyclyl is substituted by one R⁶ group. In certain embodiments, the heterocyclyl is unsubstituted. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, R⁴ is oxetanyl, azetidinyl, tetrahydrofuranyl, dioxolanyl, pyrrolidinyl, pyrazolidinyl, piperidinyl, isoxazolidinyl, or tetrahydropyranyl, each of which is optionally substituted by 1-5 R⁶ groups. In certain embodiments, R⁴ is:

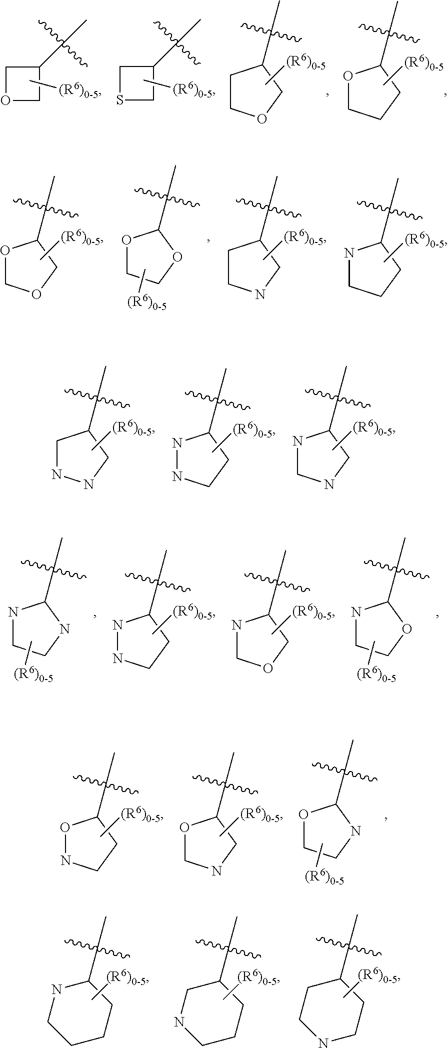

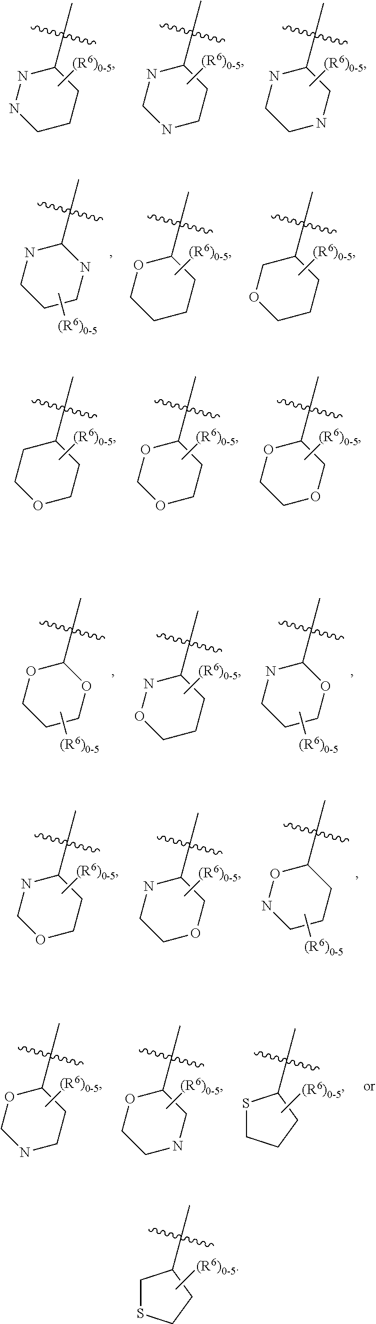

In certain embodiments, R⁴ is

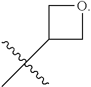 [SMILES: *C1COC1]

In certain embodiments, R⁴ is C₃-C₆ cycloalkyl optionally substituted by 1-5 R⁶ groups. In certain embodiments, R⁴ is C₄-C₅ cycloalkyl optionally substituted by 1-3 R⁶ groups. In certain embodiments, the cycloalkyl is substituted by five R⁶ groups. In certain embodiments, the cycloalkyl is substituted by four R⁶ groups. In certain embodiments, the cycloalkyl is substituted by three R⁶ groups. In certain embodiments, the cycloalkyl is substituted by two R⁶ groups. In certain embodiments, the cycloalkyl is substituted by one R⁶ group. In certain embodiments, the cycloalkyl is unsubstituted. In certain embodiments, R⁴ is cyclopropyl, cyclobutyl, cyclopentyl, or cyclohexyl, each of which is optionally substituted by 1-5 R⁶ groups. In certain embodiments, R⁴ is cyclopropyl or cyclobutyl. In certain embodiments, R⁴ is cyclobutyl.

In certain embodiments, R³ and R⁴ are taken together with the carbon atom to which they are attached to form C₃-C₅ cycloalkyl or 4- to 6-membered heterocyclyl, each of which is optionally substituted by 1-5 R⁶ groups. In certain embodiments, R³ and R⁴ are taken together with the carbon atom to which they are attached to form C₄-C₅ cycloalkyl or 4- to 6-membered heterocyclyl, each of which is optionally substituted by 1-3 R⁶ groups. In certain embodiments, R³ and R⁴ are taken together with the carbon atom to which they are attached to form

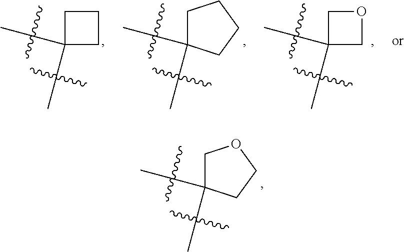 [SMILES: *C1(*)CCC1 ; *C1(*)CCCC1 ; *C1(*)COC1 ; *C1(*)CCOC1]

each of which is optionally substituted by 1-3 R⁶ groups. In certain embodiments, R³ and R⁴ are taken together with the carbon atom to which they are attached, and are substituted by one R⁶ group which is methyl, to form

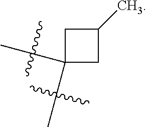 [SMILES: *C1(*)CC(C)C1]

In certain embodiments, R³ and R⁴ are taken together with the carbon atom to which they are attached to form C₃-C₅ cycloalkyl optionally substituted by 1-5 R⁶ groups. In certain embodiments, R³ and R⁴ are taken together with the carbon atom to which they are attached to form C₄-C₅ cycloalkyl optionally substituted by 1-3 R⁶ groups. In certain embodiments, the cycloalkyl is substituted by five R⁶ groups. In certain embodiments, the cycloalkyl is substituted by four R⁶ groups. In certain embodiments, the cycloalkyl is substituted by three R⁶ groups. In certain embodiments, the cycloalkyl is substituted by two R⁶ groups. In certain embodiments, the cycloalkyl is substituted by one R⁶ group. In certain embodiments, the cycloalkyl is unsubstituted. In certain embodiments, R³ and R⁴ are taken together with the carbon atom to which they are attached to form

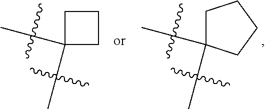 [SMILES: *C1(*)CCC1 ; *C1(*)CCCC1]

each of which is optionally substituted by 1-3 R⁶ groups. In certain embodiments, R³ and R⁴ are taken together with the carbon atom to which they are attached, and are substituted by one R⁶ group which is methyl, to form

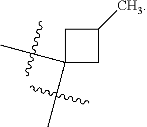 [SMILES: *C1(*)CC(C)C1]

In certain embodiments, the absolute stereochemistry at the carbon atom to which the methyl group of

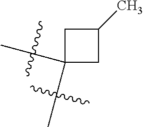 [SMILES: *C1(*)CC(C)C1]

is attached is (R)—(using the Cahn-Ingold-Prelog rules). In certain embodiments, the absolute stereochemistry at the carbon atom to which the methyl group of

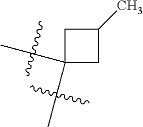 [SMILES: *C1(*)CC(C)C1]

is attached is (S)—.

In certain embodiments, R³ and R⁴ are taken together with the carbon atom to which they are attached to form 4- to 6-membered heterocyclyl optionally substituted by 1-5 R⁶ groups. In certain embodiments, R³ and R⁴ are taken together with the carbon atom to which they are attached to form 4- to 6-membered heterocyclyl optionally substituted by 1-3 R⁶ groups. In certain embodiments, the heterocyclyl is substituted by five R⁶ groups. In certain embodiments, the heterocyclyl is substituted by four R⁶ groups. In certain embodiments, the heterocyclyl is substituted by three R⁶ groups. In certain embodiments, the heterocyclyl is substituted by two R⁶ groups. In certain embodiments, the heterocyclyl is substituted by one R⁶ group. In certain embodiments, the heterocyclyl is unsubstituted. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, R⁴ is oxetanyl, azetidinyl, tetrahydrofuranyl, dioxolanyl, pyrrolidinyl, pyrazolidinyl, piperidinyl, isoxazolidinyl, or tetrahydropyranyl, each of which is optionally substituted by 1-5 R⁶ groups. In certain embodiments, R³ and R⁴ are taken together with the carbon atom to which they are attached to form

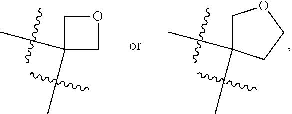 [SMILES: *C1(*)COC1 ; *C1(*)CCOC1]

each of which is optionally substituted by 1-3 R⁶ groups. In certain embodiments, R³ and R⁴ are taken together with the carbon atom to which they are attached to form

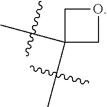 [SMILES: *C1(*)COC1]

In certain embodiments, R³ and R⁴ are taken together with the carbon atom to which they are attached to form

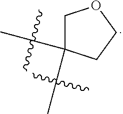 [SMILES: *C1(*)CCOC1]

In certain embodiments, each R⁶ is independently C₁-C₆ alkyl, halo, hydroxy, —O—(C₁-C₆ alkyl), —CN, C₁-C₆ alkyl—CN, C₁-C₆ alkyl-OH, or C₁-C₆ haloalkyl. In certain embodiments, each R⁶ is independently C₁-C₃ alkyl, halo, hydroxy, —O—(C₁-C₃ alkyl), —CN, C₁-C₃ alkyl—CN, C₁-C₃ alkyl-OH, or C₁-C₃ haloalkyl. In certain embodiments, each R⁶ is independently —CH₃. fluoro (F), hydroxy, —OCH₃, —CN, —CH₂CN, —CH₂OH, or —CF₃.

In certain embodiments, R⁶ is C₁-C₆ alkyl. In certain embodiments, R⁶ is C₁-C₃ alkyl. In certain embodiments, R⁶ is methyl, ethyl, n-propyl, or isopropyl. In certain embodiments, R⁶ is —CH₃.

In certain embodiments, R⁶ is halo. In certain embodiments, R⁶ is chloro, fluoro, or bromo. In certain embodiments, R⁶ is chloro or fluoro. In certain embodiments, R⁷ is fluoro.

In certain embodiments, R⁶ is hydroxyl.

In certain embodiments, R⁶ is —O(C₁-C₆ alkyl). In certain embodiments, R⁶ is —O—(C₁-C₃ alkyl). In certain embodiments, R⁶ is —O(methyl), —O(ethyl), -O(n-propyl), or —O(isopropyl). In certain embodiments, R⁶ is —OCH₃ or -OCH₂CH₃. In certain embodiments, R⁶ is —OCH₃.

In certain embodiments, R⁶ is —CN. In certain embodiments, R⁶ is C₁-C₆ alkyl—CN. In certain embodiments, R⁶ is C₁-C₃ alkyl—CN. In certain embodiments, R⁶ is —CH₂CN, —CH₂CH₂—CN, —CH₂CH₂CH₂—CN, or -C(CH₃)2—CN. In certain embodiments, R⁶ is —CH₂CN.

In certain embodiments, R⁶ is C₁-C₆ alkyl-OH. In certain embodiments, R⁶ is C₁-C₃ alkyl-OH. In certain embodiments, R⁶ is —CH₂OH, —CH₂CH₂—OH, —CH₂CH₂CH₂—OH, or —C(CH₃)₂—OH. In certain embodiments, R⁶ is —CH₂OH.

In certain embodiments, R⁶ is C₁-C₆ haloalkyl. In certain embodiments, R⁶ is C₁-C₆ haloalkyl containing 1-7 halogen atoms. In certain embodiments, R⁶ is C₁-C₃ haloalkyl. In certain embodiments, R⁶ is C₁-C₃ haloalkyl containing 1-7 halogen atoms. In certain embodiments, R⁶ is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁶ is —CF₃, —CCl₃, —CF₂C₁, —CFCl₂, —CHF₂, —CH₂F, —CHCl₂, —CH₂Cl, or —CHFC₁. In certain embodiments, R⁶ is —CF₃.

In certain embodiments, two R⁶ groups attached to the same carbon atom are taken together with the carbon atom to which they are attached to form a spiro C₃-C₆ cycloalkyl or spiro 4- to 6-membered heterocyclyl. In certain embodiments, two R⁶ groups attached to the same carbon atom are taken together with the carbon atom to which they are attached to form a spiro C₃-C₆ cycloalkyl or spiro 4- to 5-membered heterocyclyl.

In certain embodiments, two R⁶ groups attached to the same carbon atom are taken together with the carbon atom to which they are attached to form a spiro C₃-C₆ cycloalkyl. In certain embodiments, two R⁶ groups attached to the same carbon atom are taken together with the carbon atom to which they are attached to form a spiro C₃-C₅ cycloalkyl. In certain embodiments, two R⁶ groups attached to the same carbon atom are taken together with the carbon atom to which they are attached to form a spiro C₃-C₄ cycloalkyl. In certain embodiments, two R⁶ groups attached to the same carbon atom are taken together with the carbon atom to which they are attached to form a spiro cyclopropyl, cyclobutyl, or cyclopentyl. In certain embodiments, two R⁶ groups attached to the same carbon atom are taken together with the carbon atom to which they are attached to form a spiro cyclopropyl.

In certain embodiments, two R⁶ groups attached to the same carbon atom are taken together with the carbon atom to which they are attached to form a spiro 4- to 6-membered heterocyclyl. In certain embodiments, two R⁶ groups attached to the same carbon atom are taken together with the carbon atom to which they are attached to form a spiro 4- to 5-membered heterocyclyl. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, two R⁶ groups attached to the same carbon atom are taken together with the carbon atom to which they are attached to form a spiro oxetanyl, azetidinyl, tetrahydrofuranyl, dioxolanyl, pyrrolidinyl, pyrazolidinyl, piperidinyl, isoxazolidinyl, or tetrahydropyranyl.

In certain embodiments, R⁵ is hydrogen, C₁-C₆ alkyl, C₁-C₆ haloalkyl, or C₃-C₆ cycloalkyl. In certain embodiments, R⁵ is hydrogen, C₁-C₃ alkyl, C₁-C₃ haloalkyl, or C₃-C₄ cycloalkyl. In certain embodiments, R⁵ is hydrogen, —CH₃, —CHF₂, or cyclopropyl.

In certain embodiments, R⁵ is hydrogen.

In certain embodiments, R⁵ is C₁-C₆ alkyl. In certain embodiments, R⁵ is C₁-C₃ alkyl. In certain embodiments, R⁵ is methyl, ethyl, n-propyl, or isopropyl. In certain embodiments, R⁵ is —CH₃.

In certain embodiments, R⁵ is C₁-C₆ haloalkyl. In certain embodiments, R⁵ is C₁-C₆ haloalkyl containing 1-7 halogen atoms. In certain embodiments, R⁵ is C₁-C₃ haloalkyl. In certain embodiments, R⁵ is C₁-C₃ haloalkyl containing 1-7 halogen atoms. In certain embodiments, R⁵ is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁵ is —CF₃, —CCl₃, —CF₂C₁, —CFCl₂, —CHF₂, —CH₂F, —CHCl₂, —CH₂Cl, or —CHFC₁. In certain embodiments, R⁵ is —CHF₂.

In certain embodiments, R⁵ is C₃-C₆ cycloalkyl. In certain embodiments, R⁵ is C₃-C₅ cycloalkyl. In certain embodiments, R⁵ is C₃-C₄ cycloalkyl. In certain embodiments, R⁵ is cyclopropyl, cyclobutyl, or cyclopentyl. In certain embodiments, R⁵ is cyclopropyl.

In certain embodiments, X is hydrogen, C₁-C₆ alkyl, C₁-C₆ haloalkyl, C₁-C₆ alkyl-OH, C₁-C₆ alkyl—CN, or C₃-C₆ cycloalkyl optionally substituted by 1-5 R⁸ groups. In certain embodiments, X is hydrogen, C₁-C₃ alkyl, C₁-C₃ haloalkyl, C₁-C₃ alkyl-OH, C₁-C₃ alkyl—CN, or C₃-C₅ cycloalkyl optionally substituted by 1-3 R⁸ groups. In certain embodiments, X is hydrogen or —CH₃.

In certain embodiments, X is hydrogen.

In certain embodiments, X is C₁-C₆ alkyl. In certain embodiments, X is C₁-C₃ alkyl. In certain embodiments, X is methyl, ethyl, n-propyl, or isopropyl. In certain embodiments, X is —CH₃.

In certain embodiments, X is C₁-C₆ haloalkyl. In certain embodiments, X is C₁-C₆ haloalkyl containing 1-7 halogen atoms. In certain embodiments, X is C₁-C₃ haloalkyl. In certain embodiments, X is C₁-C₃ haloalkyl containing 1-7 halogen atoms. In certain embodiments, X is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, X is —CF₃, —CCl₃, —CF₂C₁, —CFCl₂, —CHF₂, —CH₂F, —CHCl₂, —CH₂Cl, or —CHFC₁. In certain embodiments, X is —CF₃.

In certain embodiments, X is C₁-C₆ alkyl-OH. In certain embodiments, X is C₁-C₃ alkyl-OH. In certain embodiments, X is —CH₂OH, —CH₂CH₂—OH, —CH₂CH₂CH₂—OH, or —C(CH₃)₂—OH. In certain embodiments, X is —CH₂OH.

In certain embodiments, X is C₁-C₆ alkyl—CN. In certain embodiments, X is C₁-C₃ alkyl—CN. In certain embodiments, X is —CH₂CN, —CH₂CH₂—CN, —CH₂CH₂CH₂—CN, or —C(CH₃)₂—CN. In certain embodiments, X is —CH₂CN.

In certain embodiments, X is C₃-C₆ cycloalkyl optionally substituted by 1-5 R⁸ groups. In certain embodiments, X is C₃-C₅ cycloalkyl optionally substituted by 1-3 R⁸ groups. In certain embodiments, the cycloalkyl is substituted by five R⁸ groups. In certain embodiments, the cycloalkyl is substituted by four R⁸ groups. In certain embodiments, the cycloalkyl is substituted by three R⁸ groups. In certain embodiments, the cycloalkyl is substituted by two R⁸ groups. In certain embodiments, the cycloalkyl is substituted by one R⁸ group. In certain embodiments, the cycloalkyl is unsubstituted. In certain embodiments, X is cyclopropyl, cyclobutyl, or cyclopentyl, each of which is optionally substituted by 1-5 R⁸ groups. In certain embodiments, X is cyclopropyl.

In certain embodiments, X is

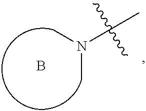

wherein the Ring B moiety, shown as

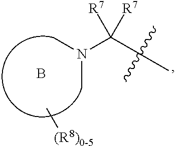

is a 4- to 7-membered heterocyclyl or 5- to 8- membered heteroaryl, each of which heterocyclyl or heteroaryl optionally contains 1-2 additional heteroatoms selected from the group consisting of nitrogen, oxygen, and sulfur, and each of which heterocyclyl or heteroaryl is optionally substituted by 1-5 R⁸ groups. In certain embodiments, the Ring B moiety is a 4- to 6-membered heterocyclyl or 5- to 6- membered heteroaryl, each of which heterocyclyl or heteroaryl optionally contains 1-2 additional heteroatoms selected from the group consisting of nitrogen, oxygen, and sulfur, and each of which heterocyclyl or heteroaryl is optionally substituted by 1-5 R⁸ groups. In certain embodiments, the Ring B moiety is a 4- to 5-membered heterocyclyl or 5- to 6- membered heteroaryl, each of which heterocyclyl or heteroaryl optionally contains one additional heteroatom selected from the group consisting of nitrogen and oxygen, and each of which heterocyclyl or heteroaryl is optionally substituted by 1-5 R⁸ groups.

In certain embodiments, the Ring B moiety is a 4- to 7-membered heterocyclyl optionally containing 1-2 additional heteroatoms selected from the group consisting of nitrogen, oxygen, and sulfur, wherein the heterocyclyl is optionally substituted by 1-5 R⁸ groups. In certain embodiments, the Ring B moiety is a 4- to 6-membered heterocyclyl optionally containing 1-2 additional heteroatoms selected from the group consisting of nitrogen, oxygen, and sulfur, wherein the heterocyclyl is optionally substituted by 1-5 R⁸ groups. In certain embodiments, the Ring B moiety is a 4- to 5-membered heterocyclyl optionally containing one additional heteroatom selected from the group consisting of nitrogen and oxygen, wherein the heterocyclyl is optionally substituted by 1-5 R⁸ groups. In certain embodiments, the heterocyclyl is substituted by five R⁸ groups. In certain embodiments, the heterocyclyl is substituted by four R⁸ groups. In certain embodiments, the heterocyclyl is substituted by three R⁸ groups. In certain embodiments, the heterocyclyl is substituted by two R⁸ groups. In certain embodiments, the heterocyclyl is substituted by one R⁸ group. In certain embodiments, the heterocyclyl is unsubstituted. In certain embodiments, the heterocyclyl contains 1-2 additional heteroatoms selected from the group consisting of nitrogen, oxygen, and sulfur. In certain embodiments, the heterocyclyl contains one additional nitrogen atom. In certain embodiments, the heterocyclyl contains two additional nitrogen atoms. In certain embodiments, the heterocyclyl further contains one oxygen atom. In certain embodiments, the heterocyclyl further contains two oxygen atoms. In certain embodiments, the heterocyclyl further contains one oxygen atom and one nitrogen atom. In certain embodiments, the heterocyclyl further contains one sulfur atom. In certain embodiments, the heterocyclyl does not contain additional heteroatoms. In certain embodiments, the heterocyclyl is azetidinyl, pyrrolidinyl, pyrazolidinyl, piperidinyl, or isoxazolidinyl, each of which is optionally substituted by 1-5 R⁸ groups.

In certain embodiments, the Ring B moiety is a 5- to 8-membered heteroaryl optionally containing 1-2 additional heteroatoms selected from the group consisting of nitrogen, oxygen, and sulfur, wherein the heteroaryl is optionally substituted by 1-5 R⁸ groups. In certain embodiments, the Ring B moiety is a 5- to 6-membered heteroaryl optionally containing 1-2 additional heteroatoms selected from the group consisting of nitrogen, oxygen, and sulfur, wherein the heteroaryl is optionally substituted by 1-5 R⁸ groups. In certain embodiments, the Ring B moiety is a 5- to 6-membered heteroaryl optionally containing one additional heteroatom selected from the group consisting of nitrogen and oxygen, wherein the heteroaryl is optionally substituted by 1-R⁸ groups. In certain embodiments, the heteroaryl is substituted by five R⁸ groups. In certain embodiments, the heteroaryl is substituted by four R⁸ groups. In certain embodiments, the heteroaryl is substituted by three R⁸ groups. In certain embodiments, the heteroaryl is substituted by two R⁸ groups. In certain embodiments, the heteroaryl is substituted by one R⁸ group. In certain embodiments, the heteroaryl is unsubstituted. In certain embodiments, the heteroaryl contains 1-2 additional heteroatoms selected from the group consisting of nitrogen, oxygen, and sulfur. In certain embodiments, the heteroaryl contains one additional nitrogen atom. In certain embodiments, the heteroaryl contains two additional nitrogen atoms. In certain embodiments, the heteroaryl further contains one oxygen atom. In certain embodiments, the heteroaryl further contains two oxygen atoms. In certain embodiments, the heteroaryl further contains one oxygen atom and one additional nitrogen atom. In certain embodiments, the heteroaryl further contains one sulfur atom. In certain embodiments, the heteroaryl does not contain additional heteroatoms. In certain embodiments, the heteroaryl is pyrrolyl, imidazolyl, pyrazolyl, isoxazolyl, oxazolyl, thiazolyl, isothiazolyl, triazolyl, pyridinyl, pyridazinyl, pyrimidinyl, pyrazinyl, or triazyl, each of which is optionally substituted by 1-5 R⁸ groups.

In certain embodiments, the Ring B moiety is

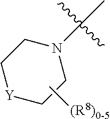

wherein Y is oxygen, —CH₂—, —CHR⁸ —, or —C(R⁸ )₂—, and X is

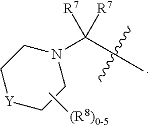

In certain embodiments, Y is oxygen (O). In other embodiments, Y is —CH₂—, —CHR⁸ —, or —C(R⁸ )₂—,. In certain embodiments, Y is —CH₂—. In certain embodiments, Y is —CHR⁸ —. In certain embodiments, Y is —C(R⁸ )2—. In certain embodiments, the Ring B moiety is substituted by a total of 1-5 R⁸ groups. In certain embodiments, the Ring B moiety is substituted by a total of 1-3 R⁸ groups. As such, if Y is -CHR⁸ -, then the Ring B moiety can be substituted by up to four additional R⁸ groups. Similarly, if Y is —C(R⁸ )₂—, then the Ring B moiety can be substituted by up to three additional R⁸ groups. In certain embodiments, the Ring B moiety is substituted by five R⁸ groups. In certain embodiments, the Ring B moiety is substituted by four R⁸ groups. In certain embodiments, the Ring B moiety is substituted by three R⁸ groups. In certain embodiments, the Ring B moiety is substituted by two R⁸ groups. In certain embodiments, the Ring B moiety is substituted by one R⁸ group. In certain embodiments, the Ring B moiety is unsubstituted. In certain embodiments, the Ring B moiety is

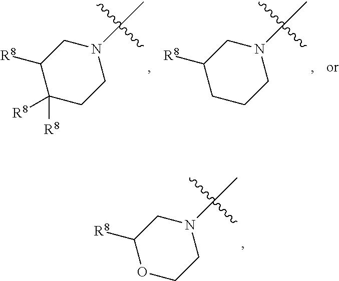

wherein each R⁸ is independently as described herein.

In certain embodiments, each R⁷ is independently hydrogen, C₁-C₆ alkyl, C₁-C₆ alkyl-OH, or C₁-C₆ haloalkyl. In certain embodiments, each R⁷ is independently hydrogen, C₁-C₃ alkyl, C₁-C₃ alkyl-OH, or C₁-C₃ haloalkyl. In certain embodiments, each R⁷ is independently hydrogen, —CH₃, —CH₂OH, or —CF₃.

In certain embodiments, both R⁷ groups are hydrogen (H). In certain embodiments, one R⁷ group is hydrogen. In certain embodiments, one R⁷ group is hydrogen, and the other R⁷ group is C₁-C₆ alkyl, C₁-C₆ alkyl-OH, or C₁-C₆ haloalkyl. In certain embodiments, one R⁷ group is hydrogen and the other R⁷ group is C₁-C₆ alkyl. In certain embodiments, one R⁷ group is hydrogen and the other R⁷ group is C₁-C₃ alkyl. In certain embodiments, one R⁷ group is hydrogen and the other R⁷ group is —CH₃.

In certain embodiments, R⁷ is C₁-C₆ alkyl. In certain embodiments, R⁷ is C₁-C₃ alkyl. In certain embodiments, R⁷ is methyl, ethyl, n-propyl, or isopropyl. In certain embodiments, one R⁷ group is methyl, ethyl, n-propyl, or isopropyl, and the other R⁷ group is hydrogen. In certain embodiments, R⁷ is —CH₃.

In certain embodiments, R⁷ is C₁-C₆ alkyl-OH. In certain embodiments, R⁷ is C₁-C₃ alkyl-OH. In certain embodiments, R⁷ is —CH₂OH, —CH₂CH₂—OH, —CH₂CH₂CH₂—OH, or —C(CH₃)₂-OH. In certain embodiments, R⁷ is —CH₂OH. In certain embodiments, one R⁷ group is C₁-C₆ alkyl-OH, and the other R⁷ group is hydrogen. In certain embodiments, one R⁷ group is C₁-C₃ alkyl-OH, and the other R⁷ group is hydrogen. In certain embodiments, one R⁷ group is —CH₂OH, —CH₂CH₂—OH, —CH₂CH₂CH₂—OH, or —C(CH₃)₂-OH, and the other R⁷ group is hydrogen. In certain embodiments, one R⁷ group is —CH₂OH, and the other R⁷ group is hydrogen.

In certain embodiments, R⁷ is C₁-C₆ haloalkyl. In certain embodiments, R⁷ is C₁-C₆ haloalkyl containing 1-7 halogen atoms. In certain embodiments, R⁷ is C₁-C₃ haloalkyl. In certain embodiments, R⁷ is C₁-C₃ haloalkyl containing 1-7 halogen atoms. In certain embodiments, R⁷ is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁷ is —CF₃, —CCl₃, —CF₂C₁, —CFCl₂, —CHF₂, —CH₂F, —CHCl₂, —CH₂F, or —CHFC₁. In certain embodiments, R⁷ is —CF₃.

In certain embodiments, two R⁷ groups are taken together with the carbon atom to which they are attached to form a C₃-C₅ cycloalkyl or 3- to 5- membered heterocyclyl. In certain embodiments, two R⁷ groups are taken together with the carbon atom to which they are attached to form cyclopropyl or oxetanyl.

In certain embodiments, two R⁷ groups are taken together with the carbon atom to which they are attached to form a C₃-C₅ cycloalkyl. In certain embodiments, two R⁷ groups are taken together with the carbon atom to which they are attached to form cyclopropyl or cyclobutyl. In certain embodiments, two R⁷ groups are taken together with the carbon atom to which they are attached to form cyclopropyl.

In certain embodiments, two R⁷ groups are taken together with the carbon atom to which they are attached to form a 3- to 5- membered heterocyclyl. In certain embodiments, two R⁷ groups are taken together with the carbon atom to which they are attached to form a 3- to 4-membered heterocyclyl. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, R⁷ is aziridinyl, oxiranyl, oxetanyl, azetidinyl, tetrahydrofuranyl, dioxolanyl, pyrrolidinyl, pyrazolidinyl, or isoxazolidinyl.

In certain embodiments, each R⁸ is independently halo, C₁-C₆ alkyl, C₁-C₆ alkyl—CN, C₁-C₆ alkyl-OH, C₁-C₆ haloalkyl, —CN, oxo, or —O(C₁-C₆ alkyl). In certain embodiments, each R⁸ is independently halo, C₁-C₃ alkyl, C₁-C₃ alkyl—CN, C₁-C₃ alkyl-OH, C₁-C₃ haloalkyl, —CN, oxo, or

—O(C₁-C₃ alkyl). In certain embodiments, each R⁸ is independently fluoro (F), —CH₃.
—CH₂CH3, —CH₂CN, —CH₂OH, —CF₃, —CN, oxo, or —OCH₃.

In certain embodiments, R⁸ is halo. In certain embodiments, R⁸ is chloro, fluoro, or bromo. In certain embodiments, R⁸ is chloro or fluoro. In certain embodiments, R⁸ is fluoro.

In certain embodiments, R⁸ is C₁-C₆ alkyl. In certain embodiments, R⁸ is C₁-C₃ alkyl. In certain embodiments, R⁸ is methyl, ethyl, n-propyl, or isopropyl. In certain embodiments, R⁸ is —CH₃ or —CH₂CH₃.

In certain embodiments, R⁸ is —CN. In certain embodiments, R⁸ is C₁-C₆ alkyl—CN. In certain embodiments, R⁸ is C₁-C₃ alkyl—CN. In certain embodiments, R⁸ is —CH₂CN, —CH₂CH₂—CN, —CH₂CH₂CH₂—CN, or —C(CH₃)₂—CN. In certain embodiments, R⁸ is —CH₂CN.

In certain embodiments, R⁸ is C₁-C₆ alkyl-OH. In certain embodiments, R⁸ is C₁-C₃ alkyl-OH. In certain embodiments, R⁸ is —CH₂OH, —CH₂CH₂—OH, —CH₂CH₂CH₂—OH, or —C(CH₃)₂-OH. In certain embodiments, R⁸ is —CH₂OH.

In certain embodiments, R⁸ is C₁-C₆ haloalkyl. In certain embodiments, R⁸ is C₁-C₆ haloalkyl containing 1-7 halogen atoms. In certain embodiments, R⁸ is C₁-C₃ haloalkyl. In certain embodiments, R⁸ is C₁-C₃ haloalkyl containing 1-7 halogen atoms. In certain embodiments, R⁸ is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁸ is —CF₃, —CCl₃, —CF₂C₁, —CFCl₂, —CHF₂, —CH₂F, —CHCl₂, —CH₂F, or —CHFC₁. In certain embodiments, R⁸ is —CF₃.

In certain embodiments, R⁸ is oxo.

In certain embodiments, R⁸ is —O(C₁-C₆ alkyl). In certain embodiments, R⁸ is —O—(C₁-C₃ alkyl). In certain embodiments, R⁸ is —O(methyl), —O(ethyl), —O(n-propyl), or —O(isopropyl). In certain embodiments, R⁸ is —OCH₃ or —OCH₂CH₃. In certain embodiments, R⁸ is —OCH₃.

In certain embodiments, two R⁸ groups are taken together with the carbon atom or atoms to which they are attached to form a spiro or fused C₃-C₅ cycloalkyl or 3- to 5-membered heterocyclyl. In certain embodiments, two R⁸ groups are taken together with the carbon atom or atoms to which they are attached to form a spiro or fused cyclopropyl or oxetanyl.

In certain embodiments, two R⁸ groups are taken together with the carbon atom or atoms to which they are attached to form a spiro or fused C₃-C₅ cycloalkyl. In certain embodiments, two R⁸ groups are taken together with the carbon atom or atoms to which they are attached to form a spiro C₃-C₅ cycloalkyl. In certain embodiments, two R⁸ groups are taken together with the carbon atom or atoms to which they are attached to form a spiro cyclopropyl. In certain embodiments, two R⁸ groups are taken together with the carbon atom or atoms to which they are attached to form a spiro cyclobutyl. In certain embodiments, two R⁸ groups are taken together with the carbon atom or atoms to which they are attached to form a spiro cyclopentyl. In certain embodiments, two R⁸ groups are taken together with the carbon atom or atoms to which they are attached to form a fused C₃-C₅ cycloalkyl. In certain embodiments, two R⁸ groups are taken together with the carbon atom or atoms to which they are attached to form a fused cyclopropyl. In certain embodiments, two R⁸ groups are taken together with the carbon atom or atoms to which they are attached to form a fused cyclobutyl. In certain embodiments, two R⁸ groups are taken together with the carbon atom or atoms to which they are attached to form a fused cyclopentyl. In certain embodiments, two R⁸ groups are taken together with the carbon atom or atoms to which they are attached to form a spiro or fused cyclopropyl.

In certain embodiments, two R⁸ groups are taken together with the carbon atom or atoms to which they are attached to form a spiro or fused 3- to 5-membered heterocyclyl. In certain embodiments, two R⁸ groups are taken together with the carbon atom or atoms to which they are attached to form a spiro 3- to 5-membered heterocyclyl. In certain embodiments, two R⁸ groups are taken together with the carbon atom or atoms to which they are attached to form a spiro oxetanyl. In certain embodiments, two R⁸ groups are taken together with the carbon atom or atoms to which they are attached to form a fused 3- to 5-membered heterocyclyl. In certain embodiments, two R⁸ groups are taken together with the carbon atom or atoms to which they are attached to form a fused oxetanyl. In certain embodiments, two R⁸ groups are taken together with the carbon atom or atoms to which they are attached to form a spiro or fused oxetanyl. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, two R⁸ groups are taken together with the carbon atom to which they are attached to form a spiro aziridinyl, oxiranyl, oxetanyl, azetidinyl, tetrahydrofuranyl, dioxolanyl, pyrrolidinyl, pyrazolidinyl, or isoxazolidinyl. In certain embodiments, two R⁸ groups are taken together with the carbon atoms to which they are attached to form a fused aziridinyl, oxiranyl, oxetanyl, azetidinyl, tetrahydrofuranyl, dioxolanyl, pyrrolidinyl, pyrazolidinyl, or isoxazolidinyl.

In certain embodiments, X is

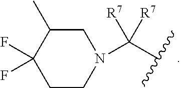

In certain embodiments, X is

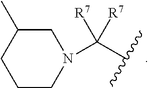

In certain embodiments, X is

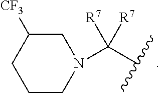

In certain embodiments, X is

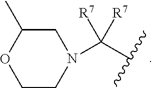

In certain embodiments, X is

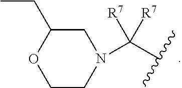

In certain embodiments, X is

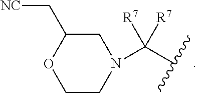

In any of these embodiments, both R⁷ groups can be hydrogen (H). In any of these embodiments, one R⁷ group can be hydrogen and one R⁷ group can be —CH₃. In any of these embodiments, both R⁷ groups can be —CH₃.

In certain embodiments, the compound is of Formula (I-A) or (I-B):

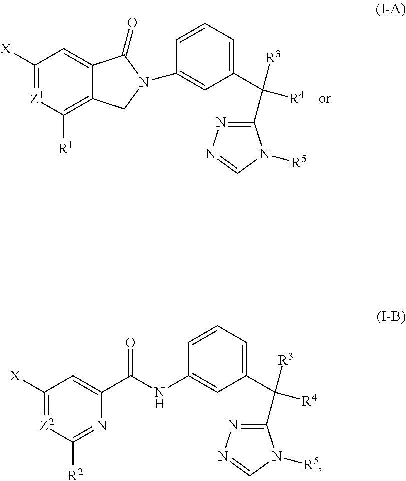

wherein R¹, R², R³, R⁴, R⁵, Z¹, Z², and X are as described for the compound of Formula (I).

In certain embodiments, the compound is of Formula (I-a) or (I-b):

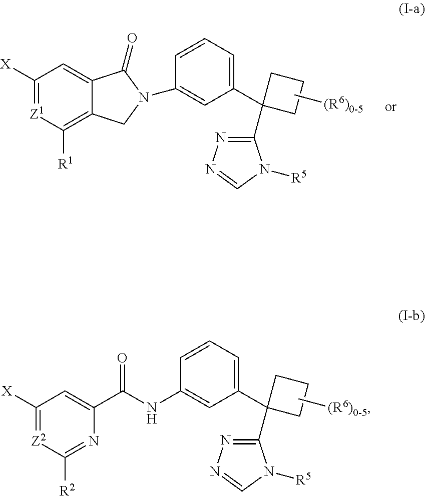

wherein R¹, R², R⁵, R⁶, Z¹, Z², and X are as described for the compound of Formula (I).

In certain embodiments, the compound is of Formula (I-C), (I-D), (I-E), (I-F), (I-G), (I-H), or (I-J):

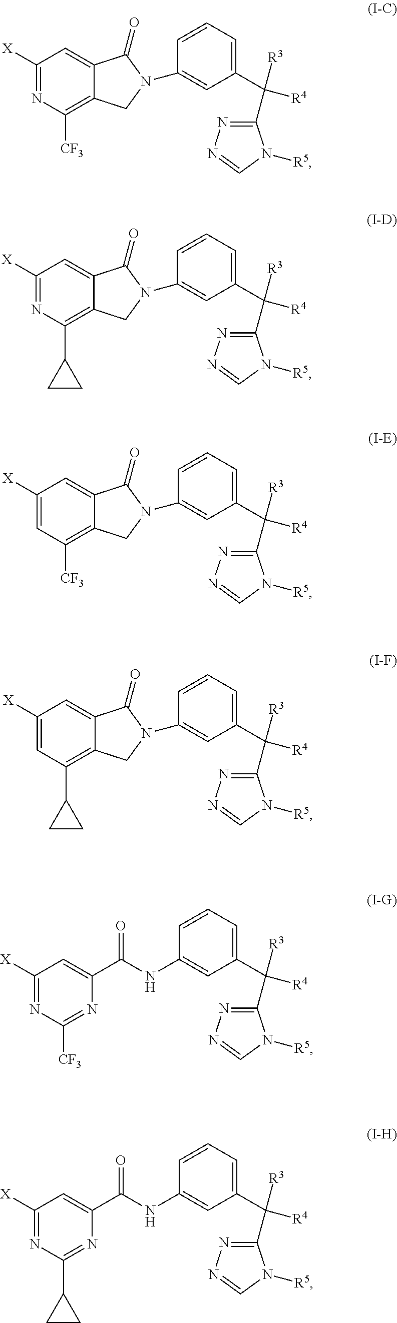

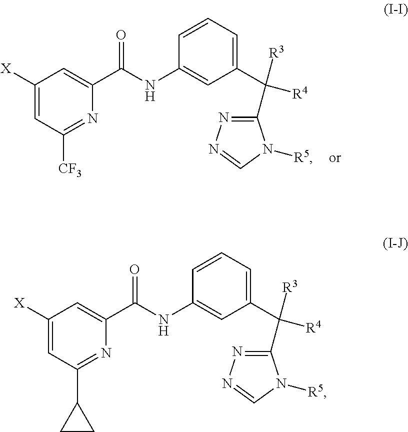

wherein R³, R⁴, R⁵, and X are as described for the compound of Formula (I).

In certain embodiments, the compound is of Formula (II-A), (II-B), (II-C), (II-D), (II-E), (II-F), (II-G), or (II-H):

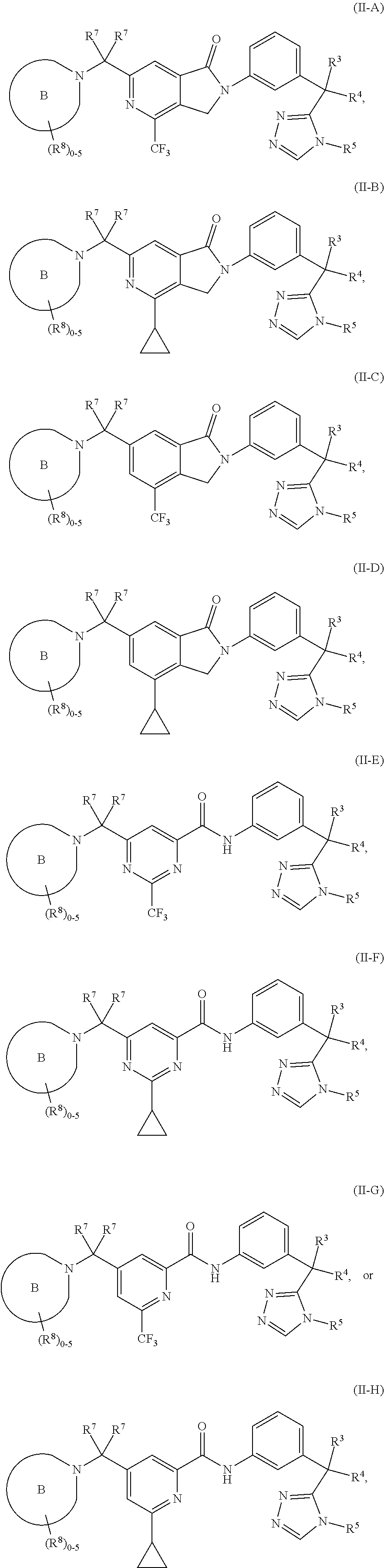

wherein R³, R⁴, R⁵, R⁷, R⁸, and the Ring B moiety are as described for the compound of Formula (I).

In certain embodiments, the compound is of Formula (III-A), (III-B), (III-C), (III-D), (III-E), (III-F), (III-G), or (III-H):

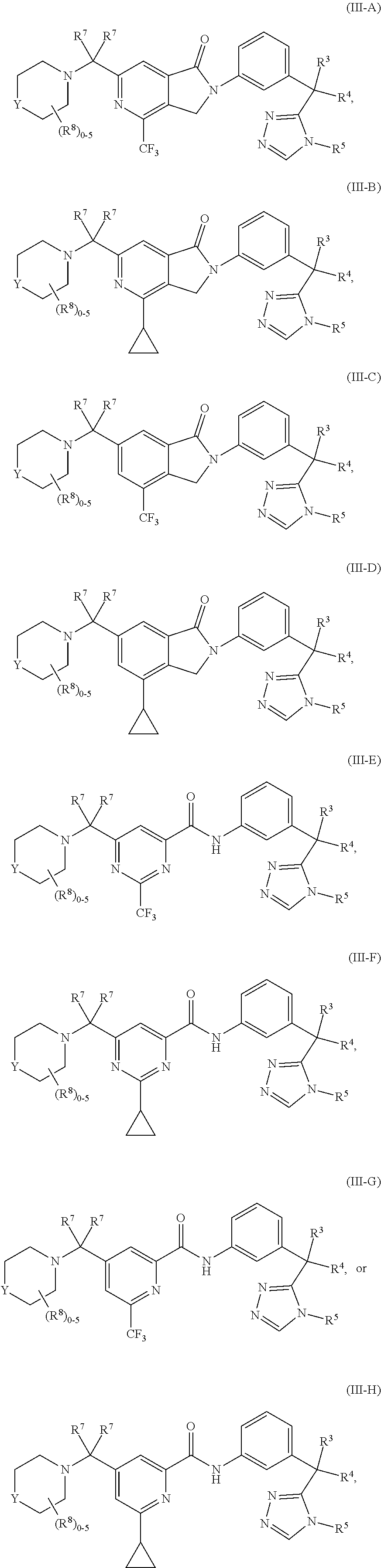

wherein R³, R⁴, R⁵, R⁷, R⁸, and Y are as described for the compound of Formula (I).

In certain embodiments, the compound is of Formula (IV-A), (IV-B), (IV-C), (IV-D), (IV-E), (IV-F), (IV-G), or (IV-H):

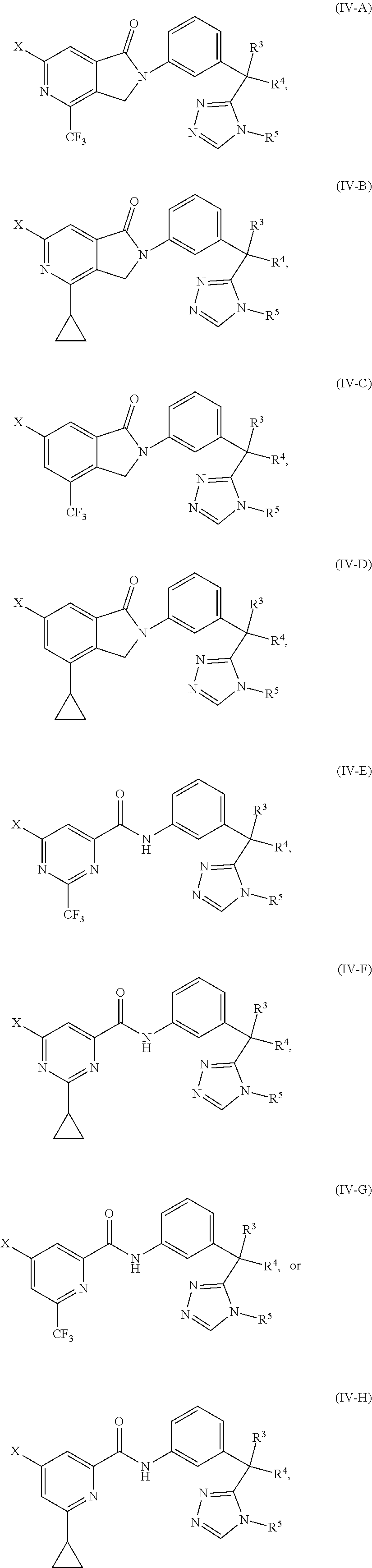

wherein R³, R⁴, and R⁵ are as described for the compound of Formula (I), and X is hydrogen, C₁-C₆ alkyl, C₁-C₆ haloalkyl, C₁-C₆ alkyl-OH, C₁-C₆ alkyl—CN, or C₃-C₆ cycloalkyl optionally substituted by 1-5 R⁸ groups. In certain embodiments, X is hydrogen. In certain embodiments, X is C₁-C₆ alkyl. In certain embodiments, X is C₁-C₆ haloalkyl. In certain embodiments, X is C₁-C₆ alkyl-OH. In certain embodiments, X is C₁-C₆ alkyl—CN. In certain embodiments, X is C₃-C₆ cycloalkyl optionally substituted by 1-5 R⁸ groups.

In certain embodiments, the compound is from Table 1, or a pharmaceutically acceptable stereoisomer, tautomer, or salt thereof.

TABLE 1
Representative Compounds of This Disclosure

| Cmpd No. | Structure |
|---|---|
| 1 | 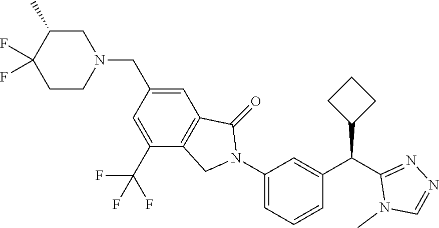 [SMILES: C[C@@H]1CN(Cc2cc3c(c(C(F)(F)F)c2)CN(c2cccc([C@H](c4nncn4C)C4CCC4)c2)C3=O)CCC1(F)F] |
| 2 | 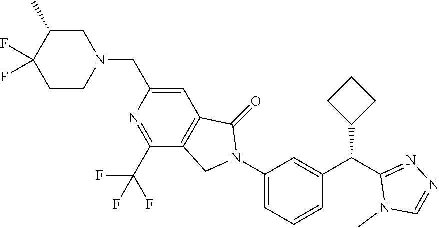 [SMILES: C[C@@H]1CN(Cc2cc3c(c(C(F)(F)F)n2)CN(c2cccc([C@@H](c4nncn4C)C4CCC4)c2)C3=O)CCC1(F)F] |
| 3 | 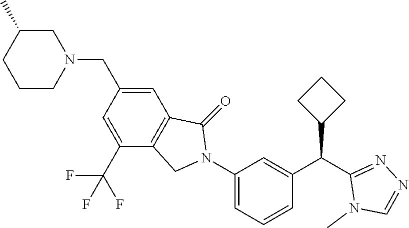 [SMILES: C[C@H]1CCCN(Cc2cc3c(c(C(F)(F)F)c2)CN(c2cccc([C@H](c4nncn4C)C4CCC4)c2)C3=O)C1] |
| 4 | 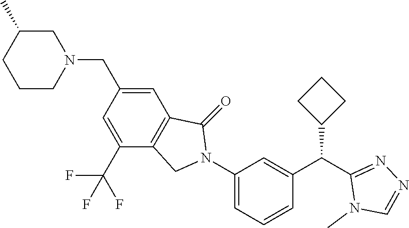 [SMILES: C[C@H]1CCCN(Cc2cc3c(c(C(F)(F)F)c2)CN(c2cccc([C@@H](c4nncn4C)C4CCC4)c2)C3=O)C1] |
| 5 | 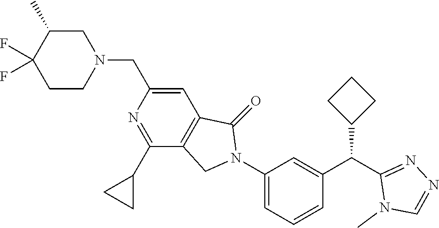 [SMILES: C[C@@H]1CN(Cc2cc3c(c(C4CC4)n2)CN(c2cccc([C@@H](c4nncn4C)C4CCC4)c2)C3=O)CCC1(F)F] |
| 6 | 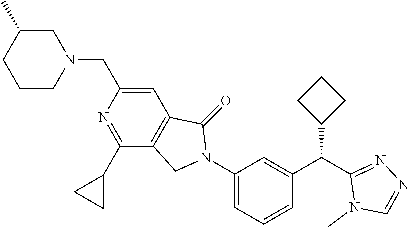 [SMILES: C[C@H]1CCCN(Cc2cc3c(c(C4CC4)n2)CN(c2cccc([C@@H](c4nncn4C)C4CCC4)c2)C3=O)C1] |
| 7 | 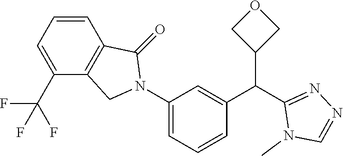 [SMILES: Cn1cnnc1C(c1cccc(N2Cc3c(cccc3C(F)(F)F)C2=O)c1)C1COC1] |
| 8 | 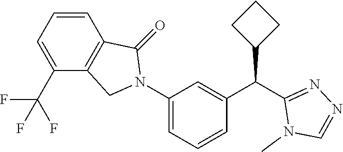 [SMILES: Cn1cnnc1[C@@H](c1cccc(N2Cc3c(cccc3C(F)(F)F)C2=O)c1)C1CCC1] |
| 9 | 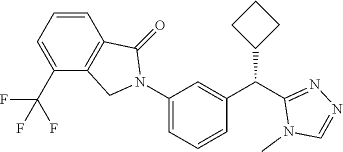 [SMILES: Cn1cnnc1[C@H](c1cccc(N2Cc3c(cccc3C(F)(F)F)C2=O)c1)C1CCC1] |
| 10 | 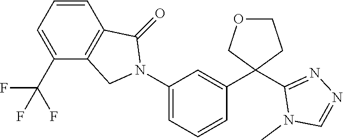 [SMILES: Cn1cnnc1C1(c2cccc(N3Cc4c(cccc4C(F)(F)F)C3=O)c2)CCOC1] |
| 11 | 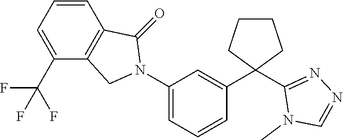 [SMILES: Cn1cnnc1C1(c2cccc(N3Cc4c(cccc4C(F)(F)F)C3=O)c2)CCCC1] |
| 12 | 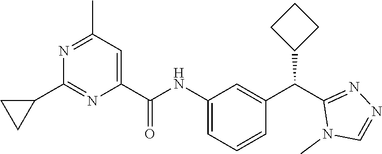 [SMILES: Cc1cc(C(=O)Nc2cccc([C@@H](c3nncn3C)C3CCC3)c2)nc(C2CC2)n1] |
| 13 | 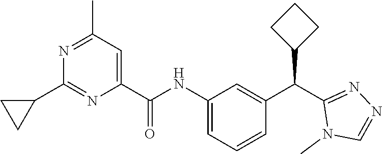 [SMILES: Cc1cc(C(=O)Nc2cccc([C@H](c3nncn3C)C3CCC3)c2)nc(C2CC2)n1] |
| 14 | 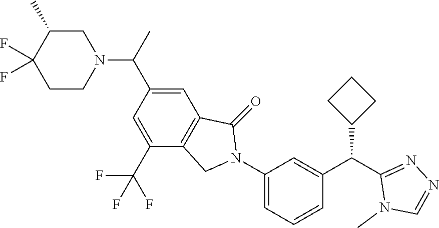 [SMILES: CC(c1cc2c(c(C(F)(F)F)c1)CN(c1cccc([C@@H](c3nncn3C)C3CCC3)c1)C2=O)N1CCC(F)(F)[C@H](C)C1] |
| 15 | 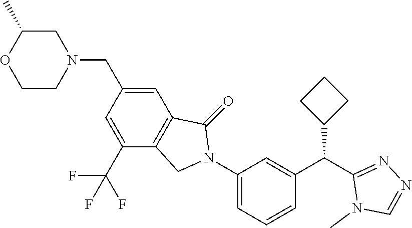 [SMILES: C[C@@H]1CN(Cc2cc3c(c(C(F)(F)F)c2)CN(c2cccc([C@@H](c4nncn4C)C4CCC4)c2)C3=O)CCO1] |
| 16 | 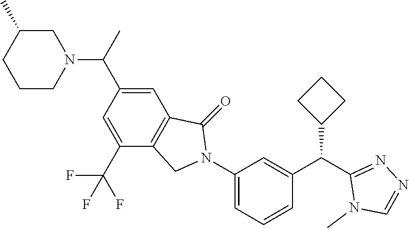 [SMILES: CC(c1cc2c(c(C(F)(F)F)c1)CN(c1cccc([C@@H](c3nncn3C)C3CCC3)c1)C2=O)N1CCC[C@H](C)C1] |
| 17 | 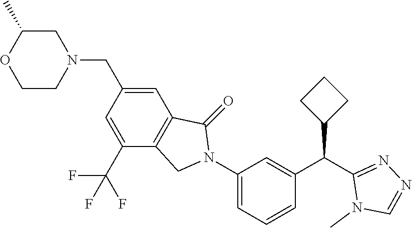 [SMILES: C[C@@H]1CN(Cc2cc3c(c(C(F)(F)F)c2)CN(c2cccc([C@H](c4nncn4C)C4CCC4)c2)C3=O)CCO1] |
| 18 | 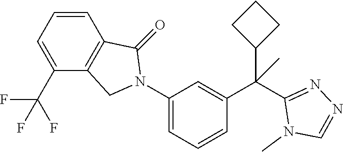 [SMILES: Cn1cnnc1C(C)(c1cccc(N2Cc3c(cccc3C(F)(F)F)C2=O)c1)C1CCC1] |
| 19 | 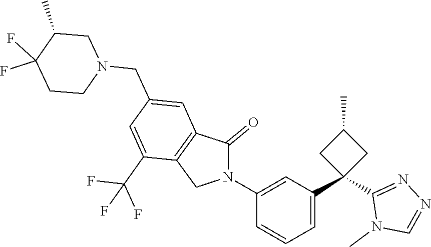 [SMILES: C[C@@H]1CN(Cc2cc3c(c(C(F)(F)F)c2)CN(c2cccc([C@]4(c5nncn5C)C[C@@H](C)C4)c2)C3=O)CCC1(F)F] |
| 20 | 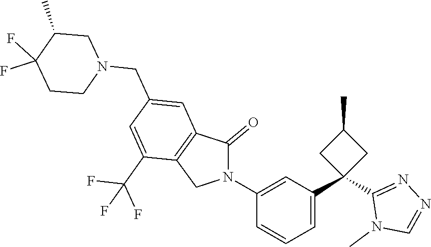 [SMILES: C[C@@H]1CN(Cc2cc3c(c(C(F)(F)F)c2)CN(c2cccc([C@]4(c5nncn5C)C[C@H](C)C4)c2)C3=O)CCC1(F)F] |
| 21 | 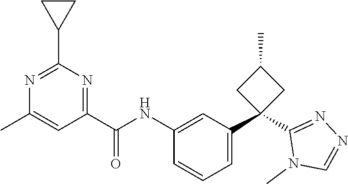 [SMILES: Cc1cc(C(=O)Nc2cccc([C@]3(c4nncn4C)C[C@@H](C)C3)c2)nc(C2CC2)n1] |
| 22 | 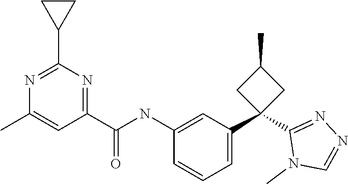 [SMILES: Cc1cc(C(=O)Nc2cccc([C@]3(c4nncn4C)C[C@H](C)C3)c2)nc(C2CC2)n1] |
| 23 | 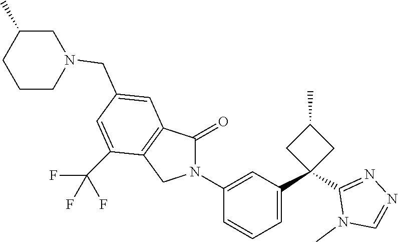 [SMILES: C[C@H]1CCCN(Cc2cc3c(c(C(F)(F)F)c2)CN(c2cccc([C@]4(c5nncn5C)C[C@@H](C)C4)c2)C3=O)C1] |
| 24 | 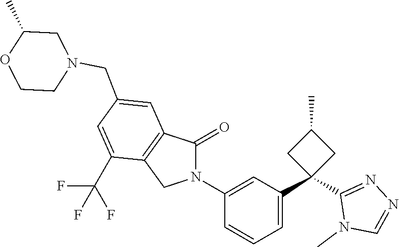 [SMILES: C[C@@H]1CN(Cc2cc3c(c(C(F)(F)F)c2)CN(c2cccc([C@]4(c5nncn5C)C[C@@H](C)C4)c2)C3=O)CCO1] |
| 25 | 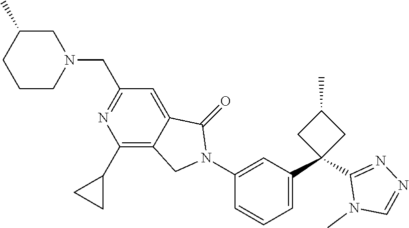 [SMILES: C[C@H]1CCCN(Cc2cc3c(c(C4CC4)n2)CN(c2cccc([C@]4(c5nncn5C)C[C@@H](C)C4)c2)C3=O)C1] |
| 26 | 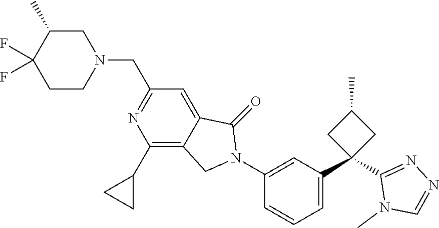 [SMILES: C[C@@H]1CN(Cc2cc3c(c(C4CC4)n2)CN(c2cccc([C@]4(c5nncn5C)C[C@@H](C)C4)c2)C3=O)CCC1(F)F] |
| 27 | 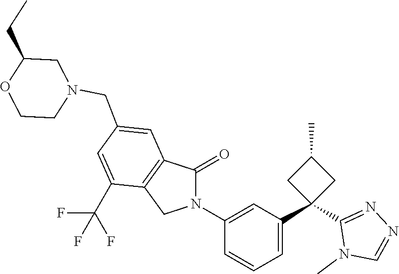 [SMILES: CC[C@H]1CN(Cc2cc3c(c(C(F)(F)F)c2)CN(c2cccc([C@]4(c5nncn5C)C[C@@H](C)C4)c2)C3=O)CCO1] |
| 28 | 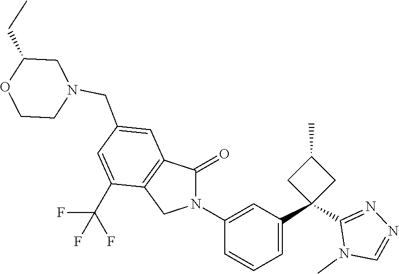 [SMILES: CC[C@@H]1CN(Cc2cc3c(c(C(F)(F)F)c2)CN(c2cccc([C@]4(c5nncn5C)C[C@@H](C)C4)c2)C3=O)CCO1] |
| 29 | 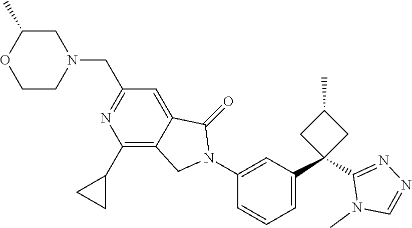 [SMILES: C[C@@H]1CN(Cc2cc3c(c(C4CC4)n2)CN(c2cccc([C@]4(c5nncn5C)C[C@@H](C)C4)c2)C3=O)CCO1] |
| 30 | 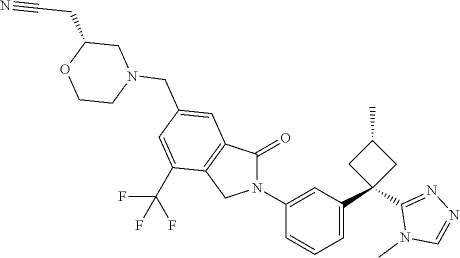 [SMILES: Cn1cnnc1[C@]1(c2cccc(N3Cc4c(cc(CN5CCO[C@H](CC#N)C5)cc4C(F)(F)F)C3=O)c2)C[C@H](C)C1] |
| 31 | 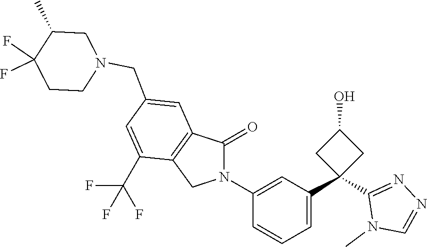 [SMILES: C[C@@H]1CN(Cc2cc3c(c(C(F)(F)F)c2)CN(c2cccc([C@]4(c5nncn5C)C[C@@H](O)C4)c2)C3=O)CCC1(F)F] |
| 32 | 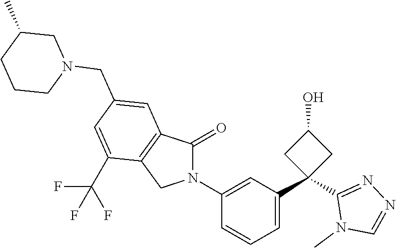 [SMILES: C[C@H]1CCCN(Cc2cc3c(c(C(F)(F)F)c2)CN(c2cccc([C@]4(c5nncn5C)C[C@@H](O)C4)c2)C3=O)C1] |
| 33 | 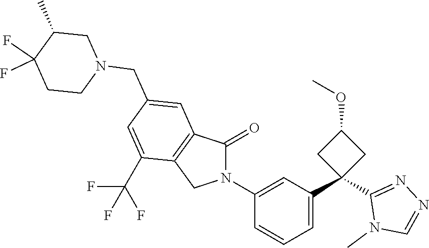 [SMILES: CO[C@H]1C[C@@](c2cccc(N3Cc4c(cc(CN5CCC(F)(F)[C@H](C)C5)cc4C(F)(F)F)C3=O)c2)(c2nncn2C)C1] |
| 34 | 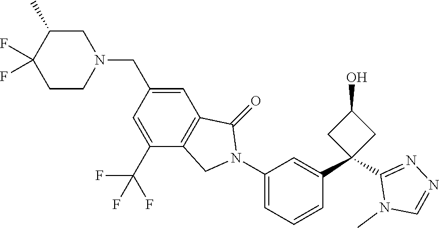 [SMILES: C[C@@H]1CN(Cc2cc3c(c(C(F)(F)F)c2)CN(c2cccc([C@]4(c5nncn5C)C[C@H](O)C4)c2)C3=O)CCC1(F)F] |
| 35 | 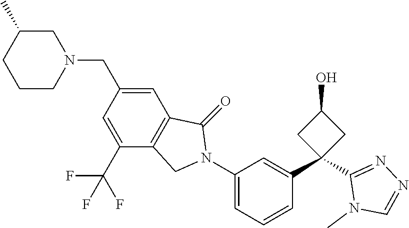 [SMILES: C[C@H]1CCCN(Cc2cc3c(c(C(F)(F)F)c2)CN(c2cccc([C@]4(c5nncn5C)C[C@H](O)C4)c2)C3=O)C1] |
| 36 | 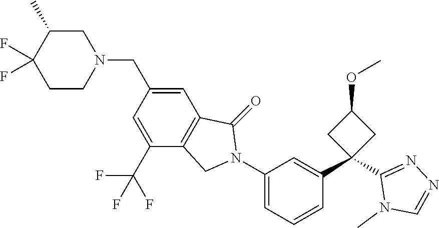 [SMILES: CO[C@H]1C[C@](c2cccc(N3Cc4c(cc(CN5CCC(F)(F)[C@H](C)C5)cc4C(F)(F)F)C3=O)c2)(c2nncn2C)C1] |
| 37 | 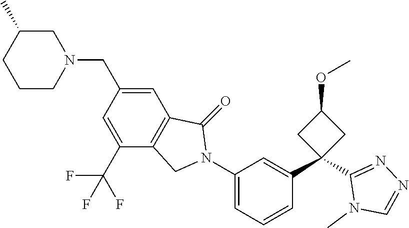 [SMILES: CO[C@H]1C[C@](c2cccc(N3Cc4c(cc(CN5CCC[C@H](C)C5)cc4C(F)(F)F)C3=O)c2)(c2nncn2C)C1] |
| 38 | 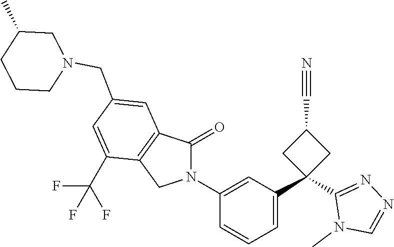 [SMILES: C[C@H]1CCCN(Cc2cc3c(c(C(F)(F)F)c2)CN(c2cccc([C@]4(c5nncn5C)C[C@@H](C#N)C4)c2)C3=O)C1] |
| 39 | 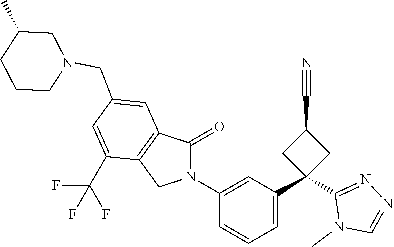 [SMILES: C[C@H]1CCCN(Cc2cc3c(c(C(F)(F)F)c2)CN(c2cccc([C@]4(c5nncn5C)C[C@H](C#N)C4)c2)C3=O)C1] |
| 40 | 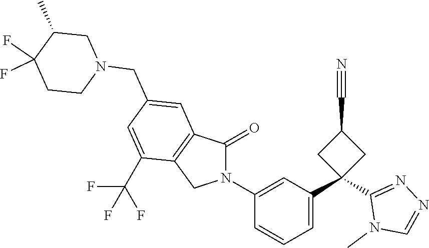 [SMILES: C[C@@H]1CN(Cc2cc3c(c(C(F)(F)F)c2)CN(c2cccc([C@]4(c5nncn5C)C[C@H](C#N)C4)c2)C3=O)CCC1(F)F] |
| 41 | 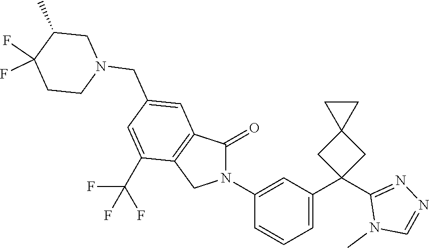 [SMILES: C[C@@H]1CN(Cc2cc3c(c(C(F)(F)F)c2)CN(c2cccc(C4(c5nncn5C)CC5(CC5)C4)c2)C3=O)CCC1(F)F] |
| 42 | 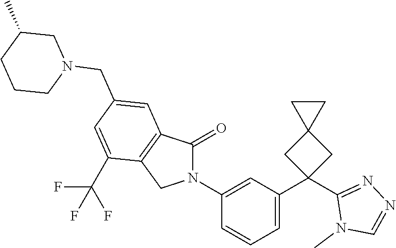 [SMILES: C[C@H]1CCCN(Cc2cc3c(c(C(F)(F)F)c2)CN(c2cccc(C4(c5nncn5C)CC5(CC5)C4)c2)C3=O)C1] |
| 43 | 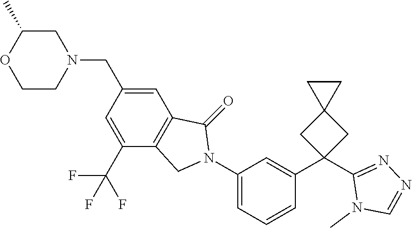 [SMILES: C[C@@H]1CN(Cc2cc3c(c(C(F)(F)F)c2)CN(c2cccc(C4(c5nncn5C)CC5(CC5)C4)c2)C3=O)CCO1] |
| 44 | 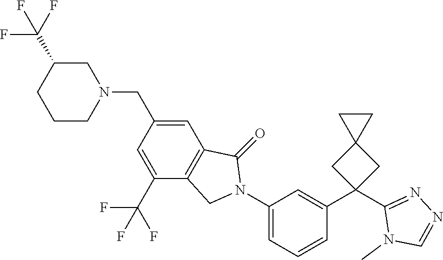 [SMILES: Cn1cnnc1C1(c2cccc(N3Cc4c(cc(CN5CCC[C@H](C(F)(F)F)C5)cc4C(F)(F)F)C3=O)c2)CC2(CC2)C1] |
| 45 | 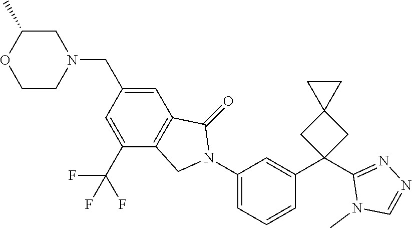 [SMILES: C[C@@H]1CN(Cc2cc3c(c(C(F)(F)F)c2)CN(c2cccc(C4(c5nncn5C)CC5(CC5)C4)c2)C3=O)CCO1] |
| 46 | 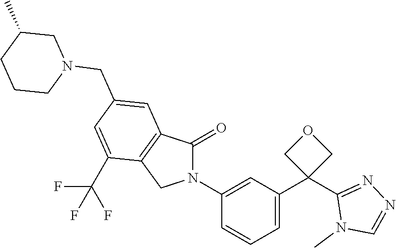 [SMILES: C[C@H]1CCCN(Cc2cc3c(c(C(F)(F)F)c2)CN(c2cccc(C4(c5nncn5C)COC4)c2)C3=O)C1] |
| 47 | 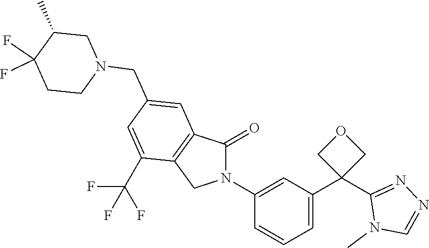 [SMILES: C[C@@H]1CN(Cc2cc3c(c(C(F)(F)F)c2)CN(c2cccc(C4(c5nncn5C)COC4)c2)C3=O)CCC1(F)F] |
| 48 | 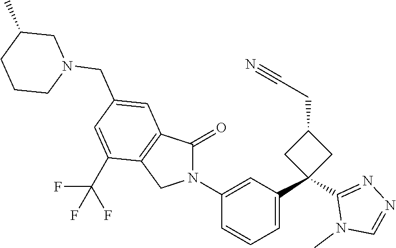 [SMILES: C[C@H]1CCCN(Cc2cc3c(c(C(F)(F)F)c2)CN(c2cccc([C@]4(c5nncn5C)C[C@@H](CC#N)C4)c2)C3=O)C1] |
| 49 | 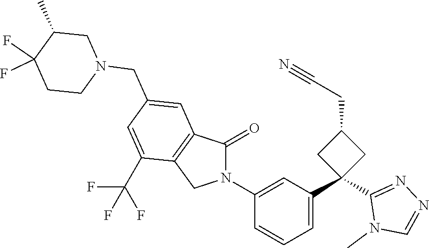 [SMILES: C[C@@H]1CN(Cc2cc3c(c(C(F)(F)F)c2)CN(c2cccc([C@]4(c5nncn5C)C[C@@H](CC#N)C4)c2)C3=O)CCC1(F)F] |
| 50 | 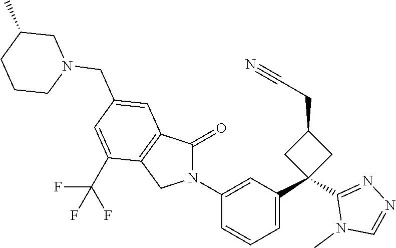 [SMILES: C[C@H]1CCCN(Cc2cc3c(c(C(F)(F)F)c2)CN(c2cccc([C@]4(c5nncn5C)C[C@H](CC#N)C4)c2)C3=O)C1] |
| 51 | 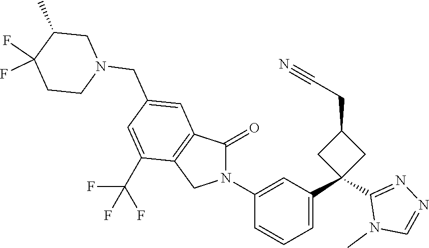 [SMILES: C[C@@H]1CN(Cc2cc3c(c(C(F)(F)F)c2)CN(c2cccc([C@]4(c5nncn5C)C[C@H](CC#N)C4)c2)C3=O)CCC1(F)F] |
| 52 | 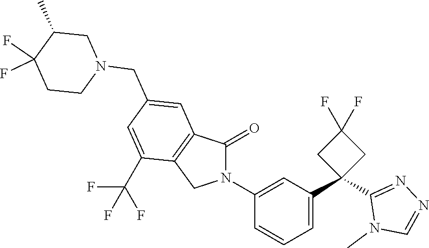 |
| 53 | 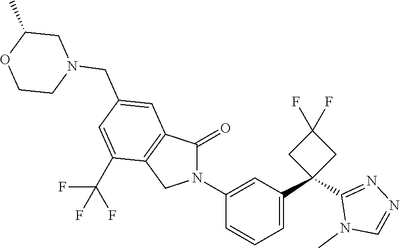 |

In certain embodiments, the compound is from Table 2, or a pharmaceutically acceptable stereoisomer, tautomer, or salt thereof.

TABLE 2
Representative Compounds of This Disclosure

| Cmpd No. | Structure |
|---|---|
| 101 | 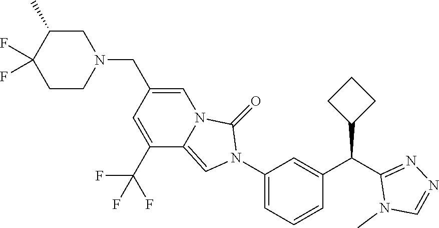 [SMILES: C[C@@H]1CN(Cc2cc(C(F)(F)F)c3cn(-c4cccc([C@H](c5nncn5C)C5CCC5)c4)c(=O)n3c2)CCC1(F)F] |
| 102 | 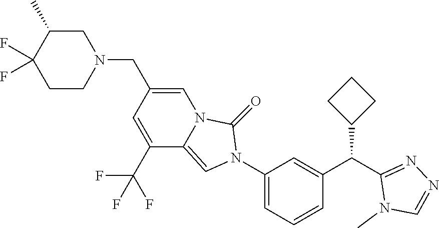 [SMILES: C[C@@H]1CN(Cc2cc(C(F)(F)F)c3cn(-c4cccc([C@@H](c5nncn5C)C5CCC5)c4)c(=O)n3c2)CCC1(F)F] |
| 103 | 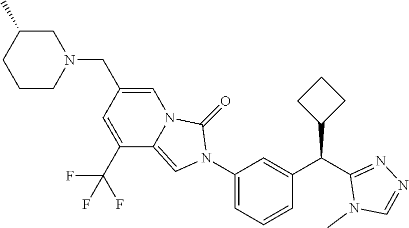 [SMILES: C[C@H]1CCCN(Cc2cc(C(F)(F)F)c3cn(-c4cccc([C@H](c5nncn5C)C5CCC5)c4)c(=O)n3c2)C1] |
| 104 | 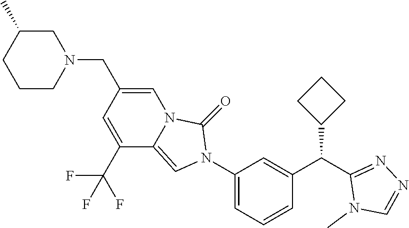 [SMILES: C[C@H]1CCCN(Cc2cc(C(F)(F)F)c3cn(-c4cccc([C@@H](c5nncn5C)C5CCC5)c4)c(=O)n3c2)C1] |
| 105 | 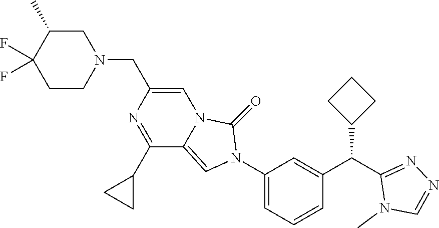 [SMILES: C[C@@H]1CN(Cc2cn3c(=O)n(-c4cccc([C@@H](c5nncn5C)C5CCC5)c4)cc3c(C3CC3)n2)CCC1(F)F] |
| 106 | 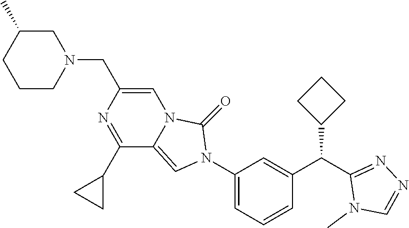 [SMILES: C[C@H]1CCCN(Cc2cn3c(=O)n(-c4cccc([C@@H](c5nncn5C)C5CCC5)c4)cc3c(C3CC3)n2)C1] |
| 107 | 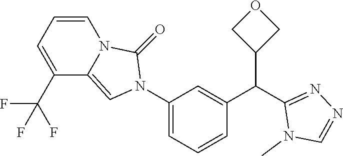 [SMILES: Cn1cnnc1C(c1cccc(-n2cc3c(C(F)(F)F)cccn3c2=O)c1)C1COC1] |
| 108 | 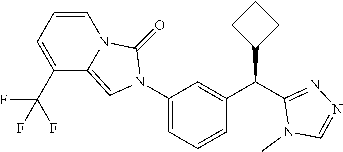 [SMILES: Cn1cnnc1[C@@H](c1cccc(-n2cc3c(C(F)(F)F)cccn3c2=O)c1)C1CCC1] |
| 109 | 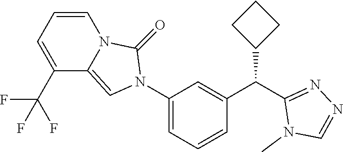 [SMILES: Cn1cnnc1[C@H](c1cccc(-n2cc3c(C(F)(F)F)cccn3c2=O)c1)C1CCC1] |
| 110 | 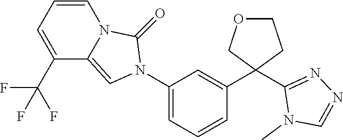 [SMILES: Cn1cnnc1C1(c2cccc(-n3cc4c(C(F)(F)F)cccn4c3=O)c2)CCOC1] |
| 111 | 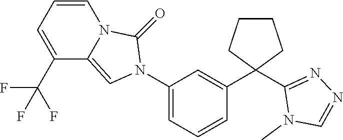 [SMILES: Cn1cnnc1C1(c2cccc(-n3cc4c(C(F)(F)F)cccn4c3=O)c2)CCCC1] |
| 114 | 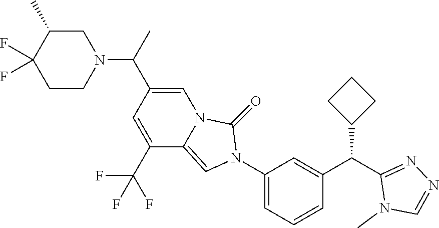 [SMILES: CC(c1cc(C(F)(F)F)c2cn(-c3cccc([C@@H](c4nncn4C)C4CCC4)c3)c(=O)n2c1)N1CCC(F)(F)[C@H](C)C1] |
| 115 | 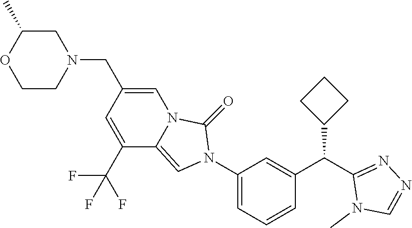 [SMILES: C[C@@H]1CN(Cc2cc(C(F)(F)F)c3cn(-c4cccc([C@@H](c5nncn5C)C5CCC5)c4)c(=O)n3c2)CCO1] |
| 116 | 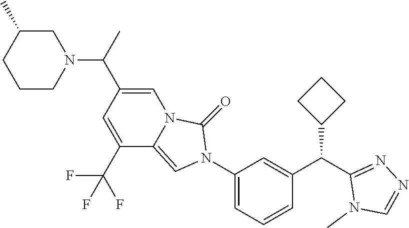 [SMILES: CC(c1cc(C(F)(F)F)c2cn(-c3cccc([C@@H](c4nncn4C)C4CCC4)c3)c(=O)n2c1)N1CCC[C@H](C)C1] |
| 117 | 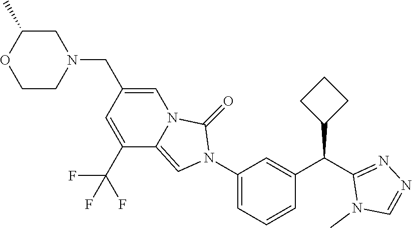 [SMILES: C[C@@H]1CN(Cc2cc(C(F)(F)F)c3cn(-c4cccc([C@H](c5nncn5C)C5CCC5)c4)c(=O)n3c2)CCO1] |
| 118 | 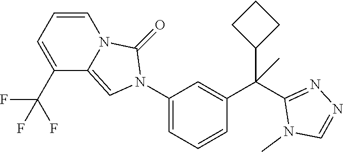 [SMILES: Cn1cnnc1C(C)(c1cccc(-n2cc3c(C(F)(F)F)cccn3c2=O)c1)C1CCC1] |
| 119 | 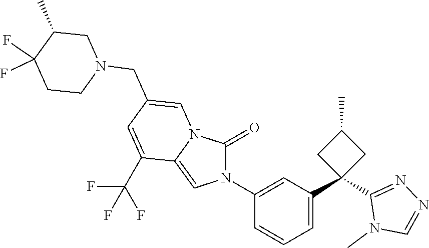 [SMILES: C[C@@H]1CN(Cc2cc(C(F)(F)F)c3cn(-c4cccc([C@]5(c6nncn6C)C[C@@H](C)C5)c4)c(=O)n3c2)CCC1(F)F] |
| 120 | 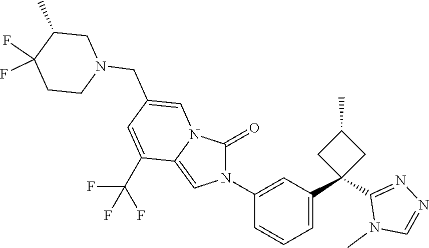 [SMILES: C[C@@H]1CN(Cc2cc(C(F)(F)F)c3cn(-c4cccc([C@]5(c6nncn6C)C[C@@H](C)C5)c4)c(=O)n3c2)CCC1(F)F] |
| 123 | 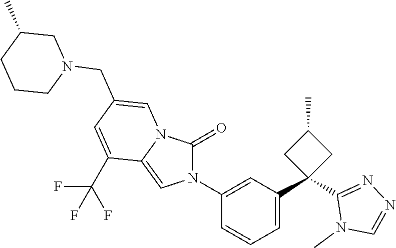 [SMILES: C[C@H]1CCCN(Cc2cc(C(F)(F)F)c3cn(-c4cccc([C@]5(c6nncn6C)C[C@@H](C)C5)c4)c(=O)n3c2)C1] |
| 124 | 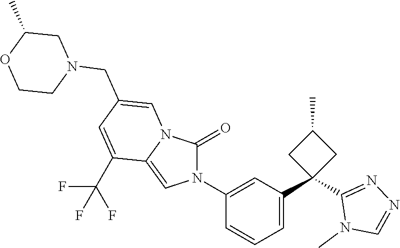 [SMILES: C[C@@H]1CN(Cc2cc(C(F)(F)F)c3cn(-c4cccc([C@]5(c6nncn6C)C[C@@H](C)C5)c4)c(=O)n3c2)CCO1] |
| 125 | 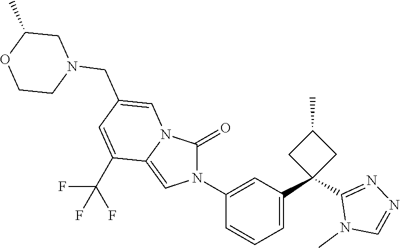 [SMILES: C[C@@H]1CN(Cc2cc(C(F)(F)F)c3cn(-c4cccc([C@]5(c6nncn6C)C[C@@H](C)C5)c4)c(=O)n3c2)CCO1] |
| 126 | 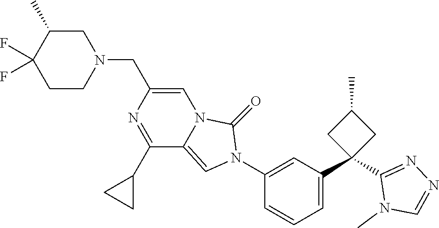 [SMILES: C[C@@H]1CN(Cc2cn3c(=O)n(-c4cccc([C@]5(c6nncn6C)C[C@@H](C)C5)c4)cc3c(C3CC3)n2)CCC1(F)F] |
| 127 | 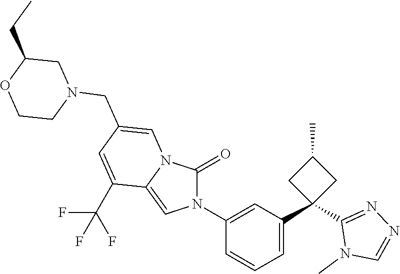 [SMILES: CC[C@H]1CN(Cc2cc(C(F)(F)F)c3cn(-c4cccc([C@]5(c6nncn6C)C[C@@H](C)C5)c4)c(=O)n3c2)CCO1] |
| 128 | 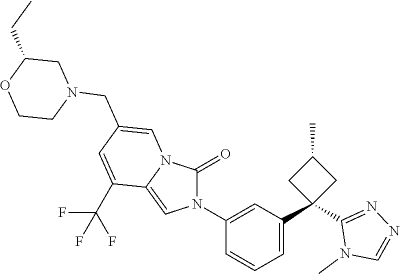 [SMILES: CC[C@@H]1CN(Cc2cc(C(F)(F)F)c3cn(-c4cccc([C@]5(c6nncn6C)C[C@@H](C)C5)c4)c(=O)n3c2)CCO1] |
| 129 | 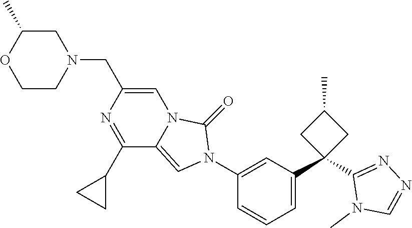 [SMILES: C[C@@H]1CN(Cc2cn3c(=O)n(-c4cccc([C@]5(c6nncn6C)C[C@@H](C)C5)c4)cc3c(C3CC3)n2)CCO1] |
| 130 | 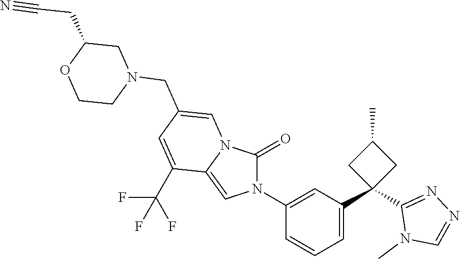 [SMILES: Cn1cnnc1[C@]1(c2cccc(-n3cc4c(C(F)(F)F)cc(CN5CCO[C@H](CC#N)C5)cn4c3=O)c2)C[C@H](C)C1] |
| 131 | 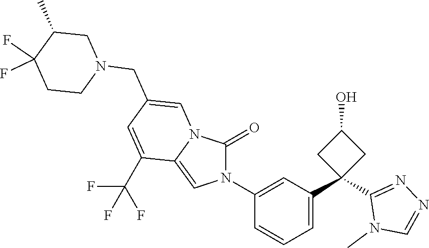 [SMILES: C[C@@H]1CN(Cc2cc(C(F)(F)F)c3cn(-c4cccc([C@]5(c6nncn6C)C[C@@H](O)C5)c4)c(=O)n3c2)CCC1(F)F] |
| 132 | 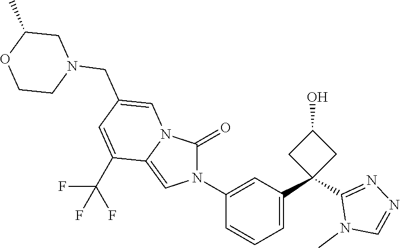 [SMILES: C[C@@H]1CN(Cc2cc(C(F)(F)F)c3cn(-c4cccc([C@]5(c6nncn6C)C[C@@H](O)C5)c4)c(=O)n3c2)CCO1] |
| 133 | 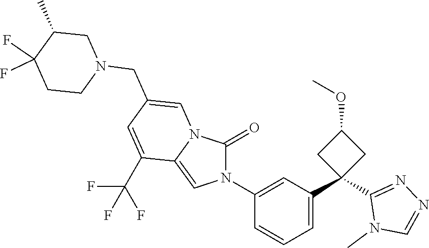 [SMILES: CO[C@H]1C[C@@](c2cccc(-n3cc4c(C(F)(F)F)cc(CN5CCC(F)(F)[C@H](C)C5)cn4c3=O)c2)(c2nncn2C)C1] |
| 134 | 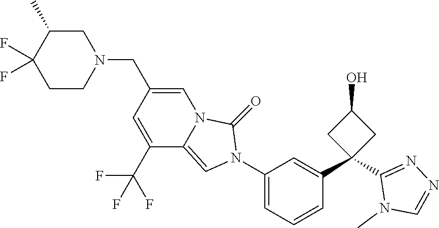 [SMILES: C[C@@H]1CN(Cc2cc(C(F)(F)F)c3cn(-c4cccc([C@]5(c6nncn6C)C[C@H](O)C5)c4)c(=O)n3c2)CCC1(F)F] |
| 135 | 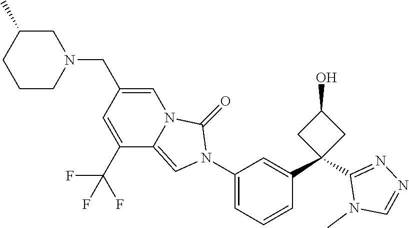 [SMILES: C[C@H]1CCCN(Cc2cc(C(F)(F)F)c3cn(-c4cccc([C@]5(c6nncn6C)C[C@H](O)C5)c4)c(=O)n3c2)C1] |
| 136 | 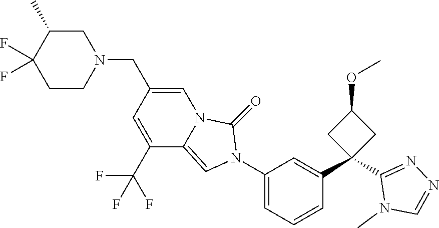 [SMILES: CO[C@H]1C[C@](c2cccc(-n3cc4c(C(F)(F)F)cc(CN5CCC(F)(F)[C@H](C)C5)cn4c3=O)c2)(c2nncn2C)C1] |
| 137 | 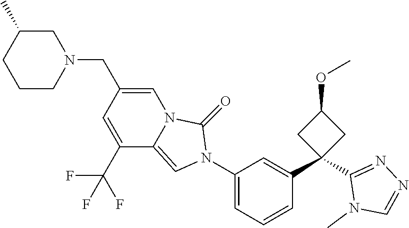 [SMILES: CO[C@H]1C[C@](c2cccc(-n3cc4c(C(F)(F)F)cc(CN5CCC[C@H](C)C5)cn4c3=O)c2)(c2nncn2C)C1] |
| 138 | 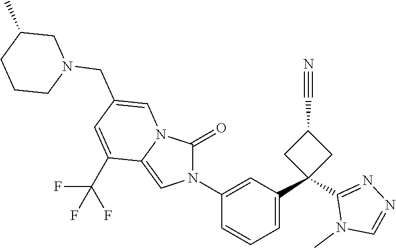 [SMILES: C[C@H]1CCCN(Cc2cc(C(F)(F)F)c3cn(-c4cccc([C@]5(c6nncn6C)C[C@@H](C#N)C5)c4)c(=O)n3c2)C1] |
| 139 | 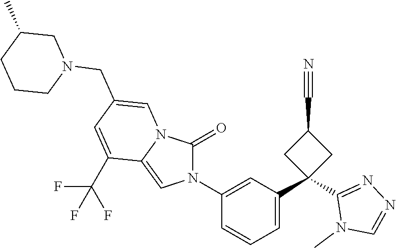 [SMILES: C[C@H]1CCCN(Cc2cc(C(F)(F)F)c3cn(-c4cccc([C@]5(c6nncn6C)C[C@H](C#N)C5)c4)c(=O)n3c2)C1] |
| 140 | 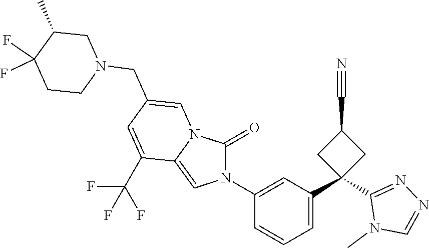 [SMILES: C[C@@H]1CN(Cc2cc(C(F)(F)F)c3cn(-c4cccc([C@]5(c6nncn6C)C[C@H](C#N)C5)c4)c(=O)n3c2)CCC1(F)F] |
| 141 | 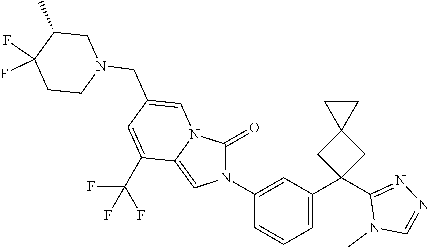 [SMILES: C[C@@H]1CN(Cc2cc(C(F)(F)F)c3cn(-c4cccc(C5(c6nncn6C)CC6(CC6)C5)c4)c(=O)n3c2)CCC1(F)F] |
| 142 | 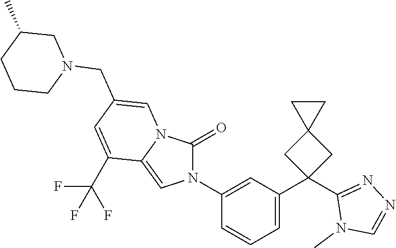 [SMILES: C[C@H]1CCCN(Cc2cc(C(F)(F)F)c3cn(-c4cccc(C5(c6nncn6C)CC6(CC6)C5)c4)c(=O)n3c2)C1] |
| 143 | 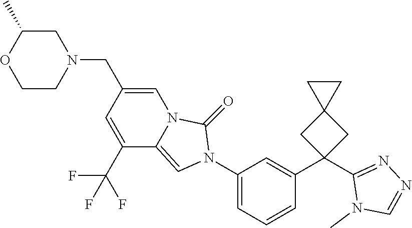 [SMILES: C[C@@H]1CN(Cc2cc(C(F)(F)F)c3cn(-c4cccc(C5(c6nncn6C)CC6(CC6)C5)c4)c(=O)n3c2)CCO1] |
| 144 | 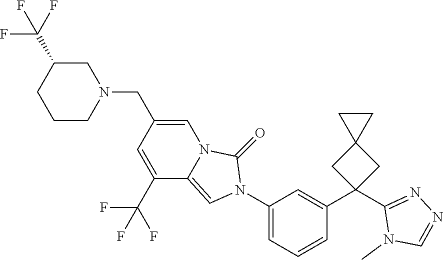 [SMILES: Cn1cnnc1C1(c2cccc(-n3cc4c(C(F)(F)F)cc(CN5CCC[C@H](C(F)(F)F)C5)cn4c3=O)c2)CC2(CC2)C1] |
| 145 | 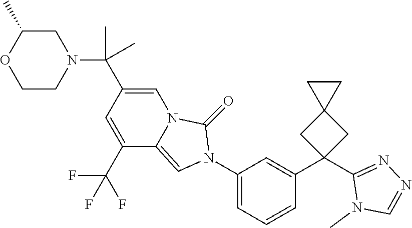 [SMILES: C[C@@H]1CN(C(C)(C)c2cc(C(F)(F)F)c3cn(-c4cccc(C5(c6nncn6C)CC6(CC6)C5)c4)c(=O)n3c2)CCO1] |
| 146 | 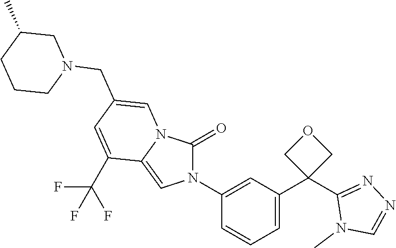 [SMILES: C[C@H]1CCCN(Cc2cc(C(F)(F)F)c3cn(-c4cccc(C5(c6nncn6C)COC5)c4)c(=O)n3c2)C1] |
| 147 | 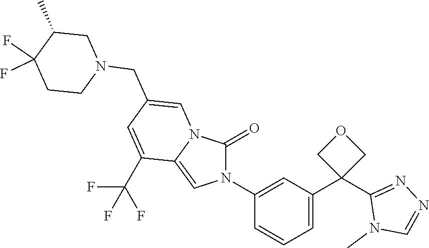 [SMILES: C[C@@H]1CN(Cc2cc(C(F)(F)F)c3cn(-c4cccc(C5(c6nncn6C)COC5)c4)c(=O)n3c2)CCC1(F)F] |
| 148 | 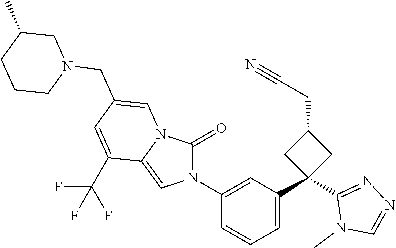 [SMILES: C[C@H]1CCCN(Cc2cc(C(F)(F)F)c3cn(-c4cccc([C@]5(c6nncn6C)C[C@@H](CC#N)C5)c4)c(=O)n3c2)C1] |
| 149 | 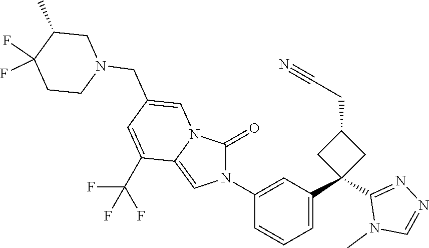 [SMILES: C[C@@H]1CN(Cc2cc(C(F)(F)F)c3cn(-c4cccc([C@]5(c6nncn6C)C[C@@H](CC#N)C5)c4)c(=O)n3c2)CCC1(F)F] |
| 150 | 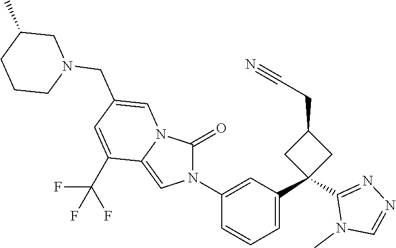 [SMILES: C[C@H]1CCCN(Cc2cc(C(F)(F)F)c3cn(-c4cccc([C@]5(c6nncn6C)C[C@H](CC#N)C5)c4)c(=O)n3c2)C1] |
| 151 | 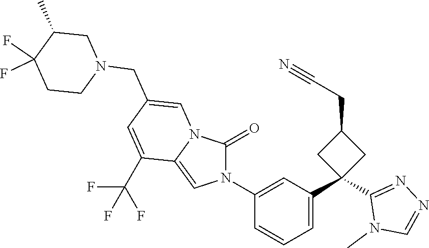 [SMILES: C[C@@H]1CN(Cc2cc(C(F)(F)F)c3cn(-c4cccc([C@]5(c6nncn6C)C[C@H](CC#N)C5)c4)c(=O)n3c2)CCC1(F)F] |
| 152 | 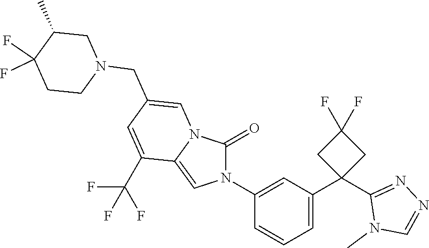 [SMILES: C[C@@H]1CN(Cc2cc(C(F)(F)F)c3cn(-c4cccc(C5(c6nncn6C)CC(F)(F)C5)c4)c(=O)n3c2)CCC1(F)F] |
| 153 | 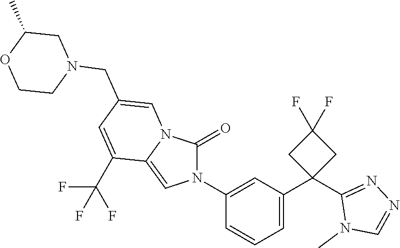 [SMILES: C[C@@H]1CN(Cc2cc(C(F)(F)F)c3cn(-c4cccc(C5(c6nncn6C)CC(F)(F)C5)c4)c(=O)n3c2)CCO1] |
| 59′ | 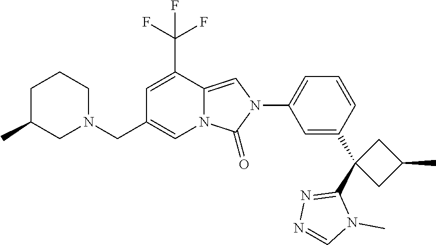 |
| 72′ | 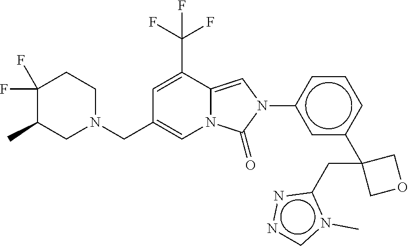 |
| 60′ | 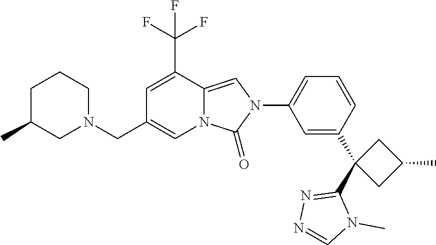 |
| 73′ | 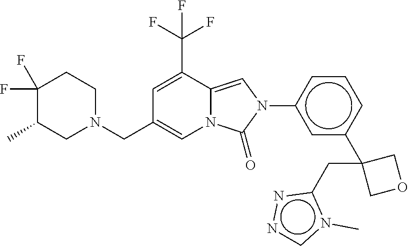 |
| 189′ | 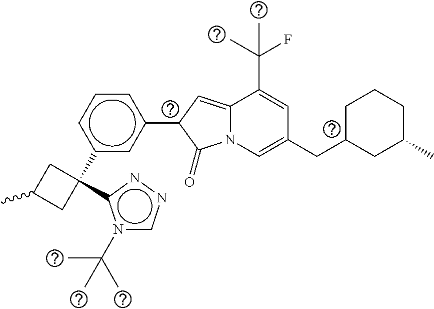 |
| 416′ | 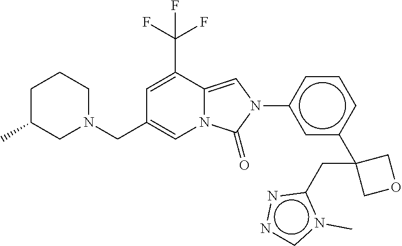 |
| 190′ | 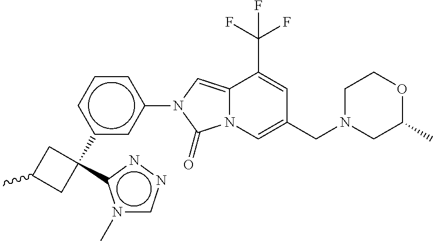 |
| 301′ | 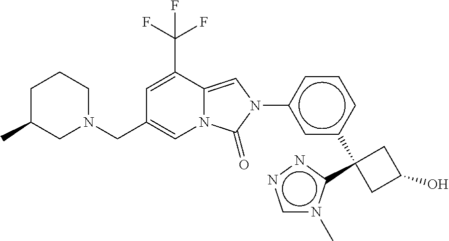 |
| 213′ | 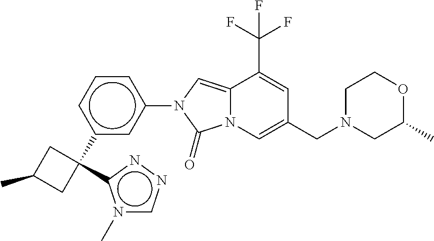 |
| 318′ | 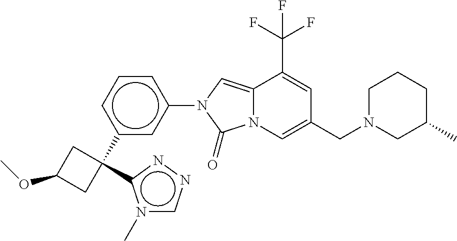 |
| 214′ | 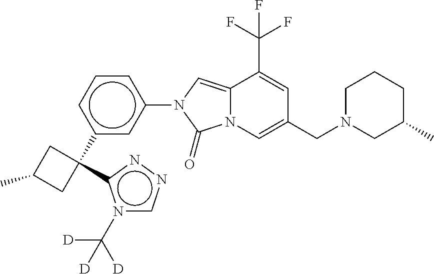 |
| 319′ | 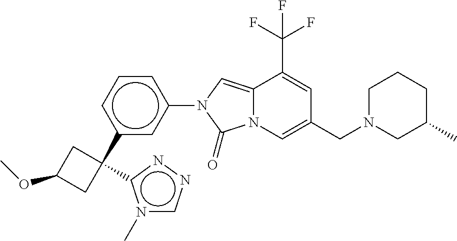 |
| 255′ | 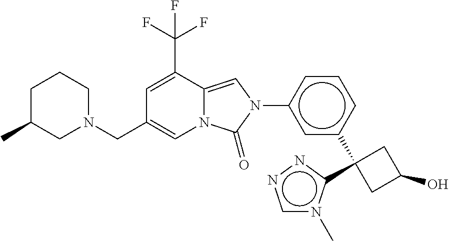 |
| 439′ | 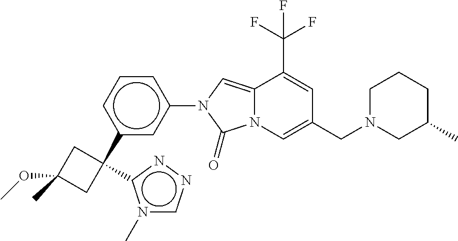 |
| 341′ | 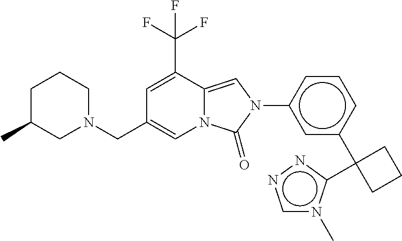 |
| 440′ | 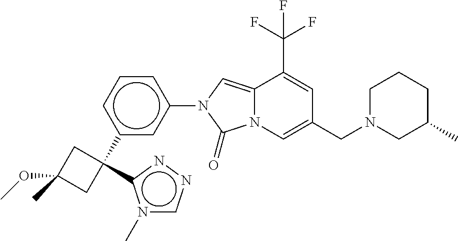 |
| 417′ | 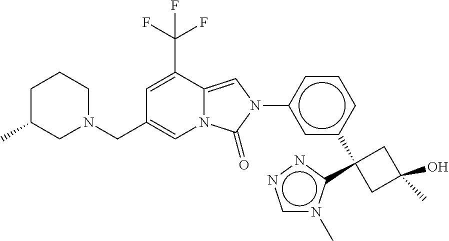 |
| 506′_P1 | 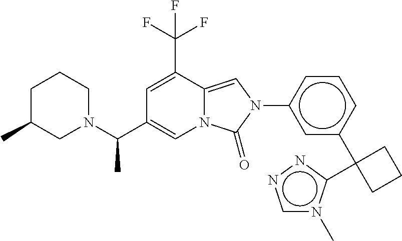 |
| 418′ | 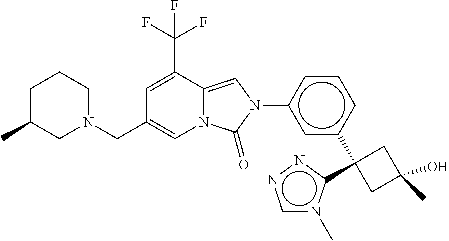 |
| 506′_P2 | 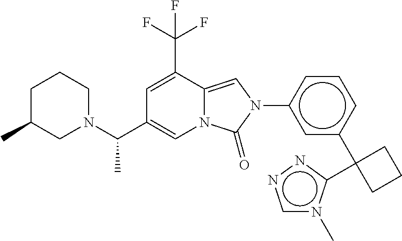 |
| 433′ | 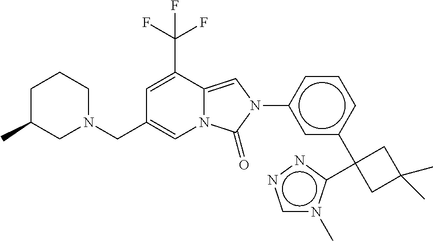 |

### 3. Combinations with a Serotonin Receptor Antagonist

Many cancer treatments including radiation are known to induce nausea and vomiting. Nausea and vomiting are protective defense responses that can be triggered by several mechanisms and are mediated by the bidirectional interaction between the gut and the brain. Because many cancer treatments target rapidly dividing cells, these treatments also tend to damage the lining of gastrointestinal tract. The resulting gut damage leads to an increase in serotonin levels as the enterochromaffin cells in the gastrointestinal tract synthesize over ninety percent of the body's serotonin. Acting as a local emetic neurotransmitter in additional to its other activities, the increased levels of serotonin stimulates the vagal afferent fibers, activates the vagal afferents in the dorsal vagal complex, and causes the cancer patient to vomit. Antiemetics can be given either prophylactically or in combination with the cancer treatment to mitigate these unwanted effects.

The Cbl inhibitors described herein are also likely to induce nausea and vomiting in patients when given doses that are likely to be therapeutically beneficial. In an unexpected finding, the Cbl inhibitors are also serotonin receptor agonists. As a consequence, Cbl inhibitor therapy can trigger the dorsal vagal complex not because of gut damage but because its presence along with the endogenous local concentration of serotonin can overwhelm the serotonin receptors in the gastrointestinal tract. Thus, treating patients with a serotonin receptor antagonist would mitigate this response but other antiemetics that act through other receptors (e.g., histamine or dopamine) would not mitigate against the response. Notably, patients can adapt to and/or tolerate Cbl inhibitors by co-administering the Cbl inhibitor and a serotonin receptor antagonist for a period of time, and then administering the Cbl inhibitor in the absence of the serotonin receptor antagonist. Optionally, in certain embodiments, a weaning step can be used wherein the dose of the serotonin receptor antagonist is reduced for a period of time. In some instances, escalating the dosage of the Cbl inhibitor with or without the serotonin receptor antagonist can also aid in patients adapting to and/or tolerating Cbl inhibitors.

In another surprising and unexpected result, the co-administration of a given dose of a Cbl inhibitor and a serotonin receptor antagonist increases the exposure of the Cbl inhibitor when compared to the administration of the same dose of Cbl inhibitor alone (i.e., without the accompanying serotonin receptor antagonist). As a consequence, when a patient is co-administered a Cbl inhibitor and a serotonin receptor antagonist in the beginning of therapy, it is as if the patient is receiving a higher loading dose of Cbl inhibitor than the actual dose of the Cbl inhibitor administered. When the patient is weaned off the serotonin receptor antagonist, the same Cbl inhibitor dose can function as a lower maintenance dose.

Provided herein are Cbl inhibitor compounds for use in combination with a serotonin receptor antagonist. Generally, the Cbl inhibitor compound and the serotonin receptor antagonist are administered according to their own doses and schedules. Thus, in certain embodiments, the Cbl inhibitor compound is administered at a dose and schedule deemed useful by the practitioner of skill. In certain embodiments, the serotonin receptor antagonist is administered at a dose and schedule deemed useful by the practitioner of skill. In particular embodiments, the serotonin receptor antagonist is administered according to its labelled instruction.

In certain embodiments, the amount of the Cbl inhibitor compound is therapeutically effective. In certain embodiments, the amount of the serotonin receptor antagonist is therapeutically effective. In certain embodiments, the amount of the Cbl inhibitor compound is therapeutically effective, and the amount of the serotonin receptor antagonist is therapeutically effective.

In certain embodiments, the Cbl inhibitor compound and the serotonin receptor antagonist are administered consecutively in either order. As used herein, the terms “consecutively,” “serially,” and “sequentially” refer to administration of a Cbl inhibitor compound after a serotonin receptor antagonist, or administration of the serotonin receptor antagonist after the Cbl inhibitor compound. For instance, consecutive administration may involve administration of the Cbl inhibitor compound in the absence of the serotonin receptor antagonist during an induction phase (primary therapy), which is followed by a post-induction treatment phase comprising administration of the serotonin receptor antagonist. The methods may further comprise a maintenance phase comprising administration of the Cbl inhibitor compound or the serotonin receptor antagonist, or both. Alternatively, consecutive administration may involve administration of the serotonin receptor antagonist in the absence of the Cbl inhibitor compound during an induction phase (primary therapy), which is followed by a post-induction treatment phase comprising administration of the Cbl inhibitor compound. The methods may further comprise a maintenance phase comprising administration of the Cbl inhibitor compound or the serotonin receptor antagonist, or both.

In certain embodiments, the Cbl inhibitor compound and the serotonin receptor antagonist are administered concurrently. As used herein, the terms “concurrently,” “simultaneously,” and “in parallel” refer to administration of a Cbl inhibitor compound and a serotonin receptor antagonist during the same doctor visit or during the same phase of treatment. For instance, both the Cbl inhibitor compound and the serotonin receptor antagonist may be administered during one or more of an induction phase, a treatment phase, and a maintenance phase. However, concurrent administration does not require that the Cbl inhibitor compound and the serotonin receptor antagonist be present together in a single formulation or pharmaceutical composition, or that the Cbl inhibitor compound and the serotonin receptor antagonist can be administered at precisely the same time.

In certain embodiments, provided herein is a method of treating cancer, the method comprising administering an effective amount of a combination provided herein to an individual to treat cancer in the individual. In certain embodiments, the individual has a cancer such as a hematologic cancer or non-hematological cancer described herein.

In certain embodiments, provided herein is a method of treating cancer responsive to inhibition of Cbl activity, such as Cbl-b activity, the method comprising administering an effective amount of a combination provided herein to an individual to treat the cancer responsive to inhibition of Cbl activity. In certain embodiments, the cancer is a hematologic cancer or non-hematological cancer such as one described herein.

The Cbl inhibitor compound or composition thereof is suitably administered to the individual at one time or over a series of treatments. In certain embodiments, the treatment includes multiple administrations of the Cbl inhibitor compound or composition, wherein the interval between administrations may vary. For example, the interval between the first administration and the second administration is about one month, and the intervals between the subsequent administrations are about three months. In certain embodiments, a Cbl inhibitor compound is administered at a flat dose. In certain embodiments, a Cbl inhibitor compound described herein is administered to an individual at a fixed dose based on the individual's weight (e.g., mg/kg).

In certain embodiments of this disclosure, the cancer is a hematologic cancer. For example, the hematologic cancer may be a lymphoma, a leukemia, or a myeloma. In other aspects of this disclosure, the cancer is a non-hematologic cancer. In particular, the non-hematologic cancer may be a carcinoma, a sarcoma, or a melanoma.

In certain embodiments, the effectiveness of the combination in the methods herein (e.g., method of modulating an immune response in an individual) can be assessed by measuring the biological activity of immune cells present in a sample (e.g., blood sample) isolated from the treated individual. For example, the ability of immune cells isolated from the individual after treatment with a combination provided herein to destroy target cells in a cytotoxicity assay may be measured to assess treatment efficacy. In certain embodiments, the biological activity of immune cells present in a sample (e.g., blood sample) can be measured by assaying expression and/or secretion of certain cytokines, such as IL-2 and IFNy.

### 4. Pharmaceutical Compositions and Methods of Administration

The Cbl inhibitor compounds and antiemetic agents provided herein can be independently formulated into pharmaceutical compositions using methods available in the art and those disclosed herein. In particular embodiments, the Cbl inhibitor compound is formulated in a pharmaceutical composition comprising the compound and one or more pharmaceutically acceptable carriers, diluents, or excipients. In certain embodiments, the antiemetic agent is formulated according to the formulations known in the art. In particular embodiments, the Cbl inhibitor compound is formulated in a pharmaceutical composition suitable for oral administration. While the Cbl inhibitor compound and the antiemetic agent are not expected to be formulated in the same composition, this embodiment is not excluded from the description herein.

The methods provided herein encompass administering pharmaceutical compositions comprising a Cbl inhibitor compound or an antiemetic agent and one or more compatible and pharmaceutically acceptable carriers. In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable, or synthetic origin, such as peanut oil, soybean oil, mineral oil, sesame oil, and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Martin, E. W., Remington's Pharmaceutical Sciences.

In clinical practice the pharmaceutical compositions for the Cbl inhibitor compounds may be administered by any route known in the art. Exemplary routes of administration include, but are not limited to, oral, intravenous, inhalation, intraarterial, intradermal, intramuscular, intraperitoneal, intravenous, nasal, parenteral, pulmonary, and subcutaneous routes. In certain embodiments, a pharmaceutical composition provided herein is administered parenterally.

The Cbl inhibitor compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing, and stabilizing agents. Sterilization can be carried out in several ways, for example using a bacteriological filter, by radiation or by heating. Parenteral compositions can also be prepared in the form of sterile solid compositions which can be dissolved at the time of use in sterile water or any other injectable sterile medium.

In general, the need for an antiemetic agent in combination with a Cbl inhibitor is when the Cbl inhibitor compound is administered orally. When the route of administration is not oral, then the need for an antiemetic agent may be mitigated. Should a patient require an antiemetic agent because the patient is experiencing symptoms, then any route known in the art may be used.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic agents.

The pharmaceutical composition may comprise one or more pharmaceutical excipients. Any suitable pharmaceutical excipient may be used, and one of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Whether a particular excipient is suitable for incorporation into a pharmaceutical composition or dosage form depends on a variety of factors well known in the art including, but not limited to, the way in which the dosage form will be administered to a subject and the specific dosage form. The composition or single unit dosage form, if desired, can also contain minor amounts of wetting or emulsifying agents, or pH buffering agents. Accordingly, the pharmaceutical excipients provided herein are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In certain embodiments, the pharmaceutical composition comprises an anti-foaming agent. Any suitable anti-foaming agent may be used. In certain embodiments, the anti-foaming agent is selected from an alcohol, an ether, an oil, a wax, a silicone, a surfactant, and combinations thereof In certain embodiments, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof

In certain embodiments, the pharmaceutical composition comprises a co-solvent. Illustrative examples of co-solvents include ethanol, poly(ethylene) glycol, butylene glycol, dimethylacetamide, glycerin, and propylene glycol.

In certain embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In certain embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In certain embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In certain embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press.

In certain embodiments, the pharmaceutical composition comprises a solvent. In certain embodiments, the solvent is saline solution, such as a sterile isotonic saline solution or dextrose solution. In certain embodiments, the solvent is water for injection.

In certain embodiments, the pharmaceutical compositions are in a particulate form, such as a microparticle or a nanoparticle. Microparticles and nanoparticles may be formed from any suitable material, such as a polymer or a lipid. In certain embodiments, the microparticles or nanoparticles are micelles, liposomes, or polymersomes.

Further provided herein are anhydrous pharmaceutical compositions and dosage forms comprising therapeutic agent, since, in certain embodiments, water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Also provided are pharmaceutical compositions and dosage forms that comprise one or more excipients that reduce the rate by which the dosage form will decompose. Such excipients, which are referred to herein as “stabilizers,” include, but are not limited to, antioxidants such as ascorbic acid, pH buffers, or salt buffers.

In human therapeutics, the doctor will determine the posology which he considers most appropriate according to a preventive or curative treatment and according to the age, weight, condition, and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies.

The amounts of the Cbl inhibitor compound or composition and the antiemetic agent or composition which will be effective in the prevention or treatment of a disorder or one or more symptoms thereof will vary with the nature and severity of the disease or condition, and the route by which the agents are administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, the dosage of the Cbl inhibitor compound is 0.1 mg to 1000 mg, 0.1 mg to 900 mg, 0.1 mg to 800 mg, 0.1 mg to 750 mg, 0.1 mg to 700 mg, 0.1 mg to 600 mg, mg to 500 mg, 0.1 mg to 400 mg, 0.1 mg to 300 mg, 0.1 mg to 250 mg, 0.1 mg to 200 mg, 0.1 mg to 200 mg, 0.1 mg to 100 mg, 0.1 mg to 50 mg, 1 mg to 50 mg, 10 mg to 50 mg, 15 mg to 30 mg, 20 mg to 30 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 mg to 2.5 mg, 0.25 mg to 20 mg, 0.25 mg to 15 mg, 0.25 mg to 12 mg, 0.25 mg to 10 mg, 0.25 mg to 7.5 mg, 0.25 mg to 5 mg, 0.25 mg to 2.5 mg, 0.5 mg to 20 mg, 0.5 to 15 mg, 0.5 mg to 12 mg, 0.5 mg to 10 mg, 0.5 mg to 7.5 mg, 0.5 mg to 5 mg, 0.5 mg to 2.5 mg, 1 mg to 30 mg, 1 mg to 25 mg, 5 mg to 50 mg, 10 mg to 50 mg, 10 mg to 40 mg, 10 mg to mg, 15 mg to 50 mg, 15 mg to 40 mg, 15 mg to 35 mg, 15 mg to 25 mg, 20 mg to 50 mg, 20 mg to 40 mg, or 25 mg to 35 mg. In certain embodiments, the dosage of the Cbl-b inhibitor compound is 5 mg, 10 mg, 15 mg, 20 mg, 25 mg, 30 mg, 35 mg, 40 mg, 45 mg, 50 mg, 55 mg, 60 mg, 65 mg, mg, or 75 mg. In certain embodiments the dosage of the Cbl-b inhibitor compound is less than 100 mg, less than 90 mg, less than 80 mg, less than 70 mg, less than 60 mg, less than 50 mg, less than 40 mg, or less than 30 mg.

In certain embodiments, the dosage of the Cbl inhibitor compound is 15 mg once daily, mg twice daily, 25 mg once daily, 25 mg twice daily, 50 mg once daily, or 50 mg twice daily. In certain embodiments, the dosage of the Cbl inhibitor compound is 15 mg once daily. In certain embodiments, the dosage of the Cbl inhibitor compound is 15 mg twice daily. In certain embodiments, the dosage of the Cbl inhibitor compound is 25 mg once daily. In certain embodiments, the dosage of the Cbl inhibitor compound is 25 mg twice daily. In certain embodiments, the dosage of the Cbl inhibitor compound is 50 mg once daily. In certain embodiments, the Cbl inhibitor compound is 50 mg twice daily.

In certain embodiments, the dosage of the Cbl inhibitor compound can be increased over time. In certain embodiments, the dosage of the Cbl inhibitor can be increased from 15 mg to mg or from 15 mg to 25 mg to 50 mg over several daily doses. In certain embodiments, the dosage of the Cbl inhibitor is 15 mg daily for one week, 20 mg daily for one week, then 25 mg for three weeks. Depending on the patient's tolerance, the patient then may stay on 25 mg daily or escalated to 30 mg daily for three weeks and optionally escalated to 35 mg daily thereafter. In certain embodiments, the dosage of the Cbl inhibitor is 15 mg daily for one week, 25 mg daily for one week, then 30 mg daily thereafter. In certain embodiments, the dosage of the Cbl inhibitor can be increased from 15 mg daily to 35 mg daily over several weeks increasing the dose by 5 mg every week (15 mg daily for one week, 20 mg daily for the second week, 25 mg daily for the third week, 30 mg daily for the fourth week then 35 mg daily thereafter). In certain embodiments, the dosage of the Cbl inhibitor can be increased from 15 mg to 25 mg to 50 mg over several daily doses. In certain embodiments, the dose can be increased from one dose to a higher dose over a period of a week, two weeks, three weeks, or one month.

In certain embodiments, the dosage of the Cbl inhibitor compound provided herein, based on weight of the compound, administered to prevent, treat, manage, or ameliorate a disorder, or one or more symptoms thereof in a subject is 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 10 mg/kg, or 15 mg/kg or more of a subject's body weight.

In certain embodiments, the dosage of the serotonin receptor antagonist is according to the product label or other instruction. In certain embodiments, the dosage of the antiemetic agent based on weight of the antiemetic agent, administered to prevent, treat, manage, or ameliorate nausea, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the serotonin receptor antagonist is 0.1 mg to 1000 mg, 0.1 mg to 900 mg, 0.1 mg to 800 mg, 0.1 mg to 750 mg, 0.1 mg to 700 mg, 0.1 mg to 600 mg, 0.1 mg to 500 mg, 0.1 mg to 400 mg, 0.1 mg to 300 mg, 0.1 mg to 250 mg, 0.1 mg to 200 mg, 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 mg to 2.5 mg, 0.25 mg to 20 mg, 0.25 mg to 15 mg, 0.25 mg to 12 mg, 0.25 mg to 10 mg, 0.25 mg to 7.5 mg, 0.25 mg to 5 mg, 0.25 mg to 2.5 mg, 0.5 mg to 20 mg, 0.5 mg to 15 mg, 0.5 mg to 12 mg, 0.5 mg to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg. In certain embodiments, the dosage of the serotonin receptor antagonist is 4 mg, 8 mg, 16 mg, or 24 mg. In certain embodiments, the dosage of the serotonin receptor antagonist is 0.5 mg, 1 mg, 1. 5mg, or 2 mg. In certain embodiments, the dosage of the serotonin receptor antagonist is 90 mg, 95 mg, 100 mg, or 110 mg.

In an example, the serotonin receptor antagonist can be ondansetron that is administered at 8 mg, 16 mg/dose, or 24 mg/dose as a single oral dose. In an example, the antiemetic can be granisetron that is administered at 1 mg or 2 mg as a single oral dose. In an example, the serotonin receptor antagonist can be palonosetron administered at 0.25 mg, 0.5 mg, or 0.75 mg as a single oral dose. In an example, the antiemetic can be dolasetron that is administered at 100 mg or 200 mg as a single oral dose.

The dose of the serotonin receptor antagonist can be administered according to a suitable schedule, for example, once, two times, three times, or four times weekly. It may be necessary to use dosages of the agents outside the ranges disclosed herein in some cases, as will be apparent to those of ordinary skill in the art. Furthermore, it is noted that the clinician or treating physician will know how and when to interrupt, adjust, or terminate therapy in conjunction with subject response. In an example, the serotonin receptor antagonist administration is stopped after 1 week, after 2 weeks, after 3 weeks, or after 4 weeks. In an example, the dosage of the serotonin receptor antagonist can be periodically reduced overtime to gradually wean the patient off the serotonin receptor while maintaining the administration of the Cbl inhibitor compound. In certain embodiments, the serotonin receptor antagonist administration is reduced after 1 week, after 2 weeks, after 3 weeks, or after 4 weeks.

In certain embodiments, the serotonin receptor antagonist is ondanesetron. A dose of 8 mg of ondanesetron can be administered at least 30 minutes or an hour before the Cbl inhibitor compound administration. In certain embodiments, a dose of 16 mg of ondanesetron can be administered before the Cbl inhibitor compound administration. In certain embodiments, a dose of 24 mg of ondanesetron can be administered before the Cbl inhibitor compound administration.

In certain embodiments, the serotonin receptor antagonist is granisetron. A dose of 1 mg of granisetron can be administered one hour before the Cbl inhibitor compound administration. In certain embodiments, a dose of 2 mg of granisetron can be administered before the Cbl inhibitor compound administration.

In certain embodiments, the serotonin receptor antagonist is dolasetron. A dose of 100 mg of dolasetron can be administered at least one hour before the Cbl inhibitor compound administration. In certain embodiments, a dose of 200 mg of dolesetron can be administered before the Cbl inhibitor compound administration.

In certain embodiments, the serotonin receptor antagonist is palonosetron. A dose of 0.25 mg of palonosetron can be administered at least 30 minutes before the Cbl inhibitor compound administration. In certain embodiments, a dose of 0.5 mg of palonosetron can be administered at least 30 minutes before the Cbl inhibitor compound administration. In certain embodiments, a dose of 0.75 mg of palonosetron can be administered at least 30 minutes before the Cbl inhibitor compound administration.

Different therapeutically effective amounts may be applicable for different diseases and conditions, as will be readily known by those of ordinary skill in the art. Similarly, amounts sufficient to prevent, manage, treat, or ameliorate such disorders, but insufficient to cause, or sufficient to reduce, adverse effects associated with the agents provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an agent provided herein followed by one or more maintenance doses.

In certain embodiments, a dose of an agent provided herein can be administered to achieve a steady-state concentration of the Cbl inhibitor in blood or serum of the subject. The steady-state concentration can be determined by measurement according to techniques available to those of skill or can be based on the physical characteristics of the subject such as height, weight, and age.

### 5. Therapeutic Applications

The combinations provided herein may be useful for the treatment of any disease or condition involving abnormal cell growth or proliferation. In certain embodiments, the disease or condition is a disease or condition that can benefit from treatment with a Cbl inhibitor compound or an antiemetic agent, or both. In certain embodiments, the disease or condition is a cancer and side effects of cancer and/or cancer treatment. In certain embodiments, the disease or condition is a solid tumor. In certain embodiments, the disease or condition is a hematological cancer.

Any suitable cancer may be treated with combinations provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer (including triple-negative breast cancer, or TNBC), bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fallopian tube carcinoma, fibrous histiocytoma, Ewing sarcoma, eye cancer, germ cell tumor, gallbladder cancer, gastric cancer, gastric/gastroesophageal junction (GEJ) cancer, gastrointestinal carcinoid tumor, gastrointestinal stromal tumor, gestational trophoblastic disease, glioma, head and neck cancer, hepatocellular cancer, histiocytosis, Hodgkin lymphoma, diffuse large B cell lymphoma (DLBCL) including Richter transformation (RT), hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, metastatic melanoma, Merkel cell carcinoma, mesothelioma, malignant pleural mesothelioma (VIPM), metastatic squamous neck cancer with occult primary, squamous cell carcinoma of the head and neck (HNSCC), midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer (NSCLC), oropharyngeal cancer, osteosarcoma, ovarian cancer, platinum-resistant epithelial ovarian cancer (EOC), pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, primary peritoneal carcinoma, prostate cancer, metastatic castration-resistant prostate cancer (mCRPC), rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, urothelial cancer, muscle-invasive urothelial cancer, and Wilms tumor.

In certain embodiments, the disease to be treated with the combinations provided herein is gastric cancer, colorectal cancer, renal cell carcinoma, cervical cancer, non-small cell lung carcinoma, ovarian cancer, uterine cancer, fallopian tube carcinoma, primary peritoneal carcinoma, uterine corpus carcinoma, endometrial carcinoma, prostate cancer, breast cancer, head and neck cancer, brain carcinoma, liver cancer, pancreatic cancer, mesothelioma, and/or a cancer of epithelial origin. In particular embodiments, the disease is colorectal cancer. In certain embodiments, the disease is ovarian cancer. In certain embodiments, the disease is breast cancer. In certain embodiments, the disease is triple-negative breast cancer (TNBC). In certain embodiments, the disease is lung cancer. In certain embodiments, the disease is non-small cell lung cancer (NSCLC). In certain embodiments, the disease is head and neck cancer. In certain embodiments, the disease is renal cell carcinoma. In certain embodiments, the disease is brain carcinoma. In certain embodiments, the disease is endometrial cancer.

### 6. Kits

In certain embodiments, a compound or combination provided herein is provided in the form of a kit, i.e., a packaged combination of reagents in predetermined amounts with instructions for performing a procedure. In other embodiments, the procedure is a therapeutic procedure. In certain embodiments, the kit comprises a Cbl inhibitor compound, or a composition thereof, and instructions for use in combination with an antiemetic agent. In certain embodiments, the kit comprises an antiemetic agent, or a composition thereof, and instructions for use in combination with a Cbl inhibitor compound. In certain embodiments, the kit comprises a Cbl inhibitor compound, or a composition thereof, and an antiemetic agent, or composition thereof.

### EXAMPLES

Administration of compound 23 leads to increased levels of 5-HT in the local gut, which leads to vagal response via gut-brain vagal afferents. Specifically, compound 23 at doses of 25 mg and 50 mg single daily dose administed fasting (only with up to 8 oz, 320 mL) induced within 2 hours signficant events of nausea, vomiting, synocope, tachycardia, hypotension, and bradycardia.

Prior to administration of compound 23, the antiemetic was administered as outlined below.

Example 1: ondansetron via IV: 8 mg or 0.15 mg/kg as single dose; maximum dose of 16 mg/dose. Example 1B: ondansetron via oral: 8 mg up to twice daily. In this example, the patient was weaned off ondansetron via oral after two weeks. Thereafter, compound 23 was admistered daily without the need for ondanesetron.

Example 2: granisetron via IV: 1 mg or 10 mcg/kg as a single dose. Example 2B granisetron via oral: 2 mg as a single dose. In this example, the patient was weaned off oral granisetron after four weeks. Thereafter, compound 23 was admistered daily without the need for granisetron. Example 2C: granisetron via SUBQ: 10 mg as single dose. Example 2D: granisetron via transdermal: apply one patch.

Example 3: palonosetron via IV: 0.25 mg on day 1 prior to chemotherapy. Example 3B: palonosetron via oral: 0.5 mg on day 1 prior to chemotherapy.

Example 4: dolasetron via oral: 100 mg once daily.

Example 5: metoclopramide, a dopamine antagonist, via oral: 10 mg 30 minutes prior to the adminstration of compound 23. The administration of a dopamine antagonist did not control the patient's nausea and vomiting sympotms.

Example 6: diphenhydramine, a histamine antagonist, via oral or parental: 25 mg or 50 mg 30 minutes prior to the adminstration of compound 23. The administration of a histamine antagonist did not control the patient's nausea and vomiting sympotms.

In each example, local concentration of compound 23 was 25 mg/320 cc >2004.

The nausea and vomiting symptoms were mitigated when a single oral (versus other routes of adminstration such as intravenous) dose of 5-HT3 antagonist was administered 30-60 minutes prior to the compound 23 dosing. Both ondansetron and granisetron reduced emetic symptoms. The adverse events (e.g., symptoms include nausea, vomiting, headache, hypotension, tachycardia, bradycardia, among others) were resolved within 24 hours prior to the next dose. The nausea and vomiting were controlled by serotonin (5-HT3) receptor blockers, ondansetron and granisetron.

The seratonin receptor antagonist can be weaned off after 2-4 weeks as tolerated. Optionally, the dose of the antiemetic can be reduced by 50% for one week, then discontinuing.

Example 7: Pharmacokinetic Analysis

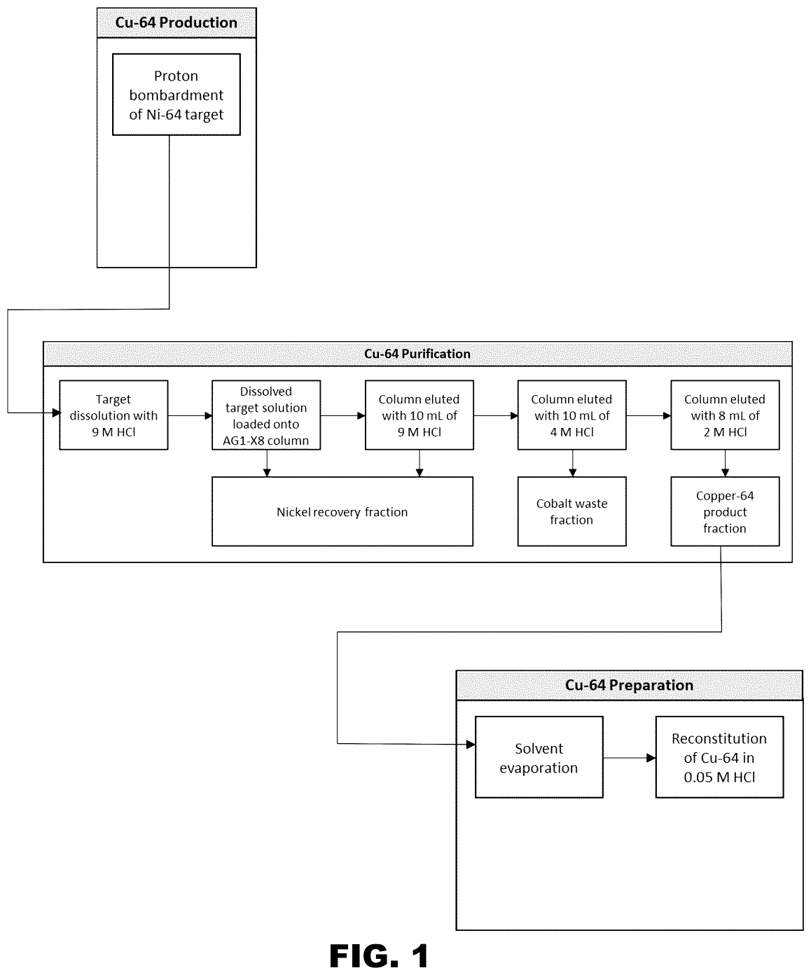

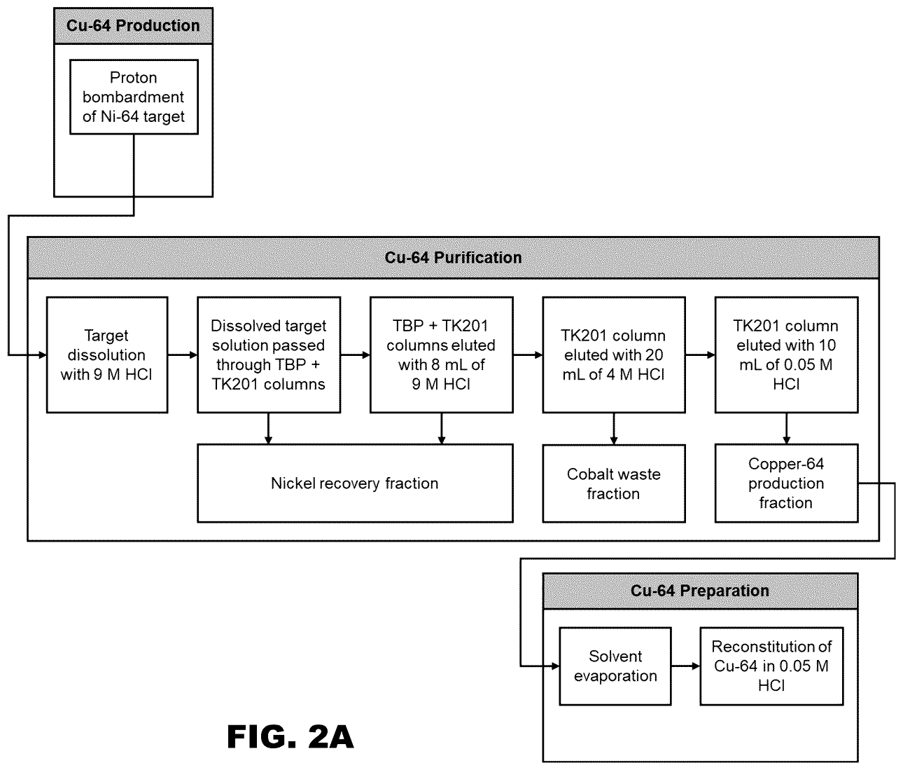

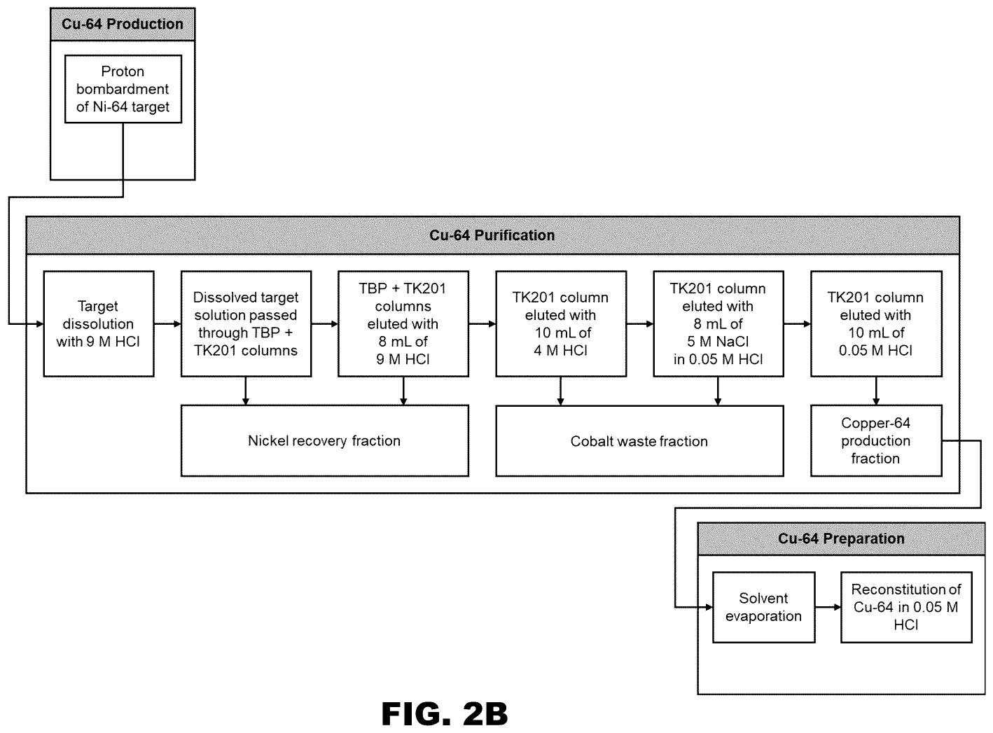

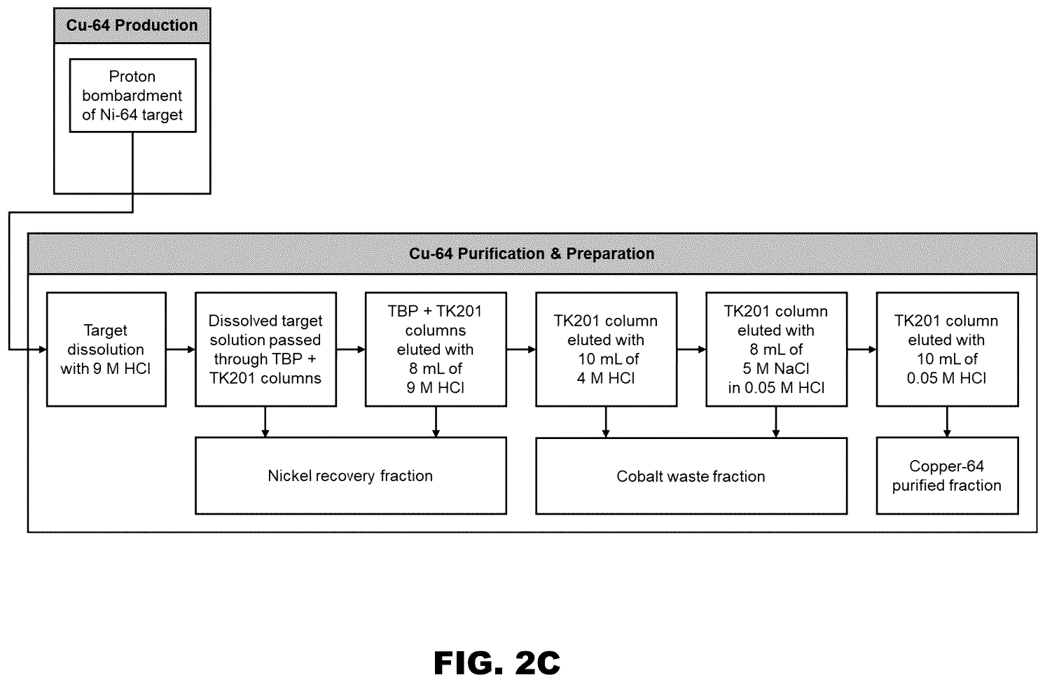

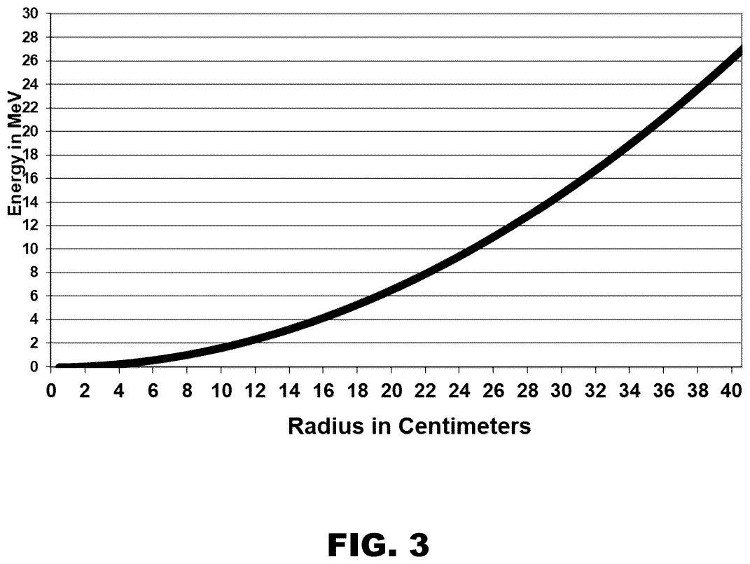

Patients were administered doses of compound 23 ranging from 5 to 50 mg once daily. Plasma concentrations of compound 23 were quantified using acetonitrile precipitation and LC-MS/MS. The peak area of the product ion of the analyte was measured against the peak area of the product ion of the stable label internal standard. A calibration curve spanning the curve range and containing at least six conentrations in duplicate was used to quantify the analyte concentration. The retention times of the analyte and internal standard are approximately 1.5 minutes. The resulting PK data are shown in FIG. 1. PK analyses were donucted using non-compartmental analyses to obtain PK parameters. PK analysis of compound 23 suggests a dose-proportional increase in exposures with no accumulation upon repeat daily dosing. Patients were administered doses of compound 23 ranging from 5 to 50 mg once daily. Plasma concentrations of compound 23 were quantified using acetonitrile precipitation and LC-MS/MS. The peak area of the product ion of the analyte was measured against the peak area of the product ion of the stable label internal standard. A calibration curve spanning the curve range and containing at least six conentrations in duplicate was used to quantify the analyte concentration. The retention times of the analyte and internal standard are approximately 1.5 minutes. The resulting PK data are shown in FIG. 1. PK analyses were donucted using non-compartmental analyses to obtain PK parameters. PK analysis of compound 23 suggests a dose-proportional increase in exposures with no accumulation upon repeat daily dosing. PK data are provided below (Cₘₐₓ and AUC₀₋ₗₐₛₜ are presented as geometric mean (geometric %C V); Tmax is presented as median (range); t_{1/2} is presented as mean (% CV).

Cycle 1 Day 1

| | Cₘₐₓ | AUC₀₋ₗₐₛₜ | Tₘₐₓ | t_{1/2} |
|---|---|---|---|---|
| Dose | (ng/mL) | (h*ng/ml) | (h) | (h) |
| 5 mg (n = 1) | 4.35 | 26.2 | 2.0 | 7.72 |
| 15 mg (n = 9) | 16.2 | 129 | 2.0 | 7.14 |
| | (38.5) | (33.4) | (1.5-6.0) | (19.8) |
| 25 mg (n = 6) | 30.1 | 201 | 1.5 | 6.82 |
| | (109) | (103) | (1.0-3.0) | (27.5) |
| 50 mg (n = 2) | 79.2 | 502 | 2.5 | 5.88 |
| | (134) | (113) | (2.0-3.0) | (7.7) |

### 8. Equivalents

The disclosure set forth above may encompass multiple distinct embodiments with independent utility. Although each of these embodiments has been disclosed in a certain form(s), the specific embodiments thereof as disclosed and illustrated herein are not to be considered in a limiting sense, because numerous variations are possible. The subject matter of this disclosure includes all novel and nonobvious combinations and subcombinations of the various elements, features, functions, and/or properties disclosed herein. The following claims particularly point out certain combinations and subcombinations regarded as novel and nonobvious. Embodiments in other combinations and subcombinations of features, functions, elements, and/or properties may be claimed in this application, in applications claiming priority from this application, or in related applications. Such claims, whether directed to a different embodiment or to the same embodiment, and whether broader, narrower, equal, or different in scope in comparison to the original claims, also are regarded as included within the subject matter of the present disclosure.

One or more features from any embodiments described herein or in the figures may be combined with one or more features of any other embodiments described herein or in the figures without departing from the scope of this disclosure.

All publications, patents, and patent applications cited in this specification are herein incorporated by reference as if each individual publication, patent, or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed is:

1. A method of treating or preventing a disease or condition in a subject in need thereof, comprising administering to the subject:
(a) an effective amount of Cbl inhibitor compound; and
(b) a serotonin receptor antagonist.

2. The method of claim 1, wherein the duration of the administration of the Cbl inhibitor compound is greater than the duration of the administration of the serotonin receptor antagonist.

3. The method of claim 2, wherein the dose of the serotonin receptor antagonist is periodically reduced during at least a portion of the duration of the administration of the Cbl inhibitor compound.

4. The method of any of claims 2-3, wherein the dose of the serotonin receptor antagonist is reduced after 2-4 weeks. The method of any of claims 2-4, wherein the dose of the serotonin receptor antagonist is eliminated after 2-4 weeks.

6. The method of any of the previous claims, wherein the serotonin receptor antagonist is ondansetron, optionally wherein the dose of the ondansetron is 8 mg, 16 mg or 24 mg.

7. The method of any of the previous claims, wherein the serotonin receptor antagonist is granisetron, optionally wherein the dose of the granisetron is 1 mg or 2 mg.

8. The method of any of the previous claims, wherein the serotonin receptor antagonist is palonosetron, optionally wherein the dose of the palonosetron is 0.25 mg, 0.5 mg or mg.

9. The method of any of the previous claims, wherein the serotonin receptor antagonist is dolasetron, optionally wherein the dose of the dolasetron is 100 mg or 200 mg.

10. A method of treating or preventing nausea or vomiting, or both, in a patient undergoing Cbl treatment comprising administering to the subject:
an effective amount of a serotonin receptor antagonist.

11. The method of claim 10, wherein the serotonin receptor antagonist is administered at least 30 minutes prior to administration of the Cbl inhibitor.

12. The method of claim 10, wherein a duration of the administration of the Cbl inhibitor compound is greater than a duration of the administration of the serotonin receptor antagonist.

13. The method of claim 10, wherein the dose of the serotonin receptor antagonist is periodically reduced during at least a portion of the duration of the administration of the Cbl inhibitor compound.

14. The method of any of claims 10-13, wherein the serotonin receptor antagonist is selected from the group consisting of ondansetron, granisetron, palonosetron, dolasetron, and combinations thereof.

15. The method of any of claims 10-14, wherein the serotonin receptor antagonist is selected from the group consisting of ondansetron, granisetron, and combinations thereof.

16. The method of any of the previous claims, wherein the Cbl inhibitor is a Cbl-b inhibitor.

17. The method of any of the previous claims, wherein the Cbl inhibitor is a c-Cbl inhibitor.

18. The method of any of the previous claims, wherein the Cbl inhibitor is a Cbl-b inhibitor and a c-Cbl inhibitor.

19. The method of any of the previous claims, wherein the Cbl-b inhibitor compound is according to Formula (I), or a pharmaceutically acceptable stereoisomer, tautomer, salt, or solvate thereof 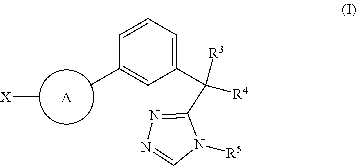
or a tautomer thereof, or a pharmaceutically acceptable salt thereof, wherein 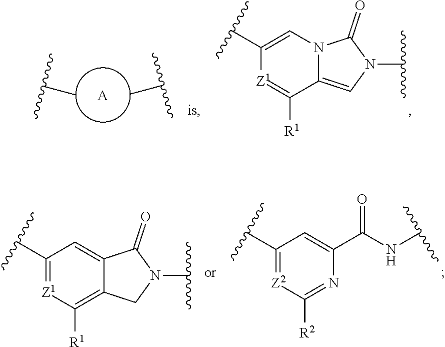
Z¹ is CH or nitrogen;
Z² is CH or nitrogen;
R¹ is —CF₃ or cyclopropyl;
R² is —CF₃ or cyclopropyl;
R³ is hydrogen, C₁-C₂ alkyl, or C₁-C₂ haloalkyl;
R⁴ is hydrogen, C₁-C₆ alkyl, C₁-C₆ haloalkyl, 4- to 8-membered heterocyclyl, or C₃-C₆ cycloalkyl, wherein the heterocyclyl or cycloalkyl groups are optionally substituted by one to five R⁶ groups;
or R³ and R⁴ are taken together with the carbon atom to which they are attached to form a C₃-C₅ cycloalkyl or 4- to 6-membered heterocyclyl, each of which is optionally substituted by one to five R⁶ groups;
R⁵ is hydrogen, C₁-C₆ alkyl, C₁-C₆ haloalkyl, or C₃-C₆ cycloalkyl;
each R⁶ is independently C₁-C₆ alkyl, halo, hydroxy, —O(C₁-C₆ alkyl), —CN, C₁-C₆ alkyl—CN, C₁-C₆ alkyl-OH, or C₁-C₆ haloalkyl;
or two R⁶ groups attached to the same carbon atom are taken together with the carbon atom to which they are attached to form a spiro C₃-C₆ cycloalkyl or spiro 4- to 6-membered heterocyclyl;
X is hydrogen, C₁-C₆ alkyl, C₁-C₆ haloalkyl, C₁-C₆ alkyl-OH, C₁-C₆ alkyl—CN, C₃-C₆ cycloalkyl optionally substituted by one to five R⁸ groups, or 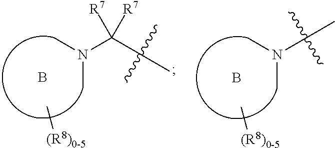
is a 4- to 7-membered heterocyclyl or 5- to 8- membered heteroaryl, wherein each heterocyclyl or heteroaryl optionally contains one to two additional heteroatoms selected from the group consisting of nitrogen, oxygen, and sulfur, and wherein each heterocyclyl or heteroaryl is optionally substituted by one to five R⁸ groups;
each R⁷ is independently hydrogen, C₁-C₆ alkyl, C₁-C₆ alkyl-OH, or C₁-C₆ haloalkyl;
or two R⁷ groups are taken together with the carbon atom to which they are attached to form a C3-cycloalkyl or 3- to 5- membered heterocyclyl; and
each R⁸ is independently halo, C₁-C₆ alkyl, C₁-C₆ alkyl—CN, C₁-C₆ alkyl-OH, C₁-C₆ haloalkyl, —CN, oxo, or —O(C₁-C₆ alkyl);
or two R⁸ groups are taken together with the carbon atom or atoms to which they are attached to form a spiro or fused C₃-C₅ cycloalkyl or 3- to 5-membered heterocyclyl.

20. The method of any of the previous claims, wherein the Cbl inhibitor compound is selected from the compounds in Table 1 or Table 2, or a stereoisomer or tautomer thereof, or a pharmaceutically acceptable salt thereof.

21. The method of any of the previous claims, wherein the Cbl inhibitor compound is according to Formula (II), or a pharmaceutically acceptable stereoisomer, tautomer, salt, or solvate thereof

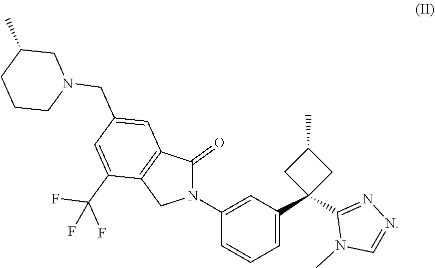 [SMILES: C[C@H]1CCCN(Cc2cc3c(c(C(F)(F)F)c2)CN(c2cccc([C@]4(c5nncn5C)C[C@@H](C)C4)c2)C3=O)C1]

22. The method of any of the previous claims, wherein the disease or condition is a cancer.

23. The method of any of the previous claims, wherein the disease or condition is a solid tumor.

24. The method of any of the previous claims, wherein the disease or condition is a hematological cancer.

25. The method of any of the previous claims, where the dose of the Cbl inhibitor compound is periodically increased.

26. The method of claim 25, wherein the dose of the Cbl inhibitor compound is increased from 15 mg to 25 mg over several daily doses.

27. The method of claim 25, wherein the dose of the Cbl inhibitor compound is increased from 25 mg to 50 mg over several daily doses.

28. The method of claim 25, wherein the dose of the Cbl inhibitor compound is increased from 15 mg to 25 mg to 50 mg over several daily doses.

29. The method of claim 25, wherein the dose of the Cbl inhibitor compound is increased from 15 mg daily for one week to 20 mg daily for one week to 25 mg daily for 3 weeks.

30. The method of claim 29, wherein the dose is maintained at 25 mg daily after the 25 mg daily for 3 weeks dose.

31. The method of claim 29, wherein the dose of the Cbl inhibitor compound is increased from the 25 mg daily for 3 weeks to 30 mg daily for 3 weeks to 25 mg thereafter.

32. The method of claim 25, wherein the dose of the Cbl inhibitor compound is increased from 15 mg daily for one week to 25 mg daily for one week to 30 mg daily.

33. The method of claim 25, wherein the dose of the Cbl inhibitor compound is increased from 15 mg daily to 35 mg daily over several weeks by increments of 5 mg each week.

34. The method of any one of claims 1-24, wherein a dosage of the Cbl inhibitor compound is selected from the group consisting of 15 mg once daily, 15 mg twice daily, 25 mg once daily, 25 mg twice daily, 50 mg once daily and 50 mg twice daily.

35. A method of treating or preventing a disease or condition in a subject in need thereof, comprising administering to the subject a Cbl inhibitor compound at a dose of 15 mg and subsequently administering the Cbl inhibitor compound at a dose of 25 mg.

36. A method of treating or preventing a disease or condition in a subject in need thereof, comprising administering to the subject a Cbl inhibitor compound at a dose of 15 mg, subsequently administering the Cbl inhibitor compound at a dose of 25 mg, and subsequently administering the Cbl inhibitor compound at a dose of 50 mg.

37. A method of treating or preventing a disease or condition in a subject in need thereof, comprising administering to the subject a Cbl inhibitor compound at a dose of 25 mg and subsequently administering the Cbl inhibitor compound at a dose of 50 mg.

38. A method of treating or preventing a disease or condition in a subject in need thereof, comprising administering to the subject a Cbl inhibitor compound at an increasing dose of mg daily for one week to 20 mg daily for one week to 25 mg daily for 3 weeks.

39. A method of treating or preventing a disease or condition in a subject in need thereof, comprising administering to the subject a Cbl inhibitor compound at an increasing dose of mg daily after the 25 mg daily for 3 weeks dose.

40. A method of treating or preventing a disease or condition in a subject in need thereof, comprising administering to the subject a Cbl inhibitor compound at an increasing dose of mg daily for 3 weeks to 30 mg daily for 3 weeks to 25 mg thereafter.

41. A method of treating or preventing a disease or condition in a subject in need thereof, comprising administering to the subject a Cbl inhibitor compound at an increasing dose of mg daily for one week to 25 mg daily for one week to 30 mg daily.

42. A method of treating or preventing a disease or condition in a subject in need thereof, comprising administering to the subject a Cbl inhibitor compound at an increasing dose of mg daily to 35 mg daily over several weeks by increments of 5 mg each week.

43. The method of any one of claims 35-42, wherein the Cbl inhibitor is a Cbl-b inhibitor.

44. The method of any one of claims 35-42, wherein the Cbl inhibitor is a c-Cbl inhibitor.

45. The method of any one of claims 35-43, wherein the Cbl inhibitor is a Cbl-b inhibitor and a c-Cbl inhibitor.

46. The method of any one of claims 35-45, wherein the Cbl inhibitor compound is according to Formula (I), or a pharmaceutically acceptable stereoisomer, tautomer, salt, or solvate thereof 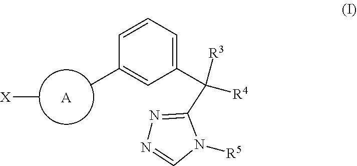
or a tautomer thereof, or a pharmaceutically acceptable salt thereof, wherein 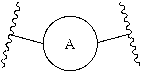
is, 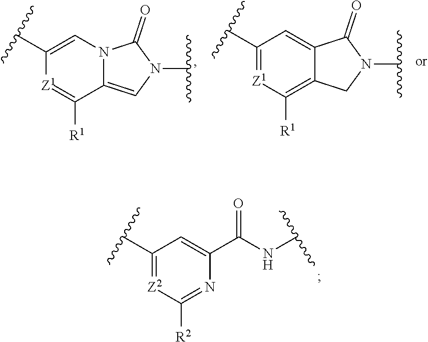
Z¹ is CH or nitrogen;
Z² is CH or nitrogen;
R¹ is —CF₃ or cyclopropyl;
R² is —CF₃ or cyclopropyl;
R³ is hydrogen, C₁-C₂ alkyl, or C₁-C₂ haloalkyl;
R⁴ is hydrogen, C₁-C₆ alkyl, C₁-C₆ haloalkyl, 4- to 8-membered heterocyclyl, or C₃-C₆ cycloalkyl, wherein the heterocyclyl or cycloalkyl groups are optionally substituted by one to five R⁶ groups;
or R³ and R⁴ are taken together with the carbon atom to which they are attached to form a C₃-C₅ cycloalkyl or 4- to 6-membered heterocyclyl, each of which is optionally substituted by one to five R⁶ groups;
R⁵ is hydrogen, C₁-C₆ alkyl, C₁-C₆ haloalkyl, or C₃-C₆ cycloalkyl;
each R⁶ is independently C₁-C₆ alkyl, halo, hydroxy, —O(C₁-C₆ alkyl), —CN, C₁-C₆ alkyl—CN, C₁-C₆ alkyl-OH, or C₁-C₆ haloalkyl;
or two R⁶ groups attached to the same carbon atom are taken together with the carbon atom to which they are attached to form a spiro C₃-C₆ cycloalkyl or spiro 4- to 6-membered heterocyclyl;
X is hydrogen, C₁-C₆ alkyl, C₁-C₆ haloalkyl, C₁-C₆ alkyl-OH, C₁-C₆ alkyl—CN, C₃-C₆ cycloalkyl optionally substituted by one to five R⁸ groups, or 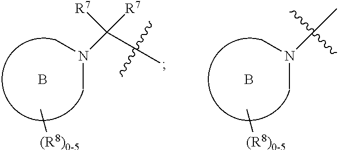
is a 4- to 7-membered heterocyclyl or 5- to 8- membered heteroaryl, wherein each heterocyclyl or heteroaryl optionally contains one to two additional heteroatoms selected from the group consisting of nitrogen, oxygen, and sulfur, and wherein each heterocyclyl or heteroaryl is optionally substituted by one to five R⁸ groups;
each R⁷ is independently hydrogen, C₁-C₆ alkyl, C₁-C₆ alkyl-OH, or C₁-C₆ haloalkyl;
or two R⁷ groups are taken together with the carbon atom to which they are attached to form a C3-cycloalkyl or 3- to 5- membered heterocyclyl; and
each R⁸ is independently halo, C₁-C₆ alkyl, C₁-C₆ alkyl—CN, C₁-C₆ alkyl-OH, C₁-C₆ haloalkyl, —CN, oxo, or —O(C₁-C₆ alkyl);
or two R⁸ groups are taken together with the carbon atom or atoms to which they are attached to form a spiro or fused C₃-C₅ cycloalkyl or 3- to 5-membered heterocyclyl.

47. The method of any one of claims 35-46, wherein the Cbl inhibitor compound is selected from the compounds in Table 1 or Table 2, or a stereoisomer or tautomer thereof, or a pharmaceutically acceptable salt thereof

48. The method of any one of claims 35-47, wherein the Cbl inhibitor compound is according to Formula (II), or a pharmaceutically acceptable stereoisomer, tautomer, salt, or solvate thereof

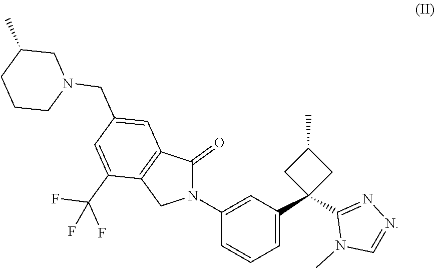 [SMILES: C[C@H]1CCCN(Cc2cc3c(c(C(F)(F)F)c2)CN(c2cccc([C@]4(c5nncn5C)C[C@@H](C)C4)c2)C3=O)C1]

49. The method of any one of claims 35-48, wherein the disease or condition is a cancer.

50. The method of any one of claims 35-49, wherein the disease or condition is a solid tumor.

51. The method of any one of claims 35-50, wherein the disease or condition is a hematological cancer.

52. The method of any one of claims 1-34, wherein the serotonin receptor antagonist and the Cbl inhibitor compound are in a single pharmaceutical composition.

53. A pharmaceutical composition comprising a Cbl inhibitor compound and a serotonin receptor antagonist.

54. The pharmaceutical composition of claim 53, wherein the Cbl inhibitor is a Cbl-b inhibitor.

55. The pharmaceutical composition of claim 53, wherein the Cbl inhibitor is a c-Cbl inhibitor.

56. The pharmaceutical composition of claim 53, wherein the Cbl inhibitor is a Cbl-b inhibitor and a c-Cbl inhibitor

57. The pharmaceutical composition of claim 53, wherein the Cbl inhibitor compound is according to Formula (I), or a pharmaceutically acceptable stereoisomer, tautomer, salt, or solvate thereof 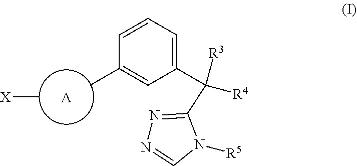
or a tautomer thereof, or a pharmaceutically acceptable salt thereof, wherein 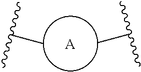
is, 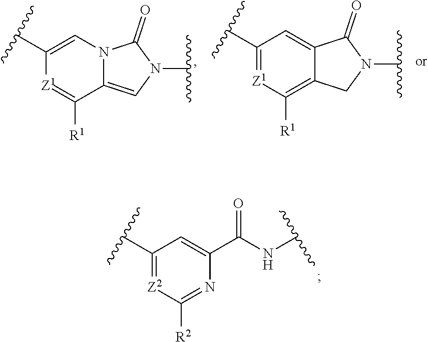
Z¹ is CH or nitrogen;
Z² is CH or nitrogen;
R¹ is —CF₃ or cyclopropyl;
R² is —CF₃ or cyclopropyl;
R³ is hydrogen, C₁-C₂ alkyl, or C₁-C₂ haloalkyl;
R⁴ is hydrogen, C₁-C₆ alkyl, C₁-C₆ haloalkyl, 4- to 8-membered heterocyclyl, or C₃-C₆ cycloalkyl, wherein the heterocyclyl or cycloalkyl groups are optionally substituted by one to five R⁶ groups;
or R³ and R⁴ are taken together with the carbon atom to which they are attached to form a C₃-C₅ cycloalkyl or 4- to 6-membered heterocyclyl, each of which is optionally substituted by one to five R⁶ groups;
R⁵ is hydrogen, C₁-C₆ alkyl, C₁-C₆ haloalkyl, or C₃-C₆ cycloalkyl;
each R⁶ is independently C₁-C₆ alkyl, halo, hydroxy, —O(C₁-C₆ alkyl), —CN, C₁-C₆ alkyl—CN, C₁-C₆ alkyl-OH, or C₁-C₆ haloalkyl;
or two R⁶ groups attached to the same carbon atom are taken together with the carbon atom to which they are attached to form a spiro C₃-C₆ cycloalkyl or spiro 4- to 6-membered heterocyclyl;
X is hydrogen, C₁-C₆ alkyl, C₁-C₆ haloalkyl, C₁-C₆ alkyl-OH, C₁-C₆ alkyl—CN, C₃-C₆ cycloalkyl optionally substituted by one to five R⁸ groups, or 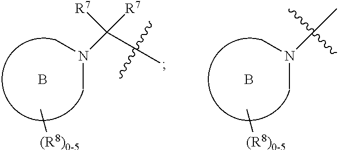
is a 4- to 7-membered heterocyclyl or 5- to 8- membered heteroaryl, wherein each heterocyclyl or heteroaryl optionally contains one to two additional heteroatoms selected from the group consisting of nitrogen, oxygen, and sulfur, and wherein each heterocyclyl or heteroaryl is optionally substituted by one to five R⁸ groups;
each R⁷ is independently hydrogen, C₁-C₆ alkyl, C₁-C₆ alkyl-OH, or C₁-C₆ haloalkyl;
or two R⁷ groups are taken together with the carbon atom to which they are attached to form a C3-cycloalkyl or 3- to 5- membered heterocyclyl; and
each R⁸ is independently halo, C₁-C₆ alkyl, C₁-C₆ alkyl—CN, C₁-C₆ alkyl-OH, C₁-C₆ haloalkyl, —CN, oxo, or —O(C₁-C₆ alkyl);
or two R⁸ groups are taken together with the carbon atom or atoms to which they are attached to form a spiro or fused C₃-C₅ cycloalkyl or 3- to 5-membered heterocyclyl.

58. The pharmaceutical composition of any one of claims 53-57, wherein the Cbl inhibitor compound is selected from the compounds in Table 1 or Table 2, or a stereoisomer or tautomer thereof, or a pharmaceutically acceptable salt thereof

59. The pharmaceutical composition of any one of claims 53-58, wherein the Cbl inhibitor compound is according to Formula (II), or a pharmaceutically acceptable stereoisomer, tautomer, salt, or solvate thereof

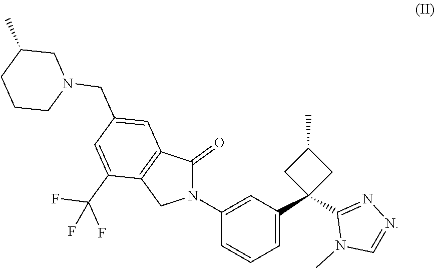 [SMILES: C[C@H]1CCCN(Cc2cc3c(c(C(F)(F)F)c2)CN(c2cccc([C@]4(c5nncn5C)C[C@@H](C)C4)c2)C3=O)C1]

60. The pharmaceutical composition of any one of claims 53-59, wherein the serotonin receptor antagonist is selected from the group consisting of ondansetron, granisetron, palonosetron, dolasetron, and combinations thereof
